(19) United States
(12) Patent Application Publication
(10) Pub. No.: US 2015/0354014 A1
(43) Pub. Date: Dec. 10, 2015

(54) Compositions, methods and kits for diagnosis of carriers of mutations in the BRCA1 and BRCA2 genes and early diagnosis of cancerous disorders associated with mutations in BRCA1 and BRCA 2 genes

(71) Applicant: Hadasit Medical Research Services and Development Ltd., Jerusalem (IL)

(72) Inventors: Asher SALMON, Jerusalem (IL); Tamar PERETZ, Jerusalem (IL)

(21) Appl. No.: 14/834,989

(22) Filed: Aug. 25, 2015

Related U.S. Application Data
(63) Continuation of application No. 12/668,154, filed on Apr. 8, 2010.
Application No. 12/668,154 is a continuation-in-part of application No. PCT/IL2008/000934, filed on Jul. 8, 2008.

(30) Foreign Application Priority Data
Jul. 8, 2007 (IL) 184478

(51) Int. Cl. C12Q 1/68

(52) U.S. Cl. CPC C12Q 1/6886; C12Q 2600/156; C12Q 2600/158

(57) ABSTRACT

The present invention relates to diagnostic compositions methods and kits for the detection of carriers of mutations in the BRCA1 and BRCA2 genes. The detection is based on the use of detecting nucleic acids or amino acid based molecules, specific for determination of the expression of at least six marker genes of the invention, in a test sample. The invention thereby provides methods compositions and kits for the diagnosis of cancerous disorders associated with mutations in the BRCA1 and BRCA2 genes, specifically, of ovarian and breast cancer.

### FIELD OF THE INVENTION

The invention relates to early diagnosis of cancerous disorders. More particularly, the invention relates to compositions methods and kits based on measuring differential expression of specific marker genes, for the diagnosis of carriers of mutations in the BRCA1 and BRCA2 genes and thereby, the diagnosis of cancerous disorders associated therewith, specifically, of ovarian and breast cancer.

### BACKGROUND OF THE INVENTION

All publications mentioned throughout this application are fully incorporated herein by reference, including all references cited therein.

Diagnostic markers are important for early diagnosis of many diseases, as well as predicting response to treatment, monitoring treatment and determining prognosis of such diseases.

Mutations in the breast and ovarian cancer susceptibility genes BRCA1 and BRCA2 are found in a high proportion of multiple-case families with breast and ovarian cancer [Antoniou, A. C. et al. Genetic Epidemiology 25:190-202 (2003)]. Carriers of mutations in BRCA1 or BRCA2 genes have up to 80% lifetime risk of developing breast and ovarian cancers and elevated risk of developing other types of cancer, such as prostate and pancreas. Mutations in the BRCA1 gene account for 50% of familial breast cancer cases. Mutations in BRCA2 account for 30% of familial breast cancer cases and are also linked to male breast cancer.

About 80% of all alterations in BRCA1 and BRCA2 tumors are frame shift or nonsense mutations, and yield a truncated protein product [Breast cancer Information Core—BIC at http://www.nhgri.nih.gov/Intramural_research/Lab_transfer/Bic]. The types of mutation differ in distribution depending on ethnicity and geographic location. There is increasing evidence that hereditary cancer syndromes resulting from germ line mutations in cancer susceptibility genes lead to organ-specific cancers with distinct histological phenotypes. The hereditary breast tumors that result from germ line BRCA1 and BRCA2 mutations exemplify this phenomenon. In recent years, it has been demonstrated that BRCA1 and BRCA2 breast carcinomas differs from sporadic breast cancer of age-matched controls and from non-BR CA1/2 familial breast carcinomas in their morphological, immunophenotypic and molecular characteristics [Phillips K. A. Journal of Clinical Oncology 18:107s-112s (2000)].

The structurally distinct proteins encoded by BRCA1 and BRCA2 regulate numerous cellular functions, including DNA repair, chromosomal segregation, gene transcription, cell-cycle arrest and apoptosis. BRCA1 and BRCA2 are considered to be “gatekeepers”: genes which, when mutated or abnormally expressed, cause disruption of normal cell biology, interrupt cell division or death control, and promote the outgrowth of cancer cells. Recent reports have provided insight into the role of BRCA1 and BRCA2 in the cellular response to DNA damage [Tutt A. et al. The EMBO Journal 20:4704-4716 (2001)]. Several groups have demonstrated that BRCA1- or BRCA2-deficient rodent cells or human tumors are specifically deficient in DNA repair via homologous recombination, whereas, when measured, non-homologous recombination remains intact after double-strand DNA breaks. Moreover, the correlation between BRCA1 or BRCA2 mutation and alterations in p53, HER 2 and Myc gene expression as well as alterations in cell-cycle regulation have been shown in breast carcinoma patients [Venkitaraman A R. Journal of Cell Science. 114:3591-8 (2001)]. Together, these data imply that accumulation of somatic genetic changes during tumor progression may follow a unique pathway in individuals genetically predisposed to cancer.

As mentioned above, BRCA1 and BRCA2 proteins maintain genomic stability through an involvement in DNA repair processes. Mutations in BRCA1 and BRCA2 seem to predispose cells to an increased risk of mutagenesis and transformation after exposure to radiation. It was shown recently that normal human fibroblasts and lymphoblastoid cells with heterozygous BRCA1 and BRCA2 mutations seem to have increased radio sensitivity [Buchholz, T. A. et al. International Journal of Cancer 97:557-561 (2002)]. Previous study of the present inventors on short-term lymphocyte cultures, provided additional evidence that heterozygous mutation carriers have a different response to DNA damage compared with non-carriers [Kote-Jarai, Z. et al. British Journal of Cancer 94:308-310 (2006)]. The characterization of BRCA1/2 RNA expression profile of human fibroblasts from healthy mutation carriers has been described using spotted cDNA microarray [Kote-Jarai, Z. et al. Clinical Cancer Research 12:3896-901 (2006)]. This study shows a significant difference in gene expression profiling in heterozygous BRCA1 and BRCA2 mutation carriers as compared to non-carriers following induced DNA damage caused by exposure to irradiation.

The present invention discloses marker genes differentially expressed in lymphocytes from BRCA1 and BRCA2 carriers versus non-carriers following irradiation stress. These marker genes are used by the compositions, kits and methods of the invention as a tool for detecting carriers and thereby for early detection of proliferative disorders and particularly, of breast and ovarian carcinomas.

It is therefore one object of the invention to provide a simple diagnostic composition comprising at least one detecting molecule specific for quantitative determination of the expression profile of a collection of marker genes. Another object of the invention is to provide a set of pre-determined marker genes expression level cutoff values useful for comparison with the corresponding expression levels in a tested subject for the diagnosis of BRCA1 or BRCA2 genes mutation carriers.

Yet another object of the invention is to provide a simple, inexpensive, and clear test to distinguish between BRCA1 or BRCA2 genes mutation carriers and non-carriers.

As indicated above, carriers of mutations in BRCA1 or BRCA2 genes exhibit increased predisposition to cancerous disorders Therefore, another object of the invention is to provide diagnostic method for early detection of cancerous disorders associated with mutations in these genes, particularly of breast and ovarian cancer. This method is based on quantitative determination of the expression of at least one marker gene described by the invention.

A further object of the invention is to provide diagnostic kit for detection of carriers of BRCA1 and BRCA2 gene mutations and thereby the diagnosis of cancerous disorders associated with mutations in BRCA1 or BRCA2 genes.

These and other objects of the invention will become apparent as the description proceeds.

### SUMMARY OF THE INVENTION

In a first aspect, the invention relates to a composition comprising detecting molecules specific for determination of the expression of at least six marker genes, wherein said detecting molecules are selected from isolated detecting nucleic acid molecules and isolated detecting amino acid molecules. It should be noted that at least six marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6orf111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12, as set forth in Table 4. According to this embodiment, the composition of the invention is used for determining the level of expression of at least six of said marker genes in a biological test sample of a mammalian subject.

According to another embodiment, the composition of the invention comprises detecting molecules specific for at least six marker genes selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2, as set forth in Table 8. It should be appreciated that the composition of the invention is specifically used for determining the level of expression of at least six of the marker genes indicated by the invention in a biological test sample of a mammalian subject.

According to one specific embodiment, the composition of the invention is specifically applicable for the detection of at least one mutation in at least one of BRCA1 and BRCA2 genes in a biological sample of a mammalian subject.

In another aspect, the invention contemplates a method for the detection of at least one mutation in at least one of BRCA1 and BRCA2 genes in a biological test sample of a mammalian subject. According to a specific embodiment, the method of the invention comprises the steps of:

(a) determining the level of expression of at least six marker genes in said test sample and optionally in a suitable control sample, wherein said at least six marker genes are selected from any one of:
(i) a group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2;
(ii) the group as defined in (i) further consisting of: RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6orf111), splicing factor, arginine/serine-rich 18;
(iii) the group as defined in (i) further consisting of: RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6orf111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12, as set forth in Table 4;
(b) determining the level of expression of at least one control gene in said test sample and optionally, in a suitable control sample;
(c) comparing the expression values obtained in steps (a) and (b) of each marker gene in said test sample with a corresponding predetermined cutoff value of each of said marker genes;
(d) determining whether said expression value of each said marker gene is positive and thereby belongs to a pre-established carrier population or is negative and belongs to a pre-established non-carrier population;

It should be appreciated that the presence of at least six marker genes with a positive expression value indicates that said subject is a carrier of at least one mutation of at least one of BRCA1 or BRCA2 gene.

Another aspect of the invention relates to a kit comprising:

(a) means for obtaining a sample of a mammalian subject;
(b) detecting molecules specific for determining the level of expression of at least six marker genes, wherein said detecting molecules are selected from isolated detecting nucleic acid molecules and isolated detecting amino acid molecules, and wherein said at least six marker genes are selected from any one of:
(i) a group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2;
(ii) the group as defined in (i) further consisting of: RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6orf111), splicing factor, arginine/serine-rich 18;
(iii) the group as defined in (i) further consisting of: RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6orf111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12, as set forth in Table 4;
(c) at least one detecting molecule specific for determining the expression of at least one control gene;
(d) optionally, at least one control sample selected from a negative control sample and a positive control sample;
(e) instructions for carrying out the detection and quantification of expression of said at least six marker genes and of at least one control gene in said sample, and for obtaining expression values of each of said marker genes; and
(f) instructions for comparing the expression values of each marker gene in said test sample with a corresponding predetermined cutoff value of each of said marker genes and determining a positive or negative results thereby evaluating the differential expression of said marker gene in said sample.

These and other aspects of the invention will become apparent by the hand of the following figures and examples.

### BRIEF DESCRIPTION OF THE FIGURES

FIG. 1A-1C. Heat map of gene expression profile of lymphocytes from BRCA1 mutation carriers and control non-carriers (A) or BRCA2 carriers and control non-carriers (B). Data analysis by Expression Console Software (Affymetrix) represented in Figure (C) Only the genes expressed in significantly distinct manner (with p-value <0.05) were selected for analysis. Abbreviations: cont. (control).

FIG. 2. Principal components analysis (PCA) of gene profile in BRCA1 and BRCA2 mutation carriers and control. Abbreviations: gr. (group), C (control), Ma (mapping).

FIG. 3A-3C. ANOVA analysis of BRCA1 (yellow), BRCA2 (blue) and control (red) gene expression.

FIG. 3A. Clustering of the whole gene set. Note the homogenous clustering of BRCA2 as compared to the somewhat more heterogeneous clustering of BRCA1. FIG. 3B. An enlargement of a sample cluster. FIG. 3C. Cluster of 11 genes that were significantly under-expressed in BRCA1 in comparison to BRCA2 and control.

FIG. 4A-4B. Graphic presentation of functional groups of all genes having differentionl expression in samples of BRCA1 mutation carriers. FIG. 4A demonstrate genes which are up regulated as compared to a non-carrier control and FIG. 4B demonstrate genes which are down regulated in BRCA1 mutation sample. Abbreviations: bin. (binding), sig. (signal), trans. (transducer), ac. (activity), tm. (transmembrane), Rec. (receptor), Ag. (antigen), reg. (regulator), Ha. (heavy), met. (metal), pr. (protein), Unf. (unfolded), Enz (enzymatic).

FIG. 5A-5B. Graphic presentation of functional groups of all genes having differentionl expression in samples of BRCA2 mutation carriers. FIG. 5A demonstrate genes which are up regulated as compared to a non-carrier control and FIG. 5B demonstrate genes which are down regulated in BRCA2 mutation sample. Abbreviations: bin. (binding), ac. (activity), cat. (catalytic), nuc. (nucleotide), pr. (protein), Enz (enzymatic), kin. (kinase), sin. (single), str. (strand), lip. (lipid), cons. (constituent), stru. (structured).

FIG. 6. Gene Ontology analysis of the genes differentially expressed, with most similar gene expression consistent into each group.

### DETAILED DESCRIPTION OF THE INVENTION

The present invention discloses characterization of the gene expression profile in freshly cultured lymphocytes obtained from non-carrier women as compared to carriers of mutations in either BRCA1 or BRCA2.

BRCA1 and BRCA2 up-regulate tumor suppressor and growth-inhibitory genes and repress cell proliferation genes, serving as transcriptional co-activators depending on the specific target gene. Despite a large number of studies on BRCA1 and BRCA2 genes, the exact role of BRCA1 and BRCA2 regulators of DNA repair, transcription, and the cell cycle in response to DNA damage is still unclear, and mechanisms underlying the tissue specificity of their tumor-suppressive property remain speculative.

As shown by the following Examples, the inventors assessed gene expression variation between irradiated and non-irradiated lymphocytes isolated from non-carrier subjects and carriers of mutations in BRCA1, BRCA2 or both. This comparison revealed significant differences in gene expression profile of a particular group of twenty, and more specifically eighteen marker genes, between groups of carriers of mutations in any one of BRCA1 and BRCA2 genes and the control non-carrier groups.

A further study of the gene expression differences between normal non-carrier subjects and carriers of BRCA1 and BRCA2 mutations revealed specific expression values for the marker gene group, a deviation from which of at least six such genes is indicative of an increased likelihood for the presence of at least one mutation in any one of BRCA1 and/or BRCA2 in a tested subject. This discovery is beneficial, for example, as a cost-effective screening method for detection of cancer-predisposed subjects for follow up and possible prophylaxis as well as suitable treatment upon detection of relevant tumors.

Thus, according to a first aspect, the invention relates to a composition comprising at least one detecting molecule or a collection of at least two detecting molecules specific for determination of the expression of at least one marker gene or a collection of at least two marker genes. More specifically, these marker genes may be selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6orf111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, and are as set forth in Table 4, or any collection or combination thereof. It should be noted that the composition of the invention may be specifically applicable for determining the level of expression (also referred to herein as “profiling” or “expression pattern”) of at least one of said marker genes in a biological test sample of a mammalian subject. According to certain embodiments, the composition of the invention may be specifically applicable for determining the level of expression of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of said marker genes in a biological test sample of a mammalian subject.

In certain embodiments, the present invention provides a composition comprising detecting molecules specific for determination of the expression of at least six marker genes. The detecting molecules of the invention may be any one of isolated detecting nucleic acid molecules and isolated detecting amino acid molecules, or any combinations thereof. These at least six marker genes may be selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6orf111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12, as set forth in Table 4. The purpose of this composition is the determination of the level of expression of at least six of the marker genes in a biological test sample of a mammalian subject.

According to another embodiment of this composition, at least six marker genes may be selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6orf111), splicing factor, arginine/serine-rich 18, as set forth in Table 7.

In yet another embodiment, the marker genes may be selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2, as set fourth in Table 8.

In a particular embodiment, the invention further provides a composition comprising detecting molecules specific for: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2. According to certain embodiments, said composition may further comprises detecting molecules specific for at least one of RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6orf111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12. It should be noted that any of the methods and kits of the invention described herein after may use such particular composition as indicated herein.

According to one embodiment, the detecting molecules are specific for quantitative or qualitative determination of expression of said marker genes. Preferably, the detecting molecules used by the invention may be specifically suitable for quantitative determination of expression of any of the marker genes used by the composition of the invention, as set forth in any one of Table 4, Table 7 and Table 8.

According to one embodiment, the detecting molecule used by the composition of the invention may be an isolated nucleic acid molecule or an isolated amino acid molecule. It should be appreciated that the composition of the invention may comprise both, nucleic acid based detecting molecules and amino acid based detecting molecules. Thus, the invention further contemplates the use of a combination of proteins or polypeptides in combination with polynucleotides so as to measure one or more products of one or more of the marker genes of the invention, in any combination thereof.

As used herein, “nucleic acid(s)” is interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 8 to about 1,000 nucleotides long. Although oligonucleotides of 5 to 100 nucleotides are useful in the invention, preferred oligonucleotides range from about 5 to about 15 bases in length, from about 5 to about 20 bases in length, from about 5 to about 25 bases in length, from about 5 to about 30 bases in length, from about 5 to about 40 bases in length or from about 5 to about 50 bases in length. More specifically, the detecting oligonucleotides molecule used by the composition of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 bases in length.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

As indicated above, the detecting molecules of the invention may be amino acid based molecules that may be referred to as protein/s or polypeptide/s. As used herein, the terms “protein” and “polypeptide” are used interchangeably to refer to a chain of amino acids linked together by peptide bonds. In a specific embodiment, a protein is composed of less than 200, less than 175, less than 150, less than 125, less than 100, less than 50, less than 45, less than 40, less than 35, less than 30, less than 25, less than 20, less than 15, less than 10, or less than 5 amino acids linked together by peptide bonds.

In another embodiment, a protein is composed of at least 200, at least 250, at least 300, at least 350, at least 400, at least 450, at least 500 or more amino acids linked together by peptide bonds.

According to one specific embodiment, the isolated detecting nucleic acid molecules comprised within the composition of the invention may be isolated oligonucleotides. Each oligonucleotide specifically or/and selectively hybridizes to a nucleic acid sequence of the RNA products of at least one marker gene selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6orf111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4.

In some embodiments, where the composition's detecting nucleic acid molecules are isolated oligonucleotides, each oligonucleotide specifically hybridizing to a nucleic acid sequence of the RNA products of at least one of the at least six marker genes. According to certain embodiments, these at least six marker genes may be selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6orf111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12, as indicated in the twenty gene list as set forth in Table 4. The aforementioned detecting oligonucleotide molecules are used for determining the level of expression of at least six marker genes in the test sample, and a differential expression of at least six such genes in the test sample as compared to a control population is indicative of at least one mutation in at least one of BRCA1 and BRCA2 genes in the subject, and thereby of an increased genetic predisposition of the subject to a cancerous disorder associated with mutations in any one of BRCA1 and BRCA2 genes.

In another embodiment, the composition of the invention may comprise oligonucleotides that specifically hybridize to nucleic acid sequences of RNA products of at least one of at least six marker genes selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6orf111), splicing factor, arginine/serine-rich 18, as indicated in the eighteen genes list as set forth in Table 7. The detecting oligonucleotide molecules are used for determining the level of expression of the at least six marker gene in a sample, and a differential expression of at least six such genes in the test sample as compared to a control population is indicative of at least one mutation in at least one of BRCA1 and BRCA2 genes in the subject, and thereby of an increased genetic predisposition of the subject to a cancerous disorder associated with mutations in any one of BRCA1 and BRCA2 genes.

Still another embodiment relates to the composition of the invention which comprises isolated detecting oligonucleotides, each oligonucleotide specifically hybridizes to a nucleic acid sequences of RNA products of at least one of at least six marker genes selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2, as shown in the thirteen genes list as set forth in Table 8. The detecting oligonucleotide molecules are used for determining the level of expression of the at least six marker gene in a sample, and a differential expression of at least six of the marker genes in the test sample as compared to a control population is indicative of at least one mutation in at least one of BRCA1 and BRCA2 genes in the subject, and thereby of an increased genetic predisposition of the subject to a cancerous disorder associated with mutations in any one of BRCA1 and BRCA2 genes.

As indicated above, the compositions of the invention comprise oligonucleotides that specifically hybridize to nucleic acid sequences of RNA products of the marker gene. As used herein, the term “hybridize” refers to a process where two complementary nucleic acid strands anneal to each other under appropriately stringent conditions. Hybridizations are typically and preferably conducted with probe-length nucleic acid molecules, preferably 5-200 nucleotides in length, 5-100, 5-50, 5-40, 5-30 or 5-20.

As used herein “selective or specific hybridization” in the context of this invention refers to a hybridization which occurs between a polynucleotide encompassed by the invention and an RNA product of any of the marker gene of the invention, wherein the hybridization is such that the polynucleotide binds to the RNA products of the marker gene of the invention preferentially to any RNA products of other gene products in the tested sample. In a preferred embodiment a polynucleotide which “selectively hybridizes” is one which hybridizes with a selectivity of greater than 60%, greater than 70%, greater than 80%, greater than 90% and most preferably on 100% (i.e. cross hybridization with other RNA species preferably occurs at less than 40%, less than 30%, less than 20%, less than 10%). As would be understood to a person skilled in the art, a detecting polynucleotide which “selectively hybridizes” to the RNA product of a marker gene of the invention can be designed taking into account the length and composition.

As used herein, “specifically hybridizes”, “specific hybridization” refers to hybridization which occurs when two nucleic acid sequences are substantially complementary (at least about 60% complementary over a stretch of at least 5 to 25 nucleotides, preferably at least about 70%, 75%, 80% or 85% complementary, more preferably at least about 90% complementary, and most preferably, about 95% complementary).

The measuring of the expression of the RNA product of any one of the marker genes and combination of marker genes of the invention can be done by using those polynucleotides as detecting molecules, which are specific and/or selective for the RNA products of the marker genes of the invention to quantitate the expression of the RNA product. In a specific embodiment of the invention, the polynucleotides which are specific and/or selective for the RNA products may be probes or primers. It should be further appreciated that the composition of the invention may comprise, as an oligonucleotide-based detection molecule, both primers and probes.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the composition of the invention may comprise 10, 11, 12, 13, 14, 15, 16, 17, 18, 19, 20, 21, 22, 23, 24, 25, 26, 27, 28, 29 or 30 nucleotides. The factors involved in determining the appropriate length of primer are readily known to one of ordinary skill in the art.

As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe is at least 5 or preferably, 8 nucleotides in length and less than the length of a complete gene. A probe may be 5, 6, 7, 8, 9, 10, 11, 12, 13, 14, 15, 16, 17, 18, 19, 20, 21, 22, 23, 24, 25, 26, 27, 28, 29, 30, 40, 50, 75, 100, 150, 200, 250, 400, 500 and up to 2000 nucleotides in length as long as it is less than the full length of the target gene. Probes can include oligonucleotides modified so as to have a tag which is detectable by fluorescence, chemiluminescence and the like. The probe can also be modified so as to have both a detectable tag and a quencher molecule, for example TaqMan® and Molecular Beacon® probes, that will be described in detail below.

The oligonucleotides and analogs thereof may be RNA or DNA, or analogs of RNA or DNA, commonly referred to as antisense oligomers or antisense oligonucleotides. Such RNA or DNA analogs comprise, but are not limited to, 2-′O-alkyl sugar modifications, methylphosphonate, phosphorothiate, phosphorodithioate, formacetal, 3-thioformacetal, sulfone, sulfamate, and nitroxide backbone modifications, and analogs wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs.

Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

According to another preferred embodiment, when the detecting molecule is an oligonucleotide, the expression level of any of the marker genes may be determined using at least one nucleic acid amplification assay, such as a Real-Time PCR, micro arrays, PCR, in situ Hybridization or Comparative Genomic Hybridization.

In further embodiments, the oligonucleotides are any one of a pair of primer or nucleotide probe. Thus, it should be appreciated that also the level of expression of at least six of the marker genes is determined using a nucleic acid amplification assay selected from the group consisting of: a Real-Time PCR, micro arrays, PCR, in situ Hybridization and Comparative Genomic Hybridization.

The term “amplify”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction. “Polymerase chain reaction” or “PCR” refers to an in vitro method for amplifying a specific nucleic acid template sequence. The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers.

Real time nucleic acid amplification and detection methods are efficient for sequence identification and quantification of a target since no pre-hybridization amplification is required. Amplification and hybridization are combined in a single step and can be performed in a fully automated, large-scale, closed-tube format.

Methods that use hybridization-triggered fluorescent probes for real time PCR are based either on a quench-release fluorescence of a probe digested by DNA Polymerase (e.g., methods using TaqMan®, MGB-TaqMan®), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan® and MGB-TaqMan® the 5′-exonuclease activity of the approaching DNA Polymerase cleaves a probe between fluorophore and quencher thus releasing fluorescence.

Thus, a “real time PCR” assay provides dynamic fluorescence detection of amplified marker gene products produced in a PCR amplification reaction. During PCR, the amplified products created using suitable primers hybridize to probe nucleic acids (TaqMan® probe, for example), which may be labeled according to some embodiments with both a reporter dye and a quencher dye. When these two dyes are in close proximity, i.e. both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR.

The method and hybridization assays using self-quenching fluorescence probes with and/or without internal controls for detection of nucleic acid application products are known in the art, for example, U.S. Pat. Nos. 6,258,569; 6,030,787; 5,952,202; 5,876,930; 5,866,336; 5,736,333; 5,723,591; 5,691,146; and 5,538,848.

More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan® (Perkin Elmer, Foster City, Calif.), is performed with a transcript-specific antisense probe. This probe is specific for the PCR product (e.g. a nucleic acid fragment derived from a gene) and is prepared with a quencher and fluorescent reporter probe attached to the 5′ end of the oligonucleotide. Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions can be performed in any solid support, for example, slides, microplates, 96 well plates, 384 well plates and the like so that samples derived from many individuals are processed and measured simultaneously. The TaqMan® system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces fluorescence proportional to the amount of PCR product.

Both TaqMan® and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other known systems to quantitatively measure mRNA expression products include Molecular Beacons® which uses a probe having a fluorescent molecule and a quencher molecule, the probe capable of forming a hairpin structure such that when in the hairpin form, the fluorescence molecule is quenched, and when hybridized the fluorescence increases giving a quantitative measurement of gene expression.

In one embodiment, the polynucleotide-based detection molecules of the invention may be in the form of nucleic acid probes which can be spotted onto an array to measure RNA from the sample of a subject to be diagnosed.

As defined herein, a “nucleic acid array” refers to a plurality of nucleic acids (or “nucleic acid members”), optionally attached to a support where each of the nucleic acid members is attached to a support in a unique pre-selected and defined region. These nucleic acid sequences are used herein as detecting nucleic acid molecules. In one embodiment, the nucleic acid member attached to the surface of the support is DNA. In a preferred embodiment, the nucleic acid member attached to the surface of the support is either cDNA or oligonucleotides. In another embodiment, the nucleic acid member attached to the surface of the support is cDNA synthesized by polymerase chain reaction (PCR). In another embodiment, a “nucleic acid array” refers to a plurality of unique nucleic acid detecting molecules attached to nitrocellulose or other membranes used in Southern and/or Northern blotting techniques.

For oligonucleotide-based arrays, the selection of oligonucleotides corresponding to the gene of interest which are useful as probes is well understood in the art.

More particularly, it is important to choose regions which will permit hybridization to the target nucleic acids. Factors such as the Tm of the oligonucleotide, the percent GC content, the degree of secondary structure and the length of nucleic acid are important factors.

According to this embodiment, the detecting molecule may be in the form of probe corresponding and thereby hybridizing to any region or part of the marker gene. For example, these probes may be a set of corresponding 5′ ends or a set of corresponding 3′ ends or a set of corresponding internal coding regions. Of course, mixtures of a 5′ end of one gene may be used as a target or a probe in combination with a 3′ end of another gene to achieve the same result of measuring the levels of expression of the marker gene.

As used herein, the “5′ end” refers to the end of an mRNA up to the first 1000 nucleotides or one third of the mRNA (where the full length of the mRNA does not include the poly A tail), starting at the first nucleotide of the mRNA. The “5′ region” of a gene refers to a polynucleotide (double-stranded or single-stranded) located within or at the 5′ end of a gene, and includes, but is not limited to, the 5′ untranslated region, if that is present, and the 5′ protein coding region of a gene. The 5′ region is not shorter than 8 nucleotides in length and not longer than 1000 nucleotides in length. Other possible lengths of the 5′ region include but are not limited to 10, 20, 25, 50, 100, 200, 400, and 500 nucleotides.

As used herein, the “3′ end” refers to the end of an mRNA up to the last 1000 nucleotides or one third of the mRNA, where the 3′ terminal nucleotide is that terminal nucleotide of the coding or untranslated region that adjoins the poly-A tail, if one is present. That is, the 3′ end of an mRNA does not include the poly-A tail, if one is present. The “3′ region” of a gene refers to a polynucleotide (double-stranded or single-stranded) located within or at the 3′ end of a gene, and includes, but is not limited to, the 3′ untranslated region, if that is present, and the 3′ protein coding region of a gene. The 3′ region is not shorter than 8 nucleotides in length and not longer than 1000 nucleotides in length. Other possible lengths of the 3′ region include but are not limited to 10, 20, 25, 50, 100, 200, 400, and 500 nucleotides. As used herein, the “internal coding region” of a gene refers to a polynucleotide (double-stranded or single-stranded) located between the 5′ region and the 3′ region of a gene as defined herein.

The “internal coding region” is not shorter than 8 nucleotides in length and not longer than 1000 nucleotides in length. Other possible lengths of the “internal coding region” include but are not limited to 10, 20, 25, 50, 100, 200, 400, and 500 nucleotides. The 5′, 3′ and internal regions are non-overlapping and may, but need not be, contiguous, and may, but need not, add up to the full length of the corresponding gene.

As indicated above, assay based on micro array or RT-PCR may involve attaching or spotting of the probes in a solid support. As used herein, the terms “attaching” and “spotting” refer to a process of depositing a nucleic acid onto a substrate to form a nucleic acid array such that the nucleic acid is stably bound to the substrate via covalent bonds, hydrogen bonds or ionic interactions.

As used herein, “stably associated” or “stably bound” refers to a nucleic acid that is stably bound to a solid substrate to form an array via covalent bonds, hydrogen bonds or ionic interactions such that the nucleic acid retains its unique pre-selected position relative to all other nucleic acids that are stably associated with an array, or to all other pre-selected regions on the solid substrate under conditions in which an array is typically analyzed (i.e., during one or more steps of hybridization, washes, and/or scanning, etc.).

As used herein, “substrate” or “support” or “solid support”, when referring to an array, refers to a material having a rigid or semi-rigid surface. The support may be biological, non-biological, organic, inorganic, or a combination of any of these, existing as particles, strands, precipitates, gels, sheets, tubing, spheres, beads, containers, capillaries, pads, slices, films, plates, slides, chips, etc. Often, the substrate is a silicon or glass surface, (poly)tetrafluoroethylene, (poly) vinylidendifluoride, polystyrene, polycarbonate, a charged membrane, such as nylon or nitrocellulose, or combinations thereof. Preferably, at least one surface of the substrate will be substantially flat. The support may optionally contain reactive groups, including, but not limited to, carboxyl, amino, hydroxyl, thiol, and the like. In one embodiment, the support may be optically transparent.

It should be noted that other nucleic acid based assays may be used for quantitative measurement of the marker genes expression level. For example, Nuclease protection assays (including both ribonuclease protection assays and S1 nuclease assays) can be used to detect and quantitate the RNA products of the marker genes of the invention. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments.

It should be further noted that a standard Northern blot assay can also be used to ascertain an RNA transcript size and the relative amounts of RNA products of the marker gene of the invention, in accordance with conventional Northern hybridization techniques known to those persons of ordinary skill in the art.

The invention further contemplates the use of amino acid based molecules such as proteins or polypeptides as detecting molecules disclosed herein and would be known by a person skilled in the art to measure the protein products of the marker genes of the invention. Techniques known to persons skilled in the art (for example, techniques such as Western Blotting, Immunoprecipitation, ELISAs, protein microarray analysis and the like) can then be used to measure the level of protein products corresponding to the marker genes of the invention. As would be understood to a person skilled in the art, the measure of the level of expression of the protein products of the marker genes of the invention requires a protein which specifically and/or selectively binds to one or more of the protein products corresponding to each marker genes of the invention.

Thus, according to a particular embodiment, the invention provides an alternative composition comprising as the detection molecules, isolated amino acid molecules. Accordingly, each of such detection molecules may be an isolated polypeptide which binds selectively and specifically to a protein product of at least one marker gene selected from the group consisting of RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6orf111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4.

In specific embodiments, the detecting amino acid molecules are isolated antibodies, with each antibody binding selectively to a protein product of at least one of the at least six marker genes. Using these antibodies, the level of expression of the at least six marker genes is determined using an immunoassay which is selected from the group consisting of an ELISA, a RIA, a slot blot, a dot blot, immunohistochemical assay, FACS, a radio-imaging assay and a Western blot.

According to certain embodiments, the specific antibodies may be used by the invention for determining the level of expression of at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4, in a biological test sample of a mammalian subject.

In yet other embodiments, the specific antibodies may be used by the invention for determining the level of expression of at least six, at least seven, at least eight, at least nine, at least ten, at least eleven, at least twelve, at least thirteen, at least fourteen, at least fifteen, at least sixteen, at least seventeen, or at least eighteen of the eighteen marker genes listed in Table 7, in a biological test sample of a mammalian subject.

In other embodiments, the specific antibodies may be used by the invention for determining the level of expression of at least six, at least seven, at least eight, at least nine, at least ten, at least eleven, at least twelve or at least thirteen, of the thirteen marker genes listed in Table 8, in a biological test sample of a mammalian subject.

As indicated above, the specific antibodies used by the invention selectively bind to the protein product of the marker genes. “selectively bind” in the context of proteins encompassed by the invention refers to the specific interaction of a any two of a peptide, a protein, a polypeptide an antibody, wherein the interaction preferentially occurs as between any two of a peptide, protein, polypeptide and antibody preferentially as compared with any other peptide, protein, polypeptide and antibody. For example, when the two molecules are protein molecules, a structure on the first molecule recognizes and binds to a structure on the second molecule, rather than to other proteins. “Selective binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

As indicated above, according to some embodiment, the detecting molecules of the composition of the invention may be an isolated and purified antibody specific for the protein product of any of the marker genes used by the invention.

The term “antibody” also encompasses antigen-binding fragments of an antibody. The term “antigen-binding fragment” of an antibody (or simply “antibody portion,” or “fragment”), as used herein, refers to one or more fragments of a full-length antibody that retain the ability to specifically bind to a polypeptide encoded by one of the marker genes of the invention, or the control reference genes. Examples of binding fragments encompassed within the term “antigen-binding fragment” of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. The antibody is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition”, which as used herein refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody. The antibody can be an intact immuno globulin, e.g., an IgA, IgG, IgE, IgD, IgM or subtypes thereof. The antibody can be conjugated to a functional moiety (e.g., a compound which has a biological or chemical function. The antibody of the invention interacts with a polypeptide, encoded by one of the marker genes of the invention, with high affinity and specificity.

Where the detection molecule is an antibody, the expression of any of the marker genes may be determined according to a specific embodiment, using an immunoassay such as for example, an ELISA, a RIA, a slot blot, a dot blot, immunohistochemical assay, FACS, a radio-imaging assay or a Western blot. It should be noted that any combination of these assays may be also applicable.

Immuno-assays for a protein of interest typically comprise incubating a biological sample of a detectably labeled antibody capable of identifying a protein of interest, and detecting the bound antibody by any of a number of techniques well-known in the art.

As discussed in more detail, below, the term “labeled” can refer to direct labeling of the antibody via, e.g., coupling (i.e., physically linking) a detectable substance to the antibody, and can also refer to indirect labeling of the antibody by reactivity with another reagent that is directly labeled. Examples of indirect labeling include detection of a primary antibody using a fluorescently labeled secondary antibody.

It should be appreciated that all the detecting molecules (either nucleic acid based or amino acid based) used by any of the compositions of the invention are isolated and/or purified molecules. As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95% pure) of non-nucleotide material naturally associated with it, and thus is distinguished from isolated chromosomes. As used herein, the terms “isolated” and “purified” in the context of a proteinaceous agent (e.g., a peptide, polypeptide, protein or antibody) refer to a proteinaceous agent which is substantially free of cellular material and in some embodiments, substantially free of heterologous proteinaceous agents (i.e. contaminating proteins) from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous proteinaceous agent (e.g. protein, polypeptide, peptide, or antibody; also referred to as a “contaminating protein”). When the proteinaceous agent is recombinantly produced, it is also preferably substantially free of culture medium, i.e. culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. Preferably, proteinaceous agents disclosed herein are isolated.

As used herein the term “product of the marker gene” or “products of the marker genes of the invention” refers to the RNA and/or the protein expressed by the marker gene of the invention. In the case of RNA it refers to the RNA transcripts transcribed from the marker gene of the invention. In the case of protein it refers to proteins translated from the genes corresponding to the marker gene of the invention. The “RNA product of a marker gene of the invention” includes mRNA transcripts, and/or specific spliced variants of mRNA whose measure of expression can be used as a marker gene in accordance with the teachings disclosed herein. The “protein product of a marker gene of the invention” includes proteins translated from the RNA products of the marker genes of the invention.

As shown by the following examples, samples obtained from carriers of mutations in at least one of BRCA1 and BRCA2 genes exhibit differential expression of at least one of said marker genes as compared to control samples obtained from non-carrier subjects. Therefore, the composition of the invention may be used for detecting carriers of BRCA1 and BRCA2 gene mutations. Thus, the invention further provides a diagnostic composition for the detection of at least one mutation in at least one of BRCA1 and BRCA 2 genes in a biological sample of a mammalian subject. This particular diagnostic composition comprises at least one isolated oligonucleotide or a collection of at least two isolated detecting oligonucleotides which specifically hybridizes to a nucleic acid sequence of RNA products of at least one marker gene or a collection of at least two marker genes. More specifically, such marker genes may be selected from the group consisting of RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6orf111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4.

It should be noted that these marker genes were shown by the invention as exhibiting a differential expression in lymphocytes from samples obtained from BRCA1 or BRCA2 carriers under irradiation stress. Differential expression of at least one of the marker genes of the invention as compared to a control sample or alternatively, a control non-carrier population or predetermined values of expression that characterize non-carrier population, reflects the existence of at least one mutation in any one of BRCA1 and BRCA2 and may therefore be indicative of an increased genetic predisposition of said subject to a cancerous disorder, disease or condition associated with mutations in any one of BRCA1 or BRCA2.

According to one specific embodiment, the invention provides a diagnostic composition for the detection of at least one mutation of BRCA1 gene in a biological sample of a subject. This particular diagnostic composition comprises at least one isolated oligonucleotide or a collection of at least two isolated oligonucleotides which specifically hybridizes to a nucleic acid sequence of RNA products of at least one marker gene or a collection of at least two marker genes selected from the group consisting of AUH, AU RNA binding protein/enoyl-Coenzyme A hydratase; RGS16, regulator of G-protein signaling 16; MARCH7, membrane-associated ring finger (C3HC4) 7; DNAJC12, DnaJ (Hsp40) homolog, subfamily C, member 12; IFI44L, interferon-induced protein 44-like; SARS, seryl-tRNA synthetase; and SMURF2, SMAD specific E3 ubiquitin protein ligase 2.

It should be further appreciated that in case of detection of BRCA1 mutation, the marker gene may be selected from even a larger group of genes demonstrated by the invention as having most consistent gene expression patterns among all the samples. These genes are represented by genes 1 to 16 of the list disclosed by Table 2. In yet another embodiment, marker genes for BRCA1 gene mutations may be selected form genes exhibiting differential expression of about 1.5 folds. Such genes may be selected from any of the genes set forth in Table 5.

In yet another alternative specific embodiment, the invention provides a composition for the detection of at least one mutation of BRCA2 gene in a biological sample of said subject. This particular composition comprises at least one isolated oligonucleotide or a collection of at least two isolated oligonucleotides which specifically hybridizes to a nucleic acid sequence of RNA products of at least one marker gene or a collection of at least two marker genes selected from the group consisting of RAB3GAP1, RAB3 GTPase activating protein subunit 1 (catalytic); NFAT5, nuclear factor of activated T-cells 5, tonicity-responsive; MRPS6, mitochondrial ribosomal protein S6; MID1IP1, MID1 interacting protein 1 (gastrulation specific G12 homolog (zebrafish)); MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; SFRS18 (C6orf111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D.

It should be further appreciated that in case of detection of BRCA2 mutations, the marker gene may be selected from even a larger group of genes demonstrated by the invention as having most consistent gene expression patterns among all the samples. These genes are represented by genes 17 to 37 of the list disclosed by Table 2. In yet another embodiment, marker genes for BRCA2 gene mutations may be selected form genes exhibiting differential expression of about 2 folds. Such genes may be selected from any of the genes set forth in Table 6.

Some of the invention's particular embodiments describe the composition for the detection of at least one mutation in at least one of BRCA1 and BRCA2 genes in a biological test sample of a mammalian subject, as comprising isolated detecting oligonucleotides, with each oligonucleotide specifically hybridizing to a nucleic acid sequences of RNA products of at least one of at least six marker genes selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6orf111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12, as set forth in Table 4. The aforementioned detecting oligonucleotide molecules are used for determining the level of expression of at least six marker genes in the test sample. As shown in Table 5, a differential expression of at least six such genes in the test sample as compared to a control population is indicative of at least one mutation in at least one of BRCA1 and BRCA2 genes in the subject, and thereby of an increased genetic predisposition of the subject to a cancerous disorder associated with mutations in any one of BRCA1 and BRCA2 genes.

In another embodiment, the composition for the detection of at least one mutation in at least one of BRCA1 and BRCA2 genes in a biological test sample of a mammalian subject comprises isolated detecting oligonucleotides. These oligonucleotide specifically hybridize to nucleic acid sequences of RNA products of at least one of at least six marker genes selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6ORF111), splicing factor, arginine/serine-rich 18, as shown by the eighteen marker genes in Table 7. The detecting oligonucleotide molecules are used for determining the level of expression of at least six marker genes in a sample. It should be further noted that a differential expression of at least six such genes in the test sample as compared to a control population is indicative of at least one mutation in at least one of BRCA1 and BRCA2 genes in the subject, and thereby of an increased genetic predisposition of the subject to a cancerous disorder associated with mutations in any one of BRCA1 and BRCA2 genes.

Still another embodiment the composition for the detection of at least one mutation in at least one of BRCA1 and BRCA2 genes in a biological test sample of a mammalian subject, comprises isolated detecting oligonucleotides, each oligonucleotide specifically hybridizes to a nucleic acid sequences of RNA products of at least one of the at least six marker genes. According to this particular embodiment, said at least six marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2, as set forth in Table 8. It should be noted that the detecting oligonucleotide molecules are used for determining the level of expression of the at least six marker gene in a sample. It should be further noted that a differential expression of at least six of the marker genes in the test sample as compared to a control population is indicative of at least one mutation in at least one of BRCA1 and BRCA2 genes in the subject, and thereby of an increased genetic predisposition of the subject to a cancerous disorder associated with mutations in any one of BRCA1 and BRCA2 genes.

As indicated above, the diagnostic compositions of the invention are specifically used for detection of at lease one mutation in any one of BRCA1 and BRCA2 genes and comprise a nucleic acid based detection molecule. According to this embodiment, the expression of the marker genes may be determined using a nucleic acid amplification assay selected from the group consisting of a Real-Time PCR, microarrays, PCR, in situ Hybridization and Comparative Genomic Hybridization.

According to another specific embodiment, the composition of the invention may comprise detecting molecules specifically adopted for Real Time PCR assay as described herein before.

It should be further appreciated that these specific diagnostic compositions of the invention may alternatively comprise an amino-acid based detecting molecules, for example, an isolated antibody. In such case, the expression of the marker genes may be determined by immuno assays, as described above.

According to a specific embodiment, the diagnostic composition of the invention may be used for detecting at least one mutation in any one of BRCA1 and BRCA2 genes. Existence of mutations in any of these genes may be indicative of an increased genetic predisposition of a subject to a cancerous disorder associated with mutations in any one of BRCA1 and/or BRCA2. According to another embodiment, this cancerous disorder may be breast, ovarian, pancreas or prostate carcinoma. More specifically, such carcinoma may be any one of breast carcinoma and ovarian carcinoma.

Thus, according to another embodiment, the composition of the invention may be applicable for detection, and preferably for early detection of breast cancer. Breast cancer is a cancer of the glandular breast tissue. Worldwide, breast cancer is the fifth most common cause of cancer death (after lung cancer, stomach cancer, liver cancer, and colon cancer). In 2005, breast cancer caused 502,000 deaths (7% of cancer deaths; almost 1% of all deaths) worldwide. Among women worldwide, breast cancer is the most common cancer. It should be indicated that pathological and clinical categories of breast cancer are encompassed by the invention and include ductal carcinoma (65-90%), Lobular carcinoma 10%, Inflammatory breast cancer, Medullary carcinoma of the breast, Colloid carcinoma, Papillary carcinoma and Metaplastic carcinoma.

Early breast cancer can in some cases present as breast pain (mastodynia) or a painful lump. Since the advent of breast mammography, breast cancer is most frequently discovered as an asymptomatic nodule on a mammogram, before any symptoms are present. A lump under the arm or above the collarbone that does not go away may be present. When breast cancer associates with skin inflammation, this is known as inflammatory breast cancer. In inflammatory breast cancer, the breast tumor itself is causing an inflammatory reaction of the skin, and this can cause pain, swelling, warmth, and redness throughout the breast. Changes in the appearance or shape of the breast can raise suspicions of breast cancer.

Another reported symptom complex of breast cancer is Paget's disease of the breast. This syndrome presents as eczematoid skin changes at the nipple, and is a late manifestation of an underlying breast cancer.

Most breast symptoms do not turn out to represent underlying breast cancer. Benign breast diseases such as fibrocystic mastopathy, mastitis, functional mastodynia, and fibroadenoma of the breast are more common causes of breast symptoms. The appearance of a new breast symptom should be taken seriously by both patients and their doctors, because of the possibility of an underlying breast cancer at almost any age.

Occasionally, breast cancer presents as metastatic disease, that is, cancer that has spread beyond the original organ. Metastatic breast cancer will cause symptoms that depend on the location of metastasis.

Moreover, it should be noted that each marker gene of the present invention, is described herein as a marker for detection of carriers of BRCA1 or BRCA2 gene mutations, and therefore may be regarded as a potential marker for breast cancer. The marker genes of the invention might optionally be used alone or in combination with one or more other breast cancer marker genes described herein, and/or in combination with known markers for breast cancer, including but not limited to Calcitonin, CA15-3 (Mucin 1), CA27-29, TPA, a combination of CA 15-3 and CEA, CA 27.29 (monoclonal antibody directed against MUC1), Estrogen 2 (beta), HER-2 (c-erbB2), and/or in any combination thereof.

It should be therefore appreciated that in certain embodiments, where at least six marker genes are used, these marker genes may be also combined with one or more other breast cancer marker genes described herein, and/or in combination with known markers for breast cancer indicated above.

In yet another embodiment, the compositions of the invention may be applicable for the diagnosis of ovarian carcinoma. Ovarian cancer is the most common cause of cancer death from gynecologic tumors in the United States. Early disease causes minimal, nonspecific, or no symptoms. Therefore, most patients are diagnosed in an advanced stage. Overall, prognosis for these patients remains poor. Standard treatment involves aggressive debulking surgery followed by chemotherapy.

Ovarian carcinoma can spread by local extension, lymphatic invasion, intraperitoneal implantation, hematogenous dissemination, and transdiaphragmatic passage. Intraperitoneal dissemination is the most common and recognized characteristic of ovarian cancer. Malignant cells can implant anywhere in the peritoneal cavity but are more likely to implant in sites of stasis along the peritoneal fluid circulation.

It should be noted that in some embodiments, the marker genes of the invention or any polypeptides and/or polynucleotides derived therefrom may be used in the diagnosis of ovarian cancer, alone or in combination with one or more polypeptides and/or polynucleotides of this invention, and/or in combination with known markers for ovarian cancer, including but not limited to CEA, CA125 (Mucin 16), CA72-4TAG, CA-50, CA 54-61, CA-195 and CA 19-9 in combination with CA-125, and/or in combination with the known protein(s) associated with the indicated polypeptide or polynucleotide, as described herein.

According to another embodiment, the diagnostic composition of the invention may be used for detection of prostate carcinoma. Prostate cancer is an important growing health problem, presenting a challenge to urologists, radiologists, and oncologists. Prostate cancer is the most common nondermatologic cancer, yet despite this frequent occurrence, the clinical course is often unpredictable. Most prostate cancers are slow growing and do not manifest themselves during the man's lifetime. Approximately 95% of prostate cancers are adenocarcinomas that develop in the acini of the prostatic ducts. Other rare histopathologic types of prostate cancer occur in approximately 5% of patients, these include small cell carcinoma, mucinous carcinoma, endometrioid carcinoma (prostatic ductal carcinoma), transitional cell carcinoma, squamous cell carcinoma, basal cell carcinoma, adenoid cystic carcinoma (basaloid), signet-ring cell carcinoma, and neuroendocrine carcinoma.

Still further, the composition of the invention may be useful for the diagnosis of pancreatic carcinoma.

Pancreatic cancer is the fourth leading cause of death from cancer in the United States. The disease is slightly more common in men than in women, and risk increases with age.

The cause is unknown, but it is more common in smokers and in obese individuals. There is controversy as to whether type 2 diabetes is a risk factor for pancreatic cancer. A small number of cases are known to be related to syndromes that are passed down through families. Pancreatic cancers can arise from both the exocrine and endocrine portions of the pancreas. Of pancreatic tumors, 95% develop from the exocrine portion of the pancreas, including the ductal epithelium, acinar cells, connective tissue, and lymphatic tissue.

According to certain embodiments of the present invention, any marker gene according to the present invention may optionally be used alone or in combination. Such a combination may optionally comprise a plurality of marker genes described herein, optionally including any sub-combination of marker genes, and/or a combination featuring at least one other marker genes, for example a known marker gene. Furthermore, such a combination may optionally and preferably be used as described above with regard to determining a ratio between a quantitative or semi-quantitative measurement of any marker gene described herein to any other marker gene described herein, and/or any other known marker gene, and/or any other marker. As used herein, “a plurality of” “a collection of” “a combination of” or “a set of” refers to more than two, for example, 3 or more, 4 or more, 5 or more, 6 or more, 7 or more, 8 or more, 9 or more and 10 or more. The present invention thus encompasses any combination of the genes described by Table 4. For example, a combination of 11 or more, 12 or more, 13 or more, 14 or more, 15 or more, 16 or more, 17 or more, 18 or more, 19 or more and 20 or more genes.

According to certain embodiments, the composition of the invention may be used for determining the expression of at least six marker genes. In one particular embodiment, the composition of the invention may be used for determining the expression of at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4, in a biological test sample of a mammalian subject.

In another particular embodiment, the composition of the invention may be used for determining the expression of at least six, at least seven, at least eight, at least nine, at least ten, at least eleven, at least twelve, at least thirteen, at least fourteen, at least fifteen, at least sixteen, at least seventeen, or at least eighteen of the eighteen marker genes listed in Table 7, in a biological test sample of a mammalian subject.

In yet another particular embodiment, the composition of the invention may be used for determining the expression of at least six, at least seven, at least eight, at least nine, at least ten, at least eleven, at least twelve or at least thirteen, of the thirteen marker genes listed in Table 8, in a biological test sample of a mammalian subject.

According to one optional embodiment, the compositions described by the invention or any component thereof, specifically, the detecting molecules may be attached to a solid support. The solid support may include polymers, such as polystyrene, agarose, Sepharose, cellulose, glass, glass beads and magnetizable particles of cellulose or other polymers. The solid-support can be in the form of large or small beads, chips or particles, tubes, plates, or other forms.

A particular and non-limiting example of a diagnostic composition for detecting carriers of BRCA1 and BRCA2 gene mutations, may comprises at least one or a collection of at least two detecting molecules specific for at least one of the marker genes as set forth in Table 4. According to certain embodiments, the diagnostic composition of the invention may comprise detecting molecules specific for at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4.

In another embodiment, the diagnostic composition of the invention may comprise detecting molecules specific for at least six, at least seven, at least eight, at least nine, at least ten, at least eleven, at least twelve, at least thirteen, at least fourteen, at least fifteen, at least sixteen, at least seventeen, or at least eighteen of the eighteen marker genes listed in Table 7.

In yet another embodiment, the diagnostic composition of the invention may comprise detecting molecules specific for at least six, at least seven, at least eight, at least nine, at least ten, at least eleven, at least twelve or at least thirteen, of the thirteen marker genes listed in Table 8.

It should be noted that preferred detecting molecules may be probes and primers derived from these genes. More specifically, such primers and probes are suitable for Real-Time RT-PCR reaction, specifically, the TaqMan® reaction as described by the examples. According to a particularly specific embodiment, such primers and probes may be derived from any of the amplicons as presented by Table 4.

In yet another optional embodiment, any of the compositions of the invention may further comprise at least one detecting molecule or a collection of at least two detecting molecules specific for determination of the expression of at least one control reference gene. Such reference control genes may be for example, RPS9, HSPCB, Eukaryotic 18S-rRNA and β-actin.

Thus, in certain embodiments, the compositions of the invention may further comprise detecting molecules specific for control reference genes. Such genes may be used for normalizing the detected expression levels for each of the marker genes.

The present invention can point at mechanistically-important genes involved with the use of radiation therapy for treating breast cancer. Loss of one allele of BRCA1 leads to impaired repair of double strand breakage (DSB) and sensitivity to ionization caused by irradiation. DSB repair deficiency could lead to cell death by apoptosis. However, haplo-insufficient BRCA1 cells often escape cell death and develop tumors. This may be due to spontaneous hyper-recombination, triggering genome instability [Cousineau, I. and Belmaaza, A. Cell Cycle 6(8):962-971 (2007)]. BRCA1 heterozygous female mice had a higher incidence of ovarian tumors after irradiation without losing the second BRCA1 allele [Jeng, Y. M. et al. Oncogene 26(42):6160-6166 (2007)]. Moreover, reduction in BRCA1 protein impairs homologous recombination (HR) processes [Cousineau, I. and Belmaaza, A. (2007) ibid.], indicating that haplo-insufficiency alone can compromise genome stability and lead to additional cancer-causing mutations. The importance of early and cost-effective detection and diagnosis of carriers of gene mutations in at least one of BRCA1 and BRCA2 by any of the compositions, methods and kits of the invention is thus clear.

Accordingly, in another aspect, the invention relates to a method for the detection of at least one mutation in at least one of BRCA1 and BRCA 2 genes in a biological test sample of a mammalian subject. The method of the invention comprises the steps of: (a) determining the level of at least one marker gene in the test biological sample and optionally, in a suitable control sample. In a particular embodiment, these marker genes may be selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18, splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4. The second step (b) involves determining the level of expression of at least one control gene in the test sample and optionally in a suitable control sample or population. According to a specific embodiment, the control gene may be at least one of RPS9, HSPCB, Eukaryotic 18S-rRNA and β-actin. The third step (c) involves comparing the level of expression as obtained by step (a) of each of the marker genes in the test sample with the level of expression in the control sample or with predetermined expression levels or values of a control non-carrier population; and optionally (d) comparing the level of expression as obtained by step (b) of each of the control reference genes in the test sample with the level of expression in the control sample or with predetermined expression levels or values of a control non-carrier population.

It should be appreciated that the detection of a difference in the level of expression of at least one of the marker genes in the test sample as compared to a control sample according to step (c) may indicate that the test subject is a carrier of at least one mutation in at least one of BRCA1 and BRCA2 genes. Moreover, it should be noted that were control genes are also examined, detection of no difference in the level of expression of the control genes in the test sample as compared to the control sample according to step (d), and a differential expression of the marker genes, even reinforce the indication that the test sample is of a carrier of BRCA1 or BRCA2 gene mutation.

As explained earlier, the inventors have analyzed the marker gene expression values further and discovered specific cutoff values for each gene, a deviation from which of at least six said marker genes is indicative of an increased likelihood of the presence of BRCA1 or BRCA2 mutations in a tested subject. Therefore, another aspect of the invention contemplates a method for the detection of at least one mutation in at least one of BRCA1 and BRCA2 genes in a biological test sample of a mammalian subject, the method comprising the steps of (a) determining the level of expression of at least six marker genes in the test sample and optionally in a suitable control sample. According to specific embodiments, said at least six marker genes may be selected from any one of: (i) a group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2; as set forth in Table 8. Alternatively, these at least six marker genes may be selected from (ii) that is the group of (i) further consisting of: RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6ORF111), splicing factor, arginine/serine-rich 18, as set forth in Table 7. In yet another alternative embodiment, at least six marker genes may be selected from group (iii) that is the group of (i) further consisting of: RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6ORF111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12, as set forth in Table 4. The next step (b), involves determining the level of expression or the expression value of at least one control gene in the test sample and optionally, in a suitable control sample. It should be appreciated that the method of the invention further comprises the step of normalizing the level of expression or the expression value of the marker genes obtained in step (a) with the level of expression of control reference genes obtained in step (b) and thereby obtaining a normalized expression value of each marker gene in the test sample. The next step (c) involves comparing the normalized expression values obtained in steps (a) and (b) of each marker gene in the test sample with a corresponding predetermined cutoff value of each marker gene. The following step (d) involves determining whether the normalized expression value of each marker gene is positive and thereby belongs to a pre-established carrier population or is negative and thereby belongs to a pre-established non-carrier population. The presence of at least six marker genes with a positive normalized expression value indicates that the subject is a carrier of at least one mutation of at least one of BRCA1 or BRCA2 gene.

According to certain embodiments a “positive result” may be determined where a normalized value of a specific marker gene is lower than the cutoff value. In such cases, the specific examined marker gene being down-regulated in the established pre-determined carrier population, and therefore, any normalized value higher than the cutoff value, indicates that the examined sample belongs to non-carrier subject.

According to other alternative embodiments, a normalized value obtained for a specific marker gene that is higher than the cutoff value may be determined as “positive” in case said gene being overexpressed in BRCA1 or BRCA2 mutation carrier population. Therefore, any normalized value that is lower than the cutoff, indicates that said subject belongs to the non-carrier population.

As used herein, the term “expression value”, “level of expression” or “expression level” refers to numerical representation of a quantity of a gene product, which herein is any one of RNA and protein product. For example, gene expression values measured in Real-Time Polymerase Chain Reaction, sometimes also referred to as RT-PCR or quantitative PCR (qPCR), represent luminosity measured in a tested sample, where an intercalating fluorescent dye is integrated into double-stranded DNA products of the qPCR reaction performed on reverse-transcribed sample RNA, i.e., test sample RNA converted into DNA for the purpose of the assay. The luminosity is captured by a detector that converts the signal intensity into a numerical representation which is said expression value, in terms of RNA gene product.

Another example is a microarray RNA assay, where, according to one method, test sample RNA is conjugated to a fluorescent dye and allowed to specifically hybridize with complementary oligonucleotide probes fixed in pre-determined positions on a stationary phase. After excess RNA is washed away, a detector converts the luminosity of each bound fluorescent-dye conjugated RNA species to a numerical representation, which are expression values. There are also various methods for analysis of protein expression values. For example, in some Enzyme-Linked Immunosorbent assay (ELISA) methods, protein samples are incubated in contact with antibodies fixed to a stationary phase and specifically bind a protein of interest. Excess test sample is washed away, and secondary antibodies, conjugated, for example, to a fluorescent dye, are incubated with the protein of interest bound to the fixed specific antibodies. Excess secondary antibody is washed away, and a detector converts the luminosity of the bound secondary antibodies to a numerical representation of the gene expression value, in this case, in terms of protein expression rather than RNA. Examples of expression values are given in Table 9.

It should be noted that a “cutoff value”, sometimes referred to as “cutoff” herein, is a value that meets the requirements for both high diagnostic sensitivity (true positive rate) and high diagnostic specificity (true negative rate). Marker gene expression level values that are higher or lower in comparison with said gene's corresponding cutoff value indicate that the examined sample belongs to a non-carrier or carrier populations, according to the specific criterion for said gene and limited to the said sensitivity and specificity.

Cutoff values may be used as a control sample, said cutoff values being the result of a statistical analysis of marker genes expression value differences in pre-established mutation non-carrier and carrier populations.

The method of calculating a cutoff value is well known in the relevant field. In the case of BRCA1/2 mutations, for example, marker genes expression levels are determined in a large number of BRCA1, BRCA2 or BRCA1 and BRCA2 mutation carriers and non-carrier subjects, the diagnostic sensitivity and diagnostic specificity at each marker gene expression level are determined, and a ROC (Receiver Operating Characteristic) curve is generated on the basis of these values using, for example, a commercially available analytical software program. Then, the marker gene expression level for a diagnostic sensitivity and diagnostic specificity as close to 100% as possible is determined, and this value can be used as the cutoff value that distinguishes between a population of carriers and a population of non-carriers. For example, a diagnostic specificity of the cutoff value for each marker gene may be about 40%, 45%, 50%, 55%, 60%, 65%, 70%, 75%, 80%, 85%, 90%, 95%, 96%, 97%, 98%, 99% and 100%. In another embodiment, the diagnostic sensitivity of the cutoff value for each marker gene may be about 40%, 45%, 50%, 55%, 60%, 65%, 70%, 75%, 80%, 85%, 90%, 95%, 96%, 97%, 98%, 99% and 100%. Non-limiting examples for specificity and sensitivity of cutoff values of the different marker genes of the invention are presented by Table 8.

It should be noted that the terms “sensitivity” and “specificity” are used herein with respect to the ability of one or more marker genes to correctly classify a sample as a carrier sample or a non-carrier sample (a non-carrier sample may be interchangeably referred to as a “normal”, “control”, or “healthy” sample), respectively. “Sensitivity” indicates the performance of the marker genes with respect to correctly classifying carrier samples. “Specificity” indicates the performance of the marker genes with respect to correctly classifying non-carrier samples.

For an illustrative example, 84% specificity and 90% sensitivity for a panel of at least six marker genes used to test a set of control and tumor samples indicates that 84% of the control samples were correctly classified as control non-carrier samples by the panel, and 90% of the carrier sample were correctly classified as carrier samples by the panel.

It should be further noted that, for example, it is possible to determine the diagnostic efficiency (ratio of the sum of the number of true positive cases and the number of true negative cases to the total number of all cases examined) at each detected expression level, and use the expression level for the highest diagnostic efficiency as a cutoff value. In a particular embodiment presented by Example 5 below, cutoff values for thirteen of the marker genes were established between non-carrier subjects and carriers of mutations in BRCA1, BRCA2 or both as presented in Table 8.

As indicated above, the measured levels of expression of each of the examined marker genes are routinely normalized using data of expression levels of the control reference genes. The term “normalization” as used herein refers to any process that makes something more normal, which typically means returning from some state of abnormality. In general scientific context, normalization is a process by which a measurement raw data is converted into data that may be directly compared with other so normalized data. In the context of the present invention, measurements of marker genes expression levels are prone to errors caused by, for example, unequal degradation of measured samples, different loaded quantities per assay and other various errors. To overcome these errors, expression levels for control, stably expressed genes, are measured from the same sample from which the marker gene expression data is extracted. The marker gene expression value is divided by the control gene expression value yielding a normalized marker gene expression value, which is, in fact, marker gene expression value per control gene expression value. Since control gene expression values are equal in different samples, they constitute a common reference point that is valid for such normalization.

The term “ROC” or “Receiver Operator Characteristic” as used herein refers to a receiver operating characteristic (ROC), or simply ROC curve, a graphical plot of the sensitivity versus (1-specificity) for a binary classifier system as its discrimination threshold is varied. The same graph can also be represented equivalently by plotting the fraction of true positives versus the fraction of false positives. ROC analysis provides tools to select possibly optimal threshold values for binary discrimination and to discard suboptimal ones. In the context of this invention, ROC is used to select a cutoff value for marker genes expression levels, a deviation from which indicating a specific likelihood for the presence of at least one of BRCA1 and BRCA2 gene mutations is a tested subject with optimal specificity and sensitivity.

The term “area under the curve” or “AUC” as used herein refers to a ROC statistic or measure which can be interpreted as the probability that, for a specific test, when one randomly picks one positive and one negative example, the classifier, or specific tested marker gene cutoff in this invention, will assign a higher score (indicating a carrier of at least one of BRCA1 and BRCA2 gene mutations) to the positive example than to the negative. High AUC values are herein interpreted as better providing correct diagnosis of BRCA1 and BRCA2 mutations in a tested subject.

Thus, the term “specificity” as used herein refers to the proportion of BRCA1/2 mutations non-carrier subjects which are correctly identified (e.g. the percentage of normal, non-carrier subjects of at least one of BRCA1 and BRCA2 gene mutations, who are identified as not carrying the mutation). Conversely, the term “sensitivity” as used herein refers to the proportion of actual positive i.e., carriers of at least one of BRCA1 and/or BRCA2 gene mutations which are correctly identified as such (e.g. the percentage of carriers of at least one of BRCA1 and BRCA2 gene mutations who are identified as carrying the mutation). It should be noted that the term “BRCA1/2” indicates at least one of BRCA1, BRCA2 or both.

The term “positive predictive value” as used herein refers to the proportion of test subjects with positive test results (i.e. diagnosed as carriers of at least one of BRCA1 and BRCA2 gene mutations) that are correctly diagnosed. Conversely, the “negative predictive value” as used herein is the proportion of test subjects with negative test results who are correctly diagnosed.

It is important to note that the specific group of genes selected herein for detection of carriers of mutations in BRCA1 and/or BRCA2 was defined through a multi-stage stringent process of filtration and validation, which included a microarray analysis, three statistical filtration steps, repeated RT-PCR analysis, and validation of the results, thus ensuring high confidence and reproducibility. Initially, a microarray analysis was used to identify differentially-expressed genes in irradiated versus non-irradiated lymphocytes isolated from nine proven unaffected carriers of BRCA1, eight BRCA2 carriers and from ten non-carrier healthy women. MAS 5.0 and RMA algorithm were used to provide a baseline expression level and detection for each probe set. For each probe set, the ratio between expression level of the BRCA1 and/or BRCA2 mutation carriers and control samples was calculated and finally, ANOVA analysis was used to single out the statistically-significant (p≦0.05) differentially-expressed genes. 137 probe sets in BRCA1 and 1345 probe sets in BRCA2 mutation carriers were so chosen. Intriguingly, the expression patterns in the tested BRCA2 mutation group were highly conserved among all samples, while BRCA1 mutation carriers showed greater heterogeneity in gene expression than in BRCA2 carriers. This could be explained by the numerous biological functions of the BRCA1 protein.

The results set forth in the Examples herein are slightly incongruous with the results of a previous study aimed at BRCA1 and/or BRCA2 genotype prediction by expression profiling in fibroblasts using spotted microarray technology [Kote-Jarai, Z. et al. Clin. Cancer Res. 12(13):3896-901 (2006)]. That study inversely demonstrated a more consistent pattern of gene expression in the BRCA1 mutation carrier group. This apparent discrepancy could be explained by different molecular responses to the same injury agents in different tissues (i.e. fibroblasts vs. lymphocytes). Additionally, the previous study was based on expression analysis using spotted oligonucleotide microarrays, while in this study the Affymetrix microarray platform was used; and different microarray systems are not always comparable to each other [Hardiman, G. Pharmacogenomics 5(5): 487-502 (2004)].

Next, several statistical filters were applied to the results. The first filter adjusted for a 5% false discovery rate, which left 596 differentially-expressed genes for BRCA2 carriers (but could not be applied to differentially-expressed genes of BRCA1 carriers). Of the filtered genes, those with a minimum of a two-fold expression difference between the BRCA1 or BRCA2 groups and the control groups were selected, creating a set of 86 genes in BRCA1 carriers and 97 genes in BRCA2 carriers. Next, genes with the most reproducible pattern of expression in all samples within the same group were selected. This process resulted in a list of a total of 38 genes. The filtered results were subsequently analyzed using RT-PCR, a reliable quantitative technique considered the golden standard of RNA semi-quantitation. In this analysis, seventeen samples in the BRCA1 group, ten from the BRCA2 group and twelve samples of non-carriers of mutations were assayed. Five known housekeeping genes which were similarly expressed in the three groups were served as internal controls. In total, forty three genes were tested by TaqMan® gene cards RT-PCR. Of those, twenty genes were expressed differentially in a statistically-significant (p<0.05) manner. The eighteen genes that demonstrated the most significant differential expression were chosen for validation and the calculated expression cutoff values for the chosen genes were assessed for diagnostic value. Lymphocytes from twenty-one female carriers of BRCA1, BRCA2, or both and 19 non-carriers were isolated, and the differential expression of the filtered marker gene group was assayed in irradiated versus non-irradiated cells. Through the use of a ROC curve analysis of the results obtained in said validation experiment, a further five candidate marker genes were discarded. Furthermore, the ROC analysis provided standardized expression threshold values representing control sample marker gene expression values that allowed comparison of the expression values of the test sample marker genes with said threshold values and demonstrated that the accumulation of at least six positive results as compared to said thresholds from the thirteen rigorously-filtered marker genes indicated the presence of a BRCA1 and BRCA2 mutation or mutations with a sensitivity of about 75% to 100%, more specifically 80% to 98%, more specifically 85% to 96%, more specifically 87% to 94%, more specifically 89% to 92%, particularly any one of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 99.9% sensitivity, and a specificity of about 65% to 100%, more specifically 70% to 98%, more specifically 75% to 96%, more specifically 80% to 94%, more specifically 82% to 92%, particularly any one of 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 99.9%. In a specific illustrative example, the sensitivity may be about 90% and specificity may be about 84%.

It is interesting to note that according to a Gene Ontology analysis, the most differently expressed genes from BRCA1/2 mutation groups are related to a small number of GO terms, namely: regulation pathways, DNA repair processes, cell cycle regulation and cancer. STAT5 funnels extracellular signals of cytokines, hormones, and growth factors into transcriptional activity in the mammary gland. It causes tumorigenesis, but delays metastasis progression. Consequently, STAT5 activity in breast-cancer specimens marks a better prognosis for survival [Barash, I. J. Cell Physiol. 209(2):305-313 (2006)], and its radiation-induced increase likes notes a protective feedback reaction. Another gene, RPS6KB1, a kinase, has been shown to be overexpressed in some breast cancer cell lines [Sinclair, C. S. et al., Breast Cancer Res. Treat. 78(3):313-22 (2003)].

The analysis results provide an additional insight towards the role of the biological effect of heterozygous mutations in BRCA1 and BRCA2 genes in cellular response to irradiation DNA damage and constitute a molecular functional tool that can be used to predict the presence of BRCA1/2 mutations in individual in a sensitive, simple, inexpensive and easily obtained fashion.

As used herein in this specific embodiment, the term “marker gene” refers to a gene that is differentially regulated between a carrier or a population of carriers of mutations in any one of BRCA1 or BRCA2 genes and a non-carrier individual or a population of non-carriers.

“Differentially expressed” can also include a measurement of the RNA or protein encoded by the marker gene of the invention in a sample or plurality of samples as compared with the amount or level of RNA or protein expression in a second sample or population or plurality of samples, specifically, a control sample of non-carrier subject. Differential expression can be determined as described herein and as would be understood by a person skilled in the art. The term “differentially expressed” or “changes or difference in the level of expression” refers to an increase or decrease in the measurable expression level of a given marker gene as measured by the amount of RNA and/or the amount of protein in a sample as compared with the measurable expression level of a given marker gene in a second sample, specifically, a control sample. The term “differentially expressed” or “changes or differences in the level of expression” can also refer to an increase or decrease in the measurable expression level of a given marker gene in a population of samples as compared with the measurable expression level of a marker gene in a second population of samples, for example, a control sample obtained from a non-carrier subject. As used herein, “differentially expressed” can be measured using the ratio of the level of expression of a given marker gene(s) as compared with the mean expression level of the given marker gene(s) of a control sample wherein the ratio is not equal to 1.0. Differentially expressed can also be measured using p-value. When using p-value, a marker gene is identified as being differentially expressed as between a first and second population when the p-value is less than 0.1. More preferably the p-value is less than 0.05. Even more preferably the p-value is less than 0.01. More preferably, the p-value is less than 0.005. Most preferably, the p-value is less than 0.001. When determining differentially expression on the basis of the ratio, an RNA or protein is differentially expressed if the ratio of the level of expression in a first sample as compared with a second sample is greater than or less than 1.0. For example, a ratio of greater than 1.2, 1.5, 1.7, 2, 3, 4, 5, 6, 7, 8, 9, 10, 20 or a ratio less than 1, for example 0.8, 0.6, 0.4, 0.2, 0.1. 0.05. In another specific embodiment of the invention, a nucleic acid transcript is differentially expressed if the ratio of the mean of the level of expression of a first population as compared with the mean level of expression of the second population is greater than or less than 1.0. For example, a ratio of greater than 1.2, 1.5, 1.7, 2, 3, 4, 5, 6, 7, 8, 9, 10, 20, or a ratio less than 1, for example 0.9, 0.8, 0.6, 0.4, 0.3, 0.2, 0.1, 0.05 or 0.01. In another embodiment of the invention, a nucleic acid transcript is differentially expressed if the ratio of its level of expression in a first sample as compared with the mean of the second population is greater than or less than 1.0 and includes for example, a ratio of greater than 1.2, 1.5, 1.7, 2, 3, 4, 5, 6, 7, 8, 9, 10, 20, or a ratio less than 1, for example 0.9, 0.8, 0.6, 0.4, 0.3, 0.2, 0.1, 0.05 or 0.01.

More specifically, “Differentially increased expression” or “up regulation” refers to genes which demonstrate at least 10% or more, for example, 20%, 30%, 40%, or 50%, 60%, 70%, 80%, 90% or more, or 1.1 fold, 1.2 fold, 1.4 fold, 1.6 fold, 1.8 fold, or more increase in gene expression (as measured by RNA expression or protein expression), relative to a control sample.

“Differentially decreased expression” or “down regulation” refers to genes which demonstrate at least 10% or more, for example, 20%, 30%, 40%, or 50%, 60%, 70%, 80%, 90% or a less than 1.0 fold, 0.8 fold, 0.6 fold, 0.4 fold, 0.2 fold, 0.1 fold or less decrease in gene expression (as measured by RNA expression or protein expression), relative to a control.

It should be further noted that in case the expression level of more than one marker gene is examined by the diagnostic method of the invention, it may reflect and result in “gene expression pattern” or “gene expression profile” of the diagnosed individual. As used herein, a “gene expression pattern” or “gene expression profile” indicates the combined pattern of the results of the analysis of the level of expression of at least one, preferably, at least two or more marker genes of the invention including 3, 4, 5, 6, 7, 8, 9, 10, 11, 12, 13, 14, 15, 16, 17, 18, 19, 20 or more or all of the markers of the invention. More specifically, at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4. In another embodiment, at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen or at least eighteen of the eighteen marker genes listed in Table 7. According to another embodiment, at least one, at least two, at least three, at least four, at least five, at least six, at least seven, at least eight, at least nine, at least ten, at least eleven, at least twelve or at least thirteen, of the thirteen marker genes listed in Table 8. According to one particular embodiment, “gene expression profile” indicates the combined pattern of the results of the analysis of the level of expression of at least six marker genes selected from the group of marker genes indicated in any one of Tables 4, 7 or 8. A gene expression pattern or gene expression profile can result from the measurement of expression of the RNA or protein products of the marker genes of the invention and can be done using any known technique. For example, techniques to measure expression of the RNA products of the marker genes of the invention includes, PCR based methods (including RT-PCR) and non PCR based method as well as micro-array analysis. To measure protein products of the marker genes of the invention, techniques include western blotting and ELISA analysis.

More particularly, according to this embodiment, determination of the expression of the marker genes as well as of the control genes may be performed by the following steps:

The first step (i) involves providing an array comprising:

(A) at least one detecting nucleic acid or amino acid molecule specific for determination of the expression of at least one of said marker genes. The detecting molecule may be a set of primers, a probe or both or alternatively or additionally, an antibody. It should be noted that each of said detecting molecules is located in a defined position in said array. This array further comprise (B) at least one detecting nucleic acid or amino acid molecule specific for determination of the expression of at least one of the control genes. Each of the detecting molecules is located in a defined position in the array.

The second step (ii), involves contacting aliquots of the test sample and particularly, nucleic acids (RNA samples) or protein product prepared from the irradiated lymphocytes, and aliquots of the control samples with the detecting molecules (primers, probes or both or antibodies) comprised in said array of (i) under conditions allowing for detection of the expression of the marker genes and the control genes in both the test and the optional the control samples. The third step (iii), involves determining the level of the expression of the marker genes and the control genes in the test and in the optional control samples by suitable means. Preferably, by Real Time-PCR or micro-arrays, as indicated in detail herein before.

As stated earlier, gene expression “profiles” or “patterns” have indeed been found by the inventors to correlate with the presence of BRCA11 and/or 2 mutations in subjects. Hence, the method of detecting such subjects can be materialized in various ways, for example, in a specific embodiment, the determination of the level of expression of at least six of the marker genes according to step (a) and of at least one of the control gene according to step (b), in a test sample and optionally in a control sample is performed by a method comprising the steps of: (I) providing an array comprising: (A) detecting molecules specific for determining the expression of at least six of the marker genes, where each of the detecting molecules is located in a defined position in the array, and the detecting molecules are selected from isolated detecting nucleic acid molecules and isolated detecting amino acid molecules; and (B) at least one detecting molecule specific for determination of the expression of at least one of the control genes, where each of the detecting molecules is located in a defined position in the array and where the detecting molecule is selected from isolated detecting nucleic acid molecule and isolated detecting amino acid molecule. The second step (II) involves contacting aliquots of the test sample or any nucleic acid or amino acid product obtained therefrom, and optionally, aliquots of the control sample or any nucleic acid or amino acid product obtained therefrom with the detecting molecules comprised in the array of (I) under conditions allowing for detection of the expression of at least six marker genes and the control genes in the test and optionally, in the control samples. Finally, step (III) determining the level of the expression of the at least six marker genes and of at least one control gene in the test and optionally, control samples contacted with detecting molecules comprised in the array of (I) by suitable means.

In a specific mode of embodiment of the present invention, carriers of mutations in BRCA1 and BRCA2 can be distinguished from each other by previously establishing marker genes cutoff values and comparing the detected marker gene expression level values to said cutoff values.

As indicated above, the different detecting molecules of the invention are provided attached, comprised, or connected to an array. The term “array” as used by the methods and kits of the invention refers to an “addressed” spatial arrangement of the detecting molecules specific for the marker genes of (A) and, the detecting molecules specific for the control genes of (B). Each “address” of the array is a predetermined specific spatial region containing a detecting molecule. For example, an array may be a plurality of vessels (test tubes), plates (or even different predetermined locations in one plate or one slide, micro-wells in a micro-plate each containing a different detecting molecule. An array may also be any solid support holding in distinct regions (dots, lines, columns) different detecting molecules. The array preferably includes built-in appropriate controls, for example, regions without the sample, regions without any detecting molecules, regions without either, namely with solvent and reagents alone. Solid support used for the array of the invention will be described in more detail herein after, in connection with the kits provided by the invention.

Reference to “determining” as used by the methods of the present invention, includes estimating, quantifying, calculating or otherwise deriving a level of expression of the marker or control genes by measuring an end point indication that may be for example, the appearance of a detectable product.

It should be appreciated that the detection step may be performed using the tested sample as obtained from the tested subject, or alternatively, may be performed using any constituent or material derived or prepared therefrom. As a non-limiting example, it should be noted that the method of the invention further encompasses the use of nucleic acid molecules and or proteins prepared from the tested sample.

Thus, according to one preferred embodiment the detecting molecule used for the diagnostic method of the invention may be an isolated nucleic acid molecule or an isolated amino acid molecule, or any combination thereof.

According to one alternative and specific embodiment, the method of the invention uses as detecting molecules isolated nucleic acid molecules. More specifically, such nucleic acid molecule may be an isolated oligonucleotide which specifically hybridizes to a nucleic acid sequence of the RNA products of at least one marker gene selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6ORF111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4.

In another specific embodiment, the detecting nucleic acid molecules are isolated oligonucleotides and each oligonucleotide specifically hybridizes to a nucleic acid sequence of the RNA products of at least one of the at least six marker genes or of at least one of the control genes.

It should be appreciated that further genes may serve as marker genes by the method of the invention. For example, any of the genes demonstrating a significant differential expression listed in Table 2.

Accordingly, the detecting molecule specific for the control reference genes may be therefore an isolated nucleic acid molecule, and preferably, an isolated oligonucleotide which specifically hybridizes to a nucleic acid sequence of the RNA products of at least one control reference gene. Examples for possible control genes may be RPS9, HSPCB, Eukaryotic 18S-rRNA and β-actin.

According to a specifically preferred embodiment, the oligonucleotide used as a detecting molecule by the method of the invention may be for example, a pair of primers, a nucleotide probe or any combination thereof.

In a specific embodiment, the primers and probes used by the method of the invention may be selected from the amplicons defined by Table 4. Nevertheless, it should be appreciated that any region of such marker genes may be used as an amplicon and therefore as a possible region for targeting primers and probes.

Accordingly, the expression of the marker gene and of the control reference gene may be determined according to a preferred embodiment, using a nucleic acid amplification assay such as Real Time PCR, micro arrays, PCR, in situ Hybridization and Comparative Genomic Hybridization, as described in detail herein before.

According to an alternative embodiment, the method of the invention uses an isolated amino acid molecule as the detecting molecule. Such detecting molecule may be therefore an isolated polypeptide which binds selectively to the protein product of at least one marker gene selected from the group consisting of RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6ORF111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4.

Accordingly, the detecting molecule for the control reference genes may be an isolated polypeptide which binds selectively to a protein product of at least one control reference gene. For example, RPS9, HSPCB, Eukaryotic 18S-rRNA and β-actin.

According to a specifically preferred embodiment, the detecting molecule used by the method of the invention, may be an isolated antibody and the marker genes expression may be determined using an immunoassay selected from the group consisting of an ELISA, a RIA, a slot blot, a dot blot, immunohistochemical assay, FACS, a radio-imaging assay or a Western blot, as described herein before.

In yet another embodiment, the isolated detecting amino acid molecules are isolated antibodies, and each antibody binds selectively to a protein product of at least one of the at lest six marker genes or of the at least one control genes.

According to a particular embodiment, the invention provides a specific method for the detection of at least one mutation of BRCA1 gene in a biological sample of a tested subject. According to this particular embodiment, the marker gene or a collection of at least two marker genes may be selected from the group consisting of: AUH, AU RNA binding protein/enoyl-Coenzyme A hydratase; RGS16, regulator of G-protein signaling 16; MARCH7, membrane-associated ring finger (C3HC4) 7; DNAJC12, DnaJ (Hsp40) homolog, subfamily C, member 12; IFI44L, interferon-induced protein 44-like; SARS, seryl-tRNA synthetase; and SMURF2, SMAD specific E3 ubiquitin protein ligase 2.

It should be further appreciated that in case of detection of BRCA1 mutation, the marker gene may be selected from even a larger group of genes demonstrated by the invention as having most consistent gene expression patterns among all the samples. These genes are represented by genes 1 to 16 of the list disclosed by Table 2. In yet another embodiment, marker genes for BRCA1 gene mutations may be selected form genes exhibiting differential expression of about 1.5 folds. Such genes may be selected from any of the genes set forth in Table 5.

According to another particular embodiment, the invention provides a specific method for the detection of at least one mutation of BRCA2 gene in a biological sample of a tested subject. According to this particular embodiment, the marker gene or a collection of at least two marker genes may be selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; MID1IP1, MID1 interacting protein 1 (gastrulation specific G12 homolog (zebrafish)); MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; SFRS18 (C6ORF111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D.

It should be further appreciated that in case of detection of BRCA2 mutations, the marker gene may be selected from even a larger group of genes demonstrated by the invention as having most consistent gene expression patterns among all the samples. These genes are represented by genes 17 to 37 of the list disclosed by Table 2. In yet another embodiment, marker genes for BRCA2 gene mutations may be selected form genes exhibiting differential expression of about 2 folds. Such genes may be selected from any of the genes set forth in Table 6.

The present invention relates, in some embodiments, to diagnostic assays, which in some embodiments, utilizes a biological sample taken from a subject (patient or healthy person, in some embodiments or carrier or non-carrier subject), which for example may comprise any biological sample, such as body fluid or secretion including but not limited to blood, blood cells such as lymphocytes, seminal plasma, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, secretions of the breast ductal system (and/or lavage thereof), broncho alveolar lavage, lavage of the reproductive system, lavage of any other part of the body or system in the body, samples of any organ including but not limited to lung, colon, ovarian and/or breast tissue, feaces or a tissue sample, any cells derived therefrom, or any combination thereof. In some embodiments, the term encompasses samples of in vitro or ex vivo cell culture or cell culture constituents. The sample can optionally be diluted with a suitable eluant before contacting the sample with the detecting molecule/s of the invention and/or performing any other diagnostic assay.

As used herein, “patient”, “subject”, “carrier” or “individual” refers to a mammal, preferably human, that is diagnosed by the method of the invention. More specifically, the term “carrier” or “carrier subject” as used herein refers to a person or organism whose genotype includes at least one mutated allele of at least one of BRCA1 and BRCA2. Said mutations may be any one or a combination of deletions, insertions, truncations, rearrangements, antisense or missense mutations or any other modifications that render the products of said genes non-functional. Said carrier may be a symptomatic or an asymptomatic carrier, that is, the carrier may present pathophysiological signs as the result of said mutations, typically in the form of the development of breast cancer and in some cases other cancer types. Carriers have an increased likelyhood of developing cancer, particularly breast cancer. For example, women carriers of an abnormal BRCA1 or BRCA2 gene have up to an 85% risk of developing breast cancer by age 70 and an increased risk of developing ovarian cancer, which is about 55% for women with BRCA1 mutations and about 25% for women with BRCA2 mutations. In addition to breast cancer, mutations in the BRCA1 gene also increase the risk on ovarian, fallopian tube and prostate cancers. Moreover, precancerous lesions (dysplasia) within the Fallopian tube have been linked to BRCA1 gene mutations. Pathogenic mutations anywhere in a model pathway containing BRCA1 and BRCA2 greatly increase risks for a subset of leukemias and lymphomas. The carrier may present any of the above cancer types in the present or in the past, and may also not present any of the above cancer types in the present or in the past. More specifically, according to certain embodiments, a carrier is a non-symptomatic subject that has never presented or developed any proliferative disorder, particularely the cancerous disorders indicated above.

Conversly, the term “non-carrier” or “non-carrier subject” as used herein refers to a person or organism whose genotype does not include at least one mutated allele of at least one of BRCA1 and BRCA2. Said mutations may be any one or a combination of deletions, insertions, truncations, rearrangements, antisense or missense mutations or any other modifications that render the products of said genes non-functional. Although non-carriers have a lower likelyhood to develop breast cancer, ovarian, fallopian tube and prostate cancers, precancerous lesions (dysplasia) within the fallopian tube or a subset of leukemias and lymphomas as compared to carriers, said non-carriers may present any of the above cancer types in the present or in the past, and may also not present any of the above cancer types in the present or in the past

According to a particular and specific optional embodiment, where the sample used comprises cells obtained from the tested subject, the method of the invention may comprise an additional step. The additional step includes induction of DNA damage by treating the cells with an agent inducing such damage. This may be performed by exposing the cells to irradiation as demonstrated by the following examples. It should be noted that such additional step may be preferably performed as a preliminary step prior to determination of the expression levels or profile of the marker genes or the control reference genes. Thus, according to a specific embodiment, the sample used for the compositions, methods and kits of the invention are irradiated lymphocytes obtained from a tested subject.

Thus, according to a specific and particular embodiment, the invention provides a method for the detection of at least one mutation in at least one of BRCA1 and BRCA2 genes in a biological test sample of a mammalian subject. According to this embodiment, the diagnostic method comprises the steps of:

(a) providing a nucleic acid sample prepared from lymphocytes of a tested mammalian subject and optionally, a nucleic acid sample obtained from lymphocytes of a suitable control. It should be noted that in order to induce DNA damage, the lymphocytes were irradiated prior to nucleic acid preparation;
(b) determining the level of expression of at least one of the marker genes identified by the invention, in said test sample and optionally, in a suitable control sample.
Step (c) involves determining the level of expression of at least one control gene in said test sample and in a suitable control sample, wherein said at least one control gene may be any one of RPS9, HSPCB, Eukaryotic 18S-rRNA and β-actin;
(d) comparing the level of expression as obtained by step (b) of each of the marker genes in the test sample optionally with the level of expression in the control sample; and (e) comparing the level of expression as obtained by the optional step (c) of each of the control genes in said test sample optionally with the level of expression in the control sample.

It should be appreciated that the expression level of each of the marker genes in the test and optionally in the control sample is normalized by comparing to the levels of the control reference genes.

It should be noted that detecting a difference in the level of expression, or as also indicated by the invention a “differential expression” of at least one of the marker genes in the test sample as compared to the control sample according to step (c), is indicative of that the tested subject is a carrier of at least one mutation in at least one of BRCA1 and BRCA2 genes.

Alternatively, or in addition to comparison of the normalized levels of expression of any of the marker genes in the tested sample to the levels in a pre-determined control non-carrier sample, the levels of expression may be compared to a predetermined value representing each distinguished population. Such values may be represented by a cutoff value for each marker gene, that distinguish between a control non-carrier population and a carrier population. By comparing the normalized expression values obtained for each marker gene to said cutoff value, one can determine if the tested sample is of a carrier or of a non-carrier subject. It should be noted that according to Example 5, “positive” result obtained for at least six marker genes adequately indicates that said sample is of a carrier subject. It should be noted that a “positive result” is in the range of values detected for a predetermined carrier population for each marker gene. A “negative result” is in the range of values detected for a predetermined non-carrier population for each marker gene.

It should be further appreciated, that when the control reference gene are also examined and compared to a non-carrier population, no difference in the level of expression of the control genes is expected when the tested sample is compared to a control sample according to step (d). Therefore, a differential expression in the marker genes and no difference in the expression of the control genes, indicates that the tested subject is a carrier of at least one gene mutation in at least one of BRCA1 and BRCA2.

More particularly, according to this embodiment, determination of the expression of the marker genes and of the control genes may be performed by the following steps:

The first step (i), involves providing an array comprising:

(A) at least one detecting nucleic acid molecule specific for determination of the expression of at least one of said marker genes. The detecting nucleic acid molecule may be a set of primers, a probe or both. It should be noted that each of said detecting molecules is located in a defined position in said array; and optionally,
(B) at least one detecting nucleic acid molecule specific for determination of the expression of at least one of the control genes. Each of the detecting nucleic acid molecules is located in a defined position in the array.

The second step (ii), involves contacting aliquots of the test sample and particularly, nucleic acids (RNA samples) product prepared from the irradiated lymphocytes, and optionally, aliquots of the control sample with the detecting nucleic acid molecules (primers, probes or both) comprised in said array of (i) under conditions allowing for detection of the expression of the marker genes and the control genes in the test and optionally, the control samples; and

The third step (iii), involves determining the level of the expression of the marker genes and the control genes in the test and optional control samples by suitable means. Preferably, by Real Time-PCR or micro-arrays, as indicated in detail herein before.

It should be noted that the resulting expression values measured for each marker gene are normalized with the expression values of a control reference gene to obtain a normalized expression value for each marker gene examined.

It should be further noted that the detection of a carrier of at least one mutation in any one of BRCA1 or BRCA2 genes by the method of the invention may be an indicative of an increased genetic predisposition of the diagnosed subject to a cancerous disorder associated with at least one mutation in at least one of BRCA1 and BRCA2 genes.

Such cancerous disorders may be for example, breast, ovarian, pancreas and prostate carcinoma.

It should thus be appreciated that the method of the invention may provide early detection of such cancerous disorders. Therefore, the invention may be applicable and therefore provides a diagnostic method for the diagnosis, preferably, early detection of breast, ovarian, pancreas and prostate carcinoma, and particularly of breast carcinoma and ovarian carcinoma.

It should be thus noted that this invention may provides diagnostic methods optionally applicable in the selection of a particular therapy, or optimization of a given therapy for a disease, disorder or condition.

To facilitate convenience and ease of use of the aforesaid method, the inventors contemplated a kit for the detection of carriers of at least one mutation in BRCA1 or/and BRCA 2 genes. Thus, another aspect of the present invention contemplates a diagnostic kit comprising:

(a) means for obtaining a sample of a mammalian subject; (b) at least one detecting molecule or a collection of at least two detecting molecules specific for determination of the expression of at least one marker gene or a collection of at least two marker genes selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6ORF111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4. (c) at least one detecting molecule or a collection of at least two detecting molecules specific for determination of the expression of at least one control reference gene or a collection of at least two control reference genes. According to a specific embodiment, these control reference genes may be selected from the group consisting of: RPS9, HSPCB, Eukaryotic 18S-rRNA and β-actin; (d) optionally, at least one control sample that may be at least one of a negative control sample and a positive control sample; (e) instructions for carrying out the detection and quantification of expression of the marker genes and of the control reference gene in the tested sample, and for normalizing the expression values measured for each marker gene with a control reference gene;
(f) instructions for evaluating the differential expression of the marker gene in the tested sample and of a control reference gene in the sample as compared to the expression of the marker gene and the control reference gene in the optional control sample.

It should be noted that the detecting molecule of the marker genes (b) or the control genes (c), may be provided by the kit of the invention attached, connected, embedded, linked, placed, glued or fused to a solid support or to an array, as described herein before.

As used herein, the term “control” or “control sample” includes positive or negative controls. In the context of this invention the term “positive control” refers to one or more samples isolated from an individual or group of individuals who are classified as carrier of mutations in any one of BRCA1 or BRCA2 genes. The term “negative control” refers to one or more samples isolated from an individual or group of individuals who are classified as non-carrier of mutations in any one of BRCA1 or BRCA2 genes.

According to an alternative or additional embodiment, instead of control samples, the kit of the invention may comprise a standard curve/s illustrating the expression of the marker genes and optionally of the control genes in predetermined positive or negative control samples, e.g. values obtained from a population of carriers or values of expression obtained for population of non-carriers.

Thus, as another more specific embodiment, the invention provides a kit comprising:

(a) means for obtaining a sample of a mammalian subject; (b) detecting molecules specific for determining the level of expression of at least six marker genes, wherein the detecting molecules are selected from isolated detecting nucleic acid molecules and isolated detecting amino acid molecules. According to this embodiment, at least six marker genes may be selected from any one of: (i) a group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2, as set forth in Table 8, (ii) the group as defined in (i) further consisting of: RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6ORF111), splicing factor, arginine/serine-rich 18, as set forth in Table 7; (iii) the group as defined in (i) further consisting of: RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6ORF111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12, as set forth in Table 4; (c) at least one detecting molecule specific for determining the expression of at least one control gene; (d) optionally, at least one control sample selected from a negative control sample and a positive control sample; (e) instructions for carrying out the detection and quantification of expression of the at least six marker genes and of at least one control gene in the sample; (f) instructions for evaluating the differential expression of the at least six marker genes and of control genes in the test as compared to the expression of at least six marker genes and optionally control genes in the control sample.

It should be recognized that the levels of expression measured for each marker gene are normalized as indicated herein before, with the levels of expression obtained for the control marker genes. The present kit therefore may also include instructions for such normalization procedure.

In particular embodiments, the kits of the invention may also include cutoff tables, schematic plots, diagrams, software or other means that facilitate the evaluation of each marker gene normalized expression value and conversion of said at least six marker genes normalized expression values into an indication of the presence of at least one mutation in at least one of BRCA1 and BRCA2 in a subject from which the tested sample originates. For example, the kit may include a computer program that manually or automatically receives marker genes and, optionally, control genes expression values, optionally performs normalization, compares the normalized values to pre-determined cutoff values, counts the number of marker genes that exceed the corresponding cutoff values and indicates whether the tested sample originates from a carrier or non-carrier of at least one of BRCA1 and BRCA2 mutations. In another example, the kit includes a colored cutoff table that facilitates the conversion of said at least six marker genes normalized expression values into an indication of the presence of at least one mutation in at least one of BRCA1 and BRCA2 in a subject from which the tested sample originates by easily identifying values above and below specific marker gene cutoff values and providing a summation of the number of marker genes deviating from said cutoffs, thus indicating the presence or absence of said mutations.

According to one embodiment, the detecting molecules comprised within any of the kits of the invention may be isolated nucleic acid molecules or isolated amino acid molecules, or any combination thereof.

According to one specific and preferred embodiment, the detecting molecule comprised within the kit of the invention may be an isolated nucleic acid molecule. Such molecule may be preferably, an isolated oligonucleotide which specifically hybridizes to a nucleic acid sequence of the RNA products of at least one marker gene selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6ORF111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4. It should be noted that the marker genes may be also selected from any of the genes listed in Table 2.

It should be further noted that according to certain embodiments, the isolated detecting nucleic acid molecules provided with the kit of the invention may be isolated oligonucleotides. Each oligonucleotide specifically hybridizes to a nucleic acid sequence of the RNA products of at least one of said at least six marker genes or of at least one of said control gene. It should be further indicated that these at least six marker genes may be selected from the marker genes presented by any one of Tables 4, 7 or 8. According to certain embodiments, the detecting molecules provided by the kits of the invention may be specifically suitable for determining the expression of at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4, in a biological test sample of a mammalian subject.

According to another embodiment, the detecting molecules provided by the kits of the invention may be specifically suitable for determining the expression of at least six, at least seven, at least eight, at least nine, at least ten, at least eleven, at least twelve, at least thirteen, at least fourteen, at least fifteen, at least sixteen, at least seventeen or at least eighteen of the eighteen marker genes listed in Table 7, in a biological test sample of a mammalian subject.

In yet another embodiment, the detecting molecules provided by the kits of the invention may be specifically suitable for determining the expression of at least six, at least seven, at least eight, at least nine, at least ten, at least eleven, at least twelve or at least thirteen, of the thirteen marker genes listed in Table 8, in a biological test sample of a mammalian subject.

Accordingly, the kit of the invention may therefore comprises as the detecting molecule for the control reference genes, an oligonucleotide which specifically hybridizes to a nucleic acid sequence of the RNA products of at least one control reference gene selected from the group consisting of: RPS9, HSPCB, Eukaryotic 18S-rRNA and β-actin.

According to another embodiment, such oligonucleotide may be a pair of primers or nucleotide probe or any combination, mixture or collection thereof.

According to such specific and particular embodiment, the primers and probes used by the kits of the invention may be derived from regions of the genes that are also defined as amplicons (selected regions for amplification). Examples for amplicons used are demonstrated by Table 4, which also discloses partial sequences of the amplicons (SEQ ID NO. 25 to 48) used in the following Examples. It should be appreciated that primers and probes may be derived from any other amplicon in the listed marker genes described by the invention.

According to another optional embodiment, the kits of the invention may further comprise at least one reagent for performing a nucleic acid amplification based assay. Such nucleic acid amplification assay may be any one of PCR, Real Time PCR, micro arrays, in situ Hybridization and Comparative Genomic Hybridization.

According to an alternative embodiment, the detecting molecule comprised within the kits of the invention may be an isolated amino acid molecule, for example, an isolated polypeptide which binds selectively to the protein product of at least one marker gene selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6ORF111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4.

According to specific embodiment, the isolated detecting amino acid molecules provided by the kit of the invention may be isolated antibodies. Each antibody binds selectively to the protein product of at least one of at least six marker genes or of at least one of said control gene. It should be further indicated that these at least six marker genes may be selected from the marker genes presented by any one of Tables 4, 7 or 8. According to certain embodiments, the antibodies provided by the kits of the invention may be specifically suitable for determining the expression of at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4, in a biological test sample of a mammalian subject.

According to another embodiment, the antibodies provided by the kits of the invention may be specifically suitable for determining the expression of at least six, at least seven, at least eight, at least nine, at least ten, at least eleven, at least twelve, at least thirteen, at least fourteen, at least fifteen, at least sixteen, at least seventeen or at least eighteen of the eighteen marker genes listed in Table 7, in a biological test sample of a mammalian subject.

In yet another embodiment, the antibodies provided by the kits of the invention may be specifically suitable for determining the expression of at least six, at least seven, at least eight, at least nine, at least ten, at least eleven, at least twelve or at least thirteen, of the thirteen marker genes listed in Table 8, in a biological test sample of a mammalian subject.

Accordingly, the detecting molecule specific for the control reference genes may be an isolated polypeptide which binds selectively to the protein product of at least one control reference gene selected from the group consisting of RPS9, HSPCB, Eukaryotic 18S-rRNA and β-actin.

In such specific embodiment where the detecting molecule may be an isolated antibody the kits of the invention may optionally further comprise at least one reagent for performing an immuno assay, such as ELISA, a RIA, a slot blot, a dot blot, immunohistochemical assay, FACS, a radio-imaging assay, Western blot or any combination thereof.

According to a preferred embodiment, the kits provided by the invention may further comprise suitable means and reagents for preparing or isolating at least one of nucleic acids and amino acids from the examined sample.

As shown by the following examples, the marker genes of the invention demonstrate a clear differential expression in carries of BRCA1 and/or BRCA2 gene mutations. Thus, the invention further provides a particular kit for detecting of at least one mutation in at lest of BRCA1 and BRCA2 genes in a mammalian test subject. This particular kit of the invention comprises: (a) means for obtaining a sample of said subject; (b) at least one detecting molecule or a collection of at least two detecting molecules specific for determination of the expression of at least one marker gene or a collection of at least two marker genes. According to a particular embodiment, these marker genes may be selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; AUH, AU RNA binding protein/enoyl-Coenzyme A hydratase; MID1IP1, MID1 interacting protein 1 (gastrulation specific G12 homolog (zebrafish)); RGS16, regulator of G-protein signaling 16; MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; DNAJC12, DnaJ (Hsp40) homolog, subfamily C, member 12; IFI44L, interferon-induced protein 44-like; SARS, seryl-tRNA synthetase; SMURF2, SMAD specific E3 ubiquitin protein ligase 2; SFRS18 (C6ORF111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D, as set forth in Table 4. (c) at least one detecting molecule or a collection of at least two detecting molecules specific for determination of the expression of at least one control reference gene or a collection of at least two control reference genes. According to a specific embodiment, these control reference genes may be selected from the group consisting of: RPS9, HSPCB, Eukaryotic 18S-rRNA and β-actin. The kit of the invention may optionally further comprise (d) optionally, at least one control sample, that may be at least one of a negative control sample and a positive control sample. Alternatively or additionally, the kit of the invention may comprise a standard curve/s illustrating the expression of the marker genes and optionally of the control genes in a control sample.

The kit of the invention may further comprise (e) instructions for carrying out the detection and quantification of expression of the marker genes and of the control reference gene in the tested sample;

Still further, the kit of the invention comprises (f) instructions for evaluating the differential expression of the marker gene in the tested sample and of a control reference gene in the sample as compared with the expression of the marker gene and control reference gene in the control sample, or as compared with a predetermined value indicating and distinguishing between the carrier and the non-carrier populations.

According to one embodiment, the negative control may be obtained from a non-carrier subject and a positive control may be obtained from a subject which is a carrier of at least one mutation in at least one of BRCA1 and BRCA2 genes.

According to one embodiment, the detecting molecules comprised within the kits of the invention may be isolated nucleic acid molecules or isolated amino acid molecules, or any combination thereof.

According to one specific and preferred embodiment, the detecting molecules comprised within the kits of the invention may be isolated nucleic acid molecules. Such molecules may be preferably, isolated oligonucleotides, each oligonucleotide specifically hybridizes to a nucleic acid sequence of the RNA products of at least one of the marker gene of the invention, as set forth in Table 4.

Accordingly, the kit of the invention may therefore comprise as the detecting molecule for the control reference genes, oligonucleotides which specifically hybridize to a nucleic acid sequence of the RNA products of at least one control reference gene selected from the group consisting of: RPS9, HSPCB, Eukaryotic 18S-rRNA and β-actin.

According to a preferred embodiment, such oligonucleotide may be a pair of primers or nucleotide probe or any combination, mixture or collection thereof.

According to such specific and particular embodiment, the primers and probes used by the kit of the invention may be derived from regions of the genes that are also defined as amplicons (selected regions for amplification). Examples for amplicons used are demonstrated by Table 4, which also discloses partial sequences of the amplicons (SEQ ID NO. 25 to 48) used in the following Examples. It should be appreciated that primers and probes may be derived from any other amplicon in the listed marker genes described by the invention.

In another embodiment, the present invention relates in part to kits comprising sufficient materials for performing one or more of the diagnostic methods described by the invention. In preferred embodiments, a kit includes one or more materials selected from the following group in an amount sufficient to perform at least one assay.

Thus, according to another optional embodiment, the kit of the invention may further comprise at least one reagent for performing a nucleic acid amplification based assay. Such nucleic acid amplification assay may be any one of Real Time PCR, micro arrays, PCR, in situ Hybridization and Comparative Genomic Hybridization.

Control nucleic acid members may be present on the array including nucleic acid members comprising oligonucleotides or nucleic acids corresponding to genomic DNA, housekeeping genes, vector sequences, plant nucleic acid sequence, negative and positive control genes, and the like. Control nucleic acid members are calibrating or control genes whose function is not to tell whether a particular “key” marker gene of interest is expressed, but rather to provide other useful information, such as background or basal level of expression. Therefore, it should be appreciated that the measured expression levels for each marker gene is being normalized with the expression levels of a reference control gene.

Preferred control samples may be selected from HSPCB, RPS9, Eukaryotic 18S-rRNA and β-actin. Optionally, other control nucleic acids may be spotted on the array and used as target expression control nucleic acids.

According to an alternative embodiment, the detecting molecule comprised within the kits of the invention may be an isolated amino acid molecule, for example, isolated polypeptides, each polypeptide binds selectively to the protein product of at least one of the marker genes of the invention, as set forth in Table 4. Alternatively, the detecting polypeptides provided by the kits of the invention bind selectively o at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4, at least six, at least seven, at least eight, at least nine, at least ten, at least eleven, at least twelve, at least thirteen, at least fourteen, at least fifteen, at least sixteen, at least seventeen or at least eighteen of the eighteen marker genes listed in Table 7, or at least six, at least seven, at least eight, at least nine, at least ten, at least eleven, at least twelve or at least thirteen, of the thirteen marker genes listed in Table 8.

Accordingly, the detecting molecules specific for the control reference genes may be isolated polypeptides. Each polypeptide binds selectively to the protein product of at least one control reference gene selected from the group consisting of RPS9, HSPCB, Eukaryotic 18S-rRNA and β-actin.

According to a specific embodiment where the detecting molecule is an isolated antibody the kit of the invention may further comprise at least one reagent for performing an immuno assay, such as ELISA, a RIA, a slot blot, a dot blot, immunohistochemical assay, FACS, a radio-imaging assay, Western blot or any combination thereof.

According to another embodiment, the kits provided by the invention may further comprise suitable means and reagents for preparing or isolating at least one of nucleic acids and amino acids from said sample.

The invention further provides specific kits for the detection of at least one mutation of BRCA1 gene in a biological sample of a subject, according to a preferred embodiment, such kit may comprises detection molecule specific for a marker gene or a collection of at least two marker genes. These specific genes exhibiting a differential expression in BRCA1 carriers may be selected from the group consisting of: AUH, AU RNA binding protein/enoyl-Coenzyme A hydratase; RGS16, regulator of G-protein signaling 16; MARCH7, membrane-associated ring finger (C3HC4) 7; DNAJC12, DnaJ (Hsp40) homolog, subfamily C, member 12; IFI44L, interferon-induced protein 44-like; SARS, seryl-tRNA synthetase; and SMURF2, SMAD specific E3 ubiquitin protein ligase 2.

It should be further appreciated that in case of detection of BRCA1 mutation, the marker gene may be selected from genes demonstrated by the invention as exhibiting differential expression of about 1.5 folds. Such genes may be selected from any of the genes set forth in Table 5.

Still further, the invention also provides a specific kit for the detection of at least one mutation of BRCA2 gene in a biological sample of a subject. According to a preferred embodiment, such kit may comprises detection molecule specific for a marker gene or a collection of at least two marker genes. These specific genes exhibiting a differential expression in BRCA2 carriers may be selected from the group consisting of: RAB3GAP1, RAB3 GTPase activating protein subunit 1 (catalytic); NFAT5, nuclear factor of activated T-cells 5, tonicity-responsive; MRPS6, mitochondrial ribosomal protein S6; MID1IP1, MID1 interacting protein 1 (gastrulation specific G12 homolog (zebrafish)); MARCH7, membrane-associated ring finger (C3HC4) 7; NR3C1, nuclear receptor subfamily 3, group C, member 1 (glucocorticoid receptor); ELF1, E74-like factor 1 (ets domain transcription factor); RPS6KB1, ribosomal protein S6 kinase, 70 kDa, polypeptide 1; STAT5A, signal transducer and activator of transcription 5A; YTHDF3, YTH domain family, member 3; SFRS18 (C6ORF111), splicing factor, arginine/serine-rich 18; NR4A2, nuclear receptor subfamily 4, group A, member 2; CDKN1B, cyclin-dependent kinase inhibitor 1B (p27, Kip1); and EIF3D, eukaryotic translation initiation factor 3, subunit D.

It should be further appreciated that in case of detection of BRCA2 mutations, the marker gene may be selected from genes demonstrated by the invention as exhibiting differential expression of about 2 folds. Such genes may be selected from any of the genes set forth in Table 6.

It should be noted that detection of a mutation in any one of BRCA1 or BRCA2 genes may be an indicative of an increased genetic predisposition of the carrier subject to a cancerous disorder associated with mutations in at least one of BRCA1 and BRCA2. Such cancerous disorder may be any disorder of the group consisting of: breast, ovary, pancreas and prostate carcinomas. Therefore, the kits of the invention may be applicable for the detection and preferably, the early detection of such cancerous disorders, particularly of breast carcinoma and ovarian carcinoma.

More specifically, for nucleic acid microarray kits, the kits may generally comprise probes attached to a support surface. The probes may be labeled with a detectable label. In a specific embodiment, the probes are specific for an exon(s), an intron(s), an exon junction(s), or an exon-intron junction(s)), of RNA products of 1, 2, 3, 4, 5, 6, 7, 8, 9, 10, 11, 12, 13, 14, 15, 16, 17, 18, 19 and 20 or more or any combination of the marker genes of the invention. According to one specific embodiment, the probes provided by the kit of the invention may be specific for an exon(s), an intron(s), an exon junction(s), or an exon-intron junction(s)), of RNA products of at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4, at least six, at least seven, at least eight, at least nine, at least ten, at least eleven, at least twelve, at least thirteen, at least fourteen, at least fifteen, at least sixteen, at least seventeen or at least eighteen of the eighteen marker genes listed in Table 7, at least six, at least seven, at least eight, at least nine, at least ten, at least eleven, at least twelve or at least thirteen, of the thirteen marker genes listed in Table 8, or any combination of the marker genes of the invention. The microarray kits may comprise instructions for performing the assay and methods for interpreting and analyzing the data resulting from the performance of the assay. The kits may also comprise hybridization reagents and/or reagents necessary for detecting a signal produced when a probe hybridizes to a target nucleic acid sequence. Generally, the materials and reagents for the microarray kits are in one or more containers or compartments. Each component of the kit is generally in its own a suitable container.

For Real-Time RT-PCR kits, the kits generally comprise pre-selected primers specific for particular RNA products (e.g., an exon(s), an intron(s), an exon junction(s), and an exon-intron junction(s)) of 1, 2, 3, 4, 5, 6, 7, 8, 9, 10, 11, 12, 13, 14, 15, 16, 17, 18, 19, 20 or all or any combination of the marker genes of the invention. The RT-PCR kits may also comprise enzymes suitable for reverse transcribing and/or amplifying nucleic acids (e.g., polymerases such as Taq), and deoxynucleotides and buffers needed for the reaction mixture for reverse transcription and amplification. The RT-PCR kits may also comprise probes specific for RNA products of 1, 2, 3, 4, 5, 6, 7, 8, 9, 10, 11, 12, 13, 14, 15, 16, 17, 18, 19, 20 or more or all, or any combination of the marker genes of the invention. The probes may or may not be labeled with a detectable label (e.g., a fluorescent label). According to one specific embodiment, the probes or primers provided by the kit of the invention may be specific for an exon(s), an intron(s), an exon junction(s), or an exon-intron junction(s)), of RNA products of at least six, at least seven, at least eight, at least nine, at least ten, at least eleven, at least twelve, at least thirteen, at least fourteen, at least fifteen, at least sixteen, at least seventeen, at least eighteen, at least nineteen or at least twenty of the twenty marker genes listed in Table 4, at least six, at least seven, at least eight, at least nine, at least ten, at least eleven, at least twelve, at least thirteen, at least fourteen, at least fifteen, at least sixteen, at least seventeen or at least eighteen of the eighteen marker genes listed in Table 7, at least six, at least seven, at least eight, at least nine, at least ten, at least eleven, at least twelve or at least thirteen, of the thirteen marker genes listed in Table 8, or any combination of the marker genes of the invention. Each component of the RT-PCR kit is generally in its own suitable container. Thus, these kits generally comprise distinct containers suitable for each individual reagent, enzyme, primer and probe. Further, the RT-PCR kits may comprise instructions for performing the assay and methods for interpreting normalizing and analyzing the data resulting from the performance of the assay.

For antibody based kits, the kit can comprise, for example: (1) a first antibody (which may or may not be attached to a support) which binds to protein of interest (e.g., a protein product of 1, 2, 3, 4, 5, 6, 7, 8, 9, 10, 11, 12, 13, 14, 15, 16, 17, 18, 19 or 20 or any combination of the marker genes of the invention); and, optionally, (2) a second, different antibody which binds to either the protein, or the first antibody and is conjugated to a detectable label (e.g., a fluorescent label, radioactive isotope or enzyme).

The antibody-based kits may also comprise beads for conducting an immunoprecipitation assay.

Each component of the antibody-based kits is generally in its own suitable container. Thus, these kits generally comprise distinct containers suitable for each antibody. Further, the antibody-based kits may comprise instructions for performing the assay and methods for normalizing interpreting and analyzing the data resulting from the performance of the assay.

It should be thus appreciated that any of the kits of the invention may optionally further comprise solid support, such as plates, beads, tube or containers. These may be specifically adopted for performing different detection steps or any nucleic acid amplification based assay or immuno assay, as described for example by the method of the invention. It should be further noted that any substance or ingredient comprised within any of the kits of the invention may be attached, embedded, connected, linked, placed, glued or fused to any solid support.

It should be noted that any of the detecting molecules used by the compositions, methods and kits of the invention may be labeled by a detectable label. The term “detectable label” as used herein refers to a composition or moiety that is detectable by spectroscopic, photochemical, biochemical, immunochemical, electromagnetic, radiochemical, or chemical means such as fluorescence, chemifluoresence, or chemiluminescence, or any other appropriate means. Preferred detectable labels are fluorescent dye molecules, or fluorochromes, such fluorescein, phycoerythrin, CY3, CY5, allophycocyanine, Texas Red, peridenin chlorophyll, cyanine, FAM, JOE, TAMRA, tandem conjugates such as phycoerythrin-CY5, and the like. These examples are not meant to be limiting.

It is to be understood that any polynucleotide or polypeptide or any combination thereof described by the invention may be useful as a marker for a disease, disorder or condition, and such use is to be considered a part of this invention.

It should be appreciated that all methods and kits described herein, preferably comprise any of the compositions of the invention.

It should be recognized that the nucleic acid sequences and/or amino acid sequences used by the kits of the present invention relate, in some embodiments, to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that the terms “oligonucleotide” and “polynucleotide”, or “peptide” and “polypeptide”, may optionally be used interchangeably.

According to a specifically preferred embodiment, the marker genes used by any of the compositions, methods and kits of the invention may be selected from the genes as set forth in any one of Tables 4, 7 and 8.

Table 8 discloses the 13 most statistically significant differentially-expressed genes. Go analysis demonstrated that the genes are related to apoptosis, cell signaling, and cell cycle that may be of importance to cancer pathophysiology. All the selected 13 genes were under-expressed compared to controls.

According to another embodiment, the marker gene may be RAB3 GTPase activating protein subunit 1 (catalytic).

Members of the RAB3 protein family are implicated in regulated exocytosis of neurotransmitters and hormones. RAB3GAP1, which is involved in regulation of RAB3 activity, is a heterodimeric complex consisting of a 130-kD catalytic subunit and a 150-kD noncatalytic subunit. RAB3GAP1 specifically converts active RAB3-GTP to the inactive form RAB3-GDP. NCBI accession number: NM_{—}012233.1, as also denoted by SEQ ID NO. 1. It should be noted that the assay ID of this marker gene (by Applied Biosystems) is Hs00326824_m1.

According to another embodiment, the marker gene may be Nuclear factor of activated T-cells 5, tonicity-responsive.

NFAT5 is an integrin, a receptor for extracellular matrix (ECM) ligands, and a critical regulator of the invasive phenotype. Previous studies using cell lines derived from human breast and colon carcinomas provide evidence that NFAT5 is expressed in invasive human ductal breast carcinomas and participates in promoting carcinoma invasion.

NFAT5 is also involved in cellular proliferation. NFAT5 mRNA expression is particularly high in proliferating cells. Inhibition of NFAT5 in embryonic fibroblasts resulted in cell cycle arrest. Under-expression of NFAT5 mRNA in our study may indicate another effort of BRCA1/2 heterozygous lymphocytes to induce cell cycle arrest in response to an insufficient DNA repair process.

The NFAT5 gene is widely transcribed and encodes a protein of 1,455 aa. In contrast to the conventional NFAT proteins, NFAT1-4, which shows high and moderate sequence identity in their DNA-binding and N-terminal regulatory domains, respectively, NFAT5 exhibits a clear relation to NFAT proteins only in its Rel-like DNA-binding domain. The DNA-binding specificity of NFAT5 is similar to that of NFAT1, but the NFAT5 DNA-binding domain differs from the DNA-binding domains of NFAT1-4 in that it does not cooperate with Fos/Jun at NFAT:AP-1 composite sites. A striking feature of NFAT5 is its constitutive nuclear localization that is not modified on cellular activation. Taken together, the data presented by the present invention indicate that NFAT5 is a target of signaling pathways distinct from those that regulates NFAT1-4, and that it is likely to modulate cellular processes in a wide variety of cells. NCBI accession number: NM_{—}006599, as also denoted by SEQ ID NO. 2. It should be noted that the assay ID of this marker gene (by Applied Biosystems) is Hs00232437_m1.

According to another embodiment, the marker gene may be the nuclear encoded Mitochondrial ribosomal protein S6 (MRPS6), a building block of the human mitoribosome of the oxidative phosphorylation system (OXPHOS). Impairments in mitochondrial OXPHOS have been linked to the pathogenesis of tumor development. The mtDNA encoded OXPHOS genes play a key role in transformation of breast epithelial cells. It was reported that down-regulation of claudin-1 and -7 leads to neoplastic transformation of breast epithelial cells, and claudin-1 and -7 were also down-regulated in primary breast tumors. Multiple pathways involved in mitochondria-to-nucleus retrograde regulation contribute to transformation of breast epithelial cells.

The expression of a gene encoding the mitochondria ribosomal protein S6 (MRPS6) had the highest combined mean fold change and topped the list of regulated genes.

Multiregional gene expression profiling identifies MRPS6 as a possible candidate gene for Parkinson's disease. NCBI accession number: NM_{—}032476.2, as also denoted by SEQ ID NO. 3. It should be noted that the assay ID of this marker gene (by Applied Biosystems) is Hs00606808_m1.

According to another embodiment, the marker gene may be AU RNA binding protein/enoyl-Coenzyme A hydratase. AUH gene encodes an RNA-binding protein with intrinsic enzymatic activity. It was suggested, that its hydratase and AU-binding functions are located on different domains within a single polypeptide.

It was shown that 3-methylglutaconyl-CoA hydratase, a key enzyme of leucine degradation, is encoded by the AUH gene. NCBI accession number: NM_{—}001698, as also denoted by SEQ ID NO. 4. It should be noted that the assay ID of this marker gene (by Applied Biosystems) is H s00156044_m1.

According to another embodiment, the marker gene may be MID1 interacting protein 1 (gastrulation specific G12-like (zebrafish).

MID1 is a gene which encodes a TRIM/RBCC protein that is anchored to the microtubules. The association of Mid1 with the cytoskeleton is regulated by dynamic phosphorylation, through the interaction with the alpha4 subunit of phosphatase 2A (PP2A). Mid1 acts as an E3 ubiquitin ligase, regulating PP2A degradation on microtubules.

NCBI accession number: NM_{—}021242, as also denoted by SEQ ID NO. 5. It should be noted that the assay ID of this marker gene (by Applied Biosystems) is Hs00221999_m1.

According to another embodiment, the marker gene may be Regulator of G-protein signaling 16. Members of the ‘regulator of G protein signaling’ (RGS) gene family encode proteins that stimulate the GTPase activities of G protein alpha-subunits. RGS16 is widely expressed as an approximately 2.4-kb mRNA and that its expression is induced by mitogenic signals. Over-expression of RGS16 inhibits G protein-coupled mitogenic signal transduction and activation of the mitogen-activated protein kinase (MAPK) signaling cascade. NCBI accession number: NM_{—}002928.3, as also denoted by SEQ ID NO. 6. It should be noted that the assay ID of this marker gene (by Applied Biosystems) is Hs00161399_m1.

According to another embodiment, the marker gene may be Membrane-associated ring finger (C3HC4) 7. The MARCH-family of proteins regulates endocytosis of cell surface receptors (e.g., transferrin receptor, histocompatibility antigens and Fas; type I as well as type II transmembrane domains) via ubiquitination. A RING finger consists of a double ring structure containing 8 metal binding cysteine and histidine residues that coordinate two zinc ions. RING fingers of E3 ligases can be formed by different configurations of histidine and cysteine residues. The most frequently found ‘classical’ C3HC4 RING domains are involved in many different cellular events. Examples are c-CBL, which functions in ubiquitin-dependent lysosomal trafficking and BRCA1, which affects cell cycle progression through its ligase activity via a mechanism that is still elusive. RING fingers with a C3H2C3 configuration are found in membrane associated E3 ligases catalyzing ubiquitination of degradation substrates occurring in the secretory pathway, especially the ER, and endolysosomal compartments. NCBI accession number: NM_{—}022826.2, as also denoted by SEQ ID NO. 7. It should be noted that the assay ID of this marker gene (by Applied Biosystems) is Hs00224521_m1.

According to another embodiment, the marker gene may be Nuclear receptor subfamily 3 (glucocorticoid receptor) (NR3C1). Of the 2 isoforms of the glucocorticoid receptor generated by alternative splicing, GR-alpha is a ligand-activated transcription factor that, in the hormone-bound state, modulates the expression of glucocorticoid-responsive genes by binding to a specific glucocorticoid response element (GRE) DNA sequence. In contrast, GR-beta does not bind glucocorticoids and is transcriptionally inactive. It was demonstrated that GR-beta is able to inhibit the effects of hormone-activated GR-alpha on a glucocorticoid-responsive reporter gene in a concentration-dependent manner. The inhibitory effect appeared to be due to competition for GRE target sites. Since RT-PCR analysis showed expression of GR-beta mRNA in multiple human tissues, GR-beta may be a physiologically and pathophysiologically relevant endogenous inhibitor of glucocorticoid action and may participate in defining the sensitivity of tissues to glucocorticoids. NCBI accession number: X03348, as also denoted by SEQ ID NO. 8. It should be noted that the assay ID of this marker gene (by Applied Biosystems) is Hs00353740_m1

According to another embodiment, the marker gene may be E74-like factor 1 (ets domain transcription factor). E74-like factor (1 ELF1) is a lymphoid-specific ETS transcription factor that regulates inducible gene expression during T cell activation and is known to be a key component in the transcriptional program during hematopoietic stem cell development. It has been demonstrated that ELF1 contains a sequence motif that is highly related to the RB (retinoblastoma) binding sites of several viral oncoproteins and binds to the pocket region of RB both in vitro and in vivo. Other results demonstrated that RB interacts specifically with this lineage-restricted ETS transcription factor. The interaction may be important for the coordination of lineage-specific effector function. A comparative study of mouse and human breast cancer SAGE data revealed a very significant down-regulation in expression of transcription factor ELF1 in mouse and human breast carcinoma tumors. The decreased expression of ELF1 observed in breast cancer appears contrary to most Ets transcription factors, where over-expression is usually associated with malignant processes. In a recent in vivo study, E74-like factor-1 (Elf-1) was found as a promoter binding factor of human Pygopus2 gene that is over-expressed in a high proportion of breast and epithelial ovarian malignant tumors, and is required for the growth of several cell lines derived from these carcinomas. The control of hPygo2 expression via Elf-1 may be regulated coordinately with the cell cycle via auto-activation of Wnt-dependent signaling components in cancer. The under-expression of ELF1 in BRCA1/2 heterozygous lymphocytes due to irradiation may be a deviation from the normal process of Wnt-dependent signaling during cell cycle. NCBI accession number: NM_{—}172373, as also denoted by SEQ ID NO. 9. It should be noted that the assay ID of this marker gene (by Applied Biosystems) is Hs00152844_m1.

According to another embodiment, the marker gene may be similar to ribosomal protein S6 kinase, polypeptide 1 (RPS6KB1).

RPS6KB1 mediates the rapid phosphorylation of ribosomal protein S6 on multiple serine residues in response to insulin or several classes of mitogens. Acquisition of S6 protein kinase catalytic function is restricted to the most extensively phosphorylated polypeptides. In mammals, mammalian target of rapamycin cooperates with PI3K-dependent effectors in a biochemical signaling pathway to regulate the size of proliferating cells. NCBI accession number: NM_{—}003161, as also denoted by SEQ ID NO. 10. It should be noted that the assay ID of this marker gene (by Applied Biosystems) is Hs00177357_m1.

According to another embodiment, the marker gene may be Signal transducer and activator of transcription 5A. STATs, such as STAT5, are proteins that serve the dual function of signal transducers and activators of transcription in cells exposed to signaling polypeptides. More than 30 different polypeptides cause STAT activation in various mammalian cells. STAT5 was identified as the protein most notably induced in response to T-cell activation with IL2. They hypothesized that STAT5 may govern the effects of IL2 during the immune response. NCBI accession number: NM_{—}003152, as also denoted by SEQ ID NO. 11. It should be noted that the assay ID of this marker gene (by Applied Biosystems) is Hs00559643_m1.

According to another embodiment, the marker gene may be YTH domain family, member 3 [Mehrle, A, et al. Nucleic Acids Res. 1; 34 (Database issue):D415-8. Related Articles, Links (2006)]. NCBI accession number: NM_{—}152758.4, as also denoted by SEQ ID NO. 12. It should be noted that the assay ID of this marker gene (by Applied Biosystems) is

More particularly, according to one embodiment, such marker gene may be DnaJ (Hsp40) homolog, subfamily C, member 12. DnaJ/HSP40 proteins, which are molecular chaperones of HSP70 proteins, contain all or a combination of 4 domains: an N-terminal J domain; a glycine/phenylalanine (G/F)-rich domain; a central repeat region (CRR), and a weakly conserved C-terminal domain. The J domain, which is believed to mediate interaction with HSP70 proteins, contains a highly conserved histidine-proline-aspartate (HPD) tripeptide. J domain-only proteins are members of a subclass of the HSP40/DnaJ family that possess the J domain as well as a highly conserved C terminus, but lack the G/F-rich and CRR domain. NCBI accession number NM_{—}021800, as also denoted by SEQ ID NO. 13. It should be noted that the assay ID of this marker gene (by Applied Biosystems) is Hs00222318_m1.

According to another embodiment, the marker gene may be Interferon-induced protein 44-like (IFI44L). The biological function of this gene is unknown. [Suzuki, Y. et al., Gene. 24:200(1-2):149-56 (1997)]. NCBI accession number: NM_{—}006820, as also denoted by SEQ ID NO. 14. It should be noted that the assay ID of this marker gene (by Applied Biosystems) is Hs00199115_m1.

According to another embodiment, the marker gene may be Seryl-tRNA synthetase. The human seryl-tRNA synthetase has been expressed in E. coli, purified (95% pure as determined by SDS/PAGE). The human seryl-tRNA synthetase sequence (514 amino acid residues) shows significant sequence identity with seryl-tRNA synthetases from E. coli (25%), Saccharomyces cerevisiae (40%), Arabidopsis thaliana (41%) and Caenorhabditis elegans (60%). The functional studies show that the enzyme aminoacylates calf liver tRNA and prokaryotic E. coli tRNA. NCBI accession number: NM_{—}006513, as also denoted by SEQ ID NO. 15. It should be noted that the assay ID of this marker gene (by Applied Biosystems) is Hs00197856_m1.

According to another embodiment, the marker gene may be SMAD specific E3 ubiquitin protein ligase 2. Ubiquitin-mediated proteolysis regulates the activity of diverse receptor systems. SMAD specific E3 ubiquitin protein ligase 2 (SMURF2) associated constitutively with SMAD7. Western blot analysis showed that SMURF2 selectively regulated the expression of SMAD2 and, to some extent, SMAD1, but not SMAD3, through an ubiquitination- and proteasome-dependent degradation process catalyzed by the HECT ligase.

It was found that telomere attrition in human fibroblasts induced SMURF2 upregulation, and this upregulation was sufficient to produce the senescence phenotype. Infection of early passage fibroblasts with retrovirus carrying SMURF2 led to morphologic and biochemical alterations characteristic of senescence, including altered gene expression and reversal of cellular immortalization by TERT. It was further showed that SMURF2 activated senescence through the RB (180200) and p53 pathways. NCBI accession number: NM_{—}022739.3, as also denoted by SEQ ID NO. 16. It should be noted that the assay ID of this marker gene (by Applied Biosystems) is Hs00224203_m1.

In yet another embodiment, the marker gene may be splicing factor, arginine/serine-rich 18 (SFRS18 (C6ORF111)). This gene has an undefined function. NCBI accession number: NM_{—}032870.2, as also denoted by SEQ ID NO. 17. It should be noted that the assay ID of this marker gene (by Applied Biosystems) is Hs00369090_m1.

According to another embodiment, the marker gene may be Nuclear receptor subfamily 4, group A, member 2. Nuclear receptor subfamily 4, group A, member 2, is a gene encoding a member of the steroid/thyroid hormone family of receptors. The receptor, called NOT (nuclear receptor of T cells) by them, has all of the structural features of steroid/thyroid hormone receptors but is rapidly and only very transiently expressed after cell activation. NURR1 and PITX3 cooperatively promoted terminal maturation of murine and human embryonic stem cell. NCBI accession number: NM_{—}006186, as also denoted by SEQ ID NO. 18. It should be noted that the assay ID of this marker gene (by Applied Biosystems) is Hs00428691_m1.

According to another embodiment, the marker gene may be Cyclin-dependent kinase inhibitor 1B-CDKN1B (p27, Kip1).

Cyclin-dependent kinase (CDK) activation requires association with cyclins (e.g., CCNE1) and phosphorylation by CAK (CCNH), and leads to cell proliferation. Inhibition of cellular proliferation occurs upon association of CDK inhibitor (e.g., CDKN1B) with a cyclin-CDK complex. It was showed that expression of CCNE1-CDK2 at physiologic levels of ATP results in phosphorylation of CDKN1B at thr187, leading to elimination of CDKN1B from the cell and progression of the cell cycle from G1 to S phase. At low ATP levels, the inhibitory functions of CDKN1B are enhanced, thereby arresting cell proliferation. The CDKN1B gene encodes the p27(kip1) protein that functions as an inhibitor of cyclin dependent kinase-2, and shows loss of expression in a large percentage of BRCA1 and BRCA2 breast cancer cases. Additionally, CDKN1B is a suspected genetic modifier that may explain differences in the estimated risk that is found to be higher in studies based on multiple case families than in population-based studies. Immuno-detection of p27 has been used as a prognostic factor in a variety of cancer types, with low expression levels being correlated with reduced median survival time. Furthermore, characterization of p27-deficient breast cancer cell lines which promoted progression in mouse tumor migration experiments have provided evidence that p27 plays an essential role in the restriction of breast cancer progression.

NCBI accession number: BC001971, as also denoted by SEQ ID NO. 19. It should be noted that the assay ID of this marker gene (by Applied Biosystems) is

According to another embodiment, the marker reference gene may be Eukaryotic translation initiation factor 3, subunit 7 zeta, 66/67 kDa.

Eukaryotic initiation factor-3 (eIF3), the largest of the eIFs, is a multiprotein complex of approximately 600 kD that binds to the 40S ribosome and helps maintain the 40S and 60S ribosomal subunits in a dissociated state. It is also thought to play a role in the formation of the 40S initiation complex by interacting with the ternary complex of eIF2/GTP/methionyl-tRNA, and by promoting mRNA binding. NCBI accession number: NM_{—}003753, as also denoted by SEQ ID NO. 20. It should be noted that the assay ID of this marker gene (by Applied Biosystems) is Hs00388727_m1.

According to one embodiment, the control reference gene may be Eukaryotic 18S rRNA. NCBI accession number: X03205.1, as also denoted by SEQ ID NO. 21. It should be noted that the assay ID of this marker gene (by Applied Biosystems) is Hs99999901_s1.

According to another embodiment, the control reference gene may be ribosomal protein S9. NCBI accession number: NM_{—}001013.3, as also denoted by SEQ ID NO. 22. It should be noted that the assay ID of this marker gene (by Applied Biosystems) is Hs02339426_g1.

According to another embodiment, the control reference gene may be Actin, beta. Interaction of phospholipase D with actin microfilaments regulates cell proliferation, vesicle trafficking, and secretion. Localization of beta-actin mRNA to sites of active actin polymerization modulates cell migration during embryogenesis, differentiation, and possibly carcinogenesis. In immunoprecipitation studies of embryonic fibroblasts from wild type and knockout mice deficient in the arginylation enzyme Atel (607103), Karakozova et al. [Karakozova M. et al. Science 313 (5784): 192-6 (2006)] found that approximately 40% of intracellular beta-actin is arginylated in vivo. Karakozova et al. (2006) found that arginylation of beta-actin regulates cell motility. Mammalian cytoplasmic actins are the products of 2 different genes and differ by many amino acids from muscle actin. NCBI accession number: NM_{—}001101, as also denoted by SEQ ID NO. 23. It should be noted that the assay ID of this marker gene (by Applied Biosystems) is Hs99999903_m1.

According to another embodiment, the control reference gene may be heat shock protein 90 kDa alpha (cytosolic), class B member 1.

NCBI accession number: NM_{—}007355.2, as also denoted by SEQ ID NO. 24. It should be noted that the assay ID of this marker gene (by Applied Biosystems) is Hs00607336_gH.

According to another embodiment, the marker gene may be Sorting nexin 2. The sorting nexins constitute a large conserved family of hydrophilic molecules that interact with a variety of receptor types. These molecules contain an approximately 100-amino acid region termed the phox homology (PX) domain. NCBI accession number: AF043453.

According to another embodiment, the marker gene may be Hypothetical protein MGC4504. MGC4504 is a homolog protein of ChaC, cation transport regulator homolog 1 (E. coli) CHAC1. CHAC1 molecular function is regulation of cellular ion concentrations which is necessary to sustain a multitude of physiological processes including pH balance and ion homeostasis. NCBI accession number: NM_{—}024111, PubMed ID: 12460671.

According to another embodiment, the marker gene may be Granulysin. Cytolytic T lymphocytes (CTLs) are required for protective immunity against intracellular pathogens. CTLs that kill infected cells through the granule-exocytosis pathway may release 1 or more effector molecules with the capacity to kill the intracellular microbial pathogen directly showed that granulysin is a critical effector molecule of the antimicrobial activity of CTLs. Granulysin is a protein present in cytotoxic granules of CTLs and natural killer (NK) cells. Amino acid sequence comparison indicated that granulysin is a member of the saposin-like protein (SAPLIP) family. Granulysin is located in the cytotoxic granules of T cells, which are released upon antigen stimulation. NCBI accession number: NM_{—}006433.

According to another embodiment, the marker gene may be Serine hydroxymethyltransferase 2 (mitochondrial). The enzyme serine hydroxymethyltransferase (SHMT is a pyridoxal phosphate-dependent enzyme that catalyzes the reversible interconversion of serine and H4PteGlu to glycine and 5, 10-CH2-H4PteGlu with generating of one-carbon units. SHMT is present in both the mitochondria (mSHMT) and the cytoplasm (cSHMT) in mammalian cells. The human SHMT cDNAs encoding the two isozymes have been isolated and the genes localized to chromosomes 12q13 and 17p11.2, respectively. Currently, the metabolic role of the individual SHMT isozymes is not clearly understood. The central role of SHMT isozymes in producing one-carbon-substituted folate cofactors has suggested that the regulation of these enzymes may influence cell growth and proliferation and that they may be targets for the development of antineoplastic agents. NCBI accession number: NM_{—}005412.

According to another embodiment, the marker gene may be Annexin A2. Annexin II, a major cellular substrate of the tyrosine kinase encoded by the SRC oncogene belongs to the annexin family of Ca(2+)-dependent phospholipid- and membrane-binding proteins. By screening a cDNA expression library generated from highly purified human osteoclast-like multinuclear cells (MNC) formed in long-term bone marrow cultures, a candidate clone that stimulated MNC formation was identified. Sequence analysis showed that this cDNA encoded annexin II. Further studies yielded results suggesting that ANX2 is an autocrine factor that enhances osteoclast formation and bone resumption, a previously unknown function for this molecule. NCBI accession number: BC001388.

According to another embodiment, the marker gene may be BTB and CNC homology, basic leucine zipper transcription factor 2. Members of the small Maf family are basic region leucine zipper (bZIP) proteins that can function as transcriptional activators or repressors. Mouse cDNAs encoding Bach1 (602751) and Bach2 were previously identified. Both Bach proteins contain a BTB (broad complex-tramtrack-bric-a-brac) or POZ (poxvirus and zinc finger) protein-interaction domain and a CNC (Cap′n′collar)-type bZIP domain.

Bach1 and Bach2 functioned as transcription repressors in transfection assays using fibroblast cells, but they functioned as a transcriptional activator and repressor, respectively, in cultured erythroid cells. Gel shift analysis showed that when overexpressed, BACH2 binds to MAF recognition elements (MARE). Over expression also resulted in a loss of clonogenic activity. BACH2/CA-1 microsatellite analysis indicated that loss of heterozygosity occurred in 5 of 25 non-Hodgkin lymphoma patients. NCBI accession number: NM_{—}021813.

According to another embodiment, the marker gene may be E2F transcription factor 2. The ability of Myc to induce S phase and apoptosis requires distinct E2F activities. Hence, the induction of specific E2F activities is an essential component in the MYC pathways that control cell proliferation and cell fate decisions. The retinoblastoma tumor suppressor (Rb) pathway is believed to have a critical role in the control of cellular proliferation by regulating E2F activities. E2F1, E2F2, and E2F3 belong to a subclass of E2F factors thought to act as transcriptional activators important for progression through the G1/S transition. NCBI accession number: NM_{—}004091.

According to another embodiment, the marker gene may be Major histocompatibility complex, class II, DQ beta 1. The genes for the heteromeric major histocompatibility complex class II proteins, the alpha and beta subunits, are clustered in the 6p21.3 region. It was suggested that the structure of the DQ molecule, in particular residue 57 of the beta-chain, specifies the autoimmune response against insulin-producing islet cells that leads to insulin-dependent diabetes mellitus. The extremely high polymorphism of HLA class II transmembrane heterodimers is due to a few hypervariable segments present in the most external domain of their alpha and beta chains. Some changes in amino acid sequence are critical in disease susceptibility associations as well as the ability to present processed antigens to T cells. In addition to insulin-dependent diabetes mellitus, an increased frequency of specific alleles at the DQB1 locus has been claimed for narcolepsy, pemphigus vulgaris, and ocular cicatricial pemphigoid. It was found that HLA-DQB1 genotypes encoding aspartate-57 are associated with 3-beta-hydroxysteroid dehydrogenase autoimmunity in Premature ovarian failure. NCBI accession number: NM_{—}002123.

According to another embodiment, the marker gene may be Tensin 3.

Tensin 3 is a cytoplasmic phosphoprotein that localized to integrin-mediated focal adhesions. It binds to actin filaments and contains a phosphotyrosine-binding (PTB) domain, which interacts with the cytoplasmic tails of integrin. In addition, tensin has a Src Homology 2 (SH2) domain capable of interacting with tyrosine-phosphorylated proteins. Furthermore, several factors induce tyrosine phosphorylationof tensin. Thus, tensin functions as a platform for dis/assembly of signaling complexes at focal adhesions by recruiting tyrosine-phosphorylated signaling molecules through the SH2 domain, and also by providing interaction sites for other SH2-containing proteins. An elevated expression of tensin 3 was demonstrated during tumor angiogenesis, so it serves as tumor endothelial marker (TEM). NCBI accession number: NM_{—}022748.

According to another embodiment, the marker gene may be Lysosomal-associated membrane protein 2. The lysosomal membrane plays a vital role in the function of lysosomes by sequestering numerous acid hydrolases that are responsible for the degradation of foreign materials and for specialized autolytic functions. LAMP2 is glycoprotein that constitutes a significant fraction of the total lysosomal membrane glycoproteins. It consists of polypeptides of about 40 kD, with 16 to 20 N-linked saccharides attached to the core polypeptides. LAMP2 is thought to protect the lysosomal membrane from proteolytic enzymes within lysosomes and to act as a receptor for proteins to be imported into lysosomes. NCBI accession number: NM_{—}013995.

According to another embodiment, the marker gene may be Retinoblastoma-like 2 (p130). Retinoblastoma-like 2 is transcription factor, which shown to related to DNA-dependent cell cycle regulation and to negative regulation of progression through cell cycle. RBL2 is essential for telomere length control in human fibroblasts, with loss of either protein leading to longer telomeres. It was proposed that RBL2 forms a complex with RAD50 through RINT1 to block telomerase-independent telomere lengthening. NCBI accession number: NM_{—}005611.

According to another embodiment, the marker gene may be Interleukin 15 receptor, alpha. Interleukin-2 (IL2) and interleukin-15 (IL15) are cytokines with overlapping but distinct biologic effects. Their receptors share 2 subunits, the IL2R beta and gamma chains, which are essential for signal transduction. The IL2 receptor requires an additional IL2-specific alpha subunit (IL2RA) for high-affinity IL2 binding. Confocal microscopy demonstrated that full-length IL15RA was associated primarily with the nuclear membrane, with part of the receptor having an intranuclear localization. It was shown that the IL15/IL15RA complex has enhanced effects on T-cell survival compared with IL15 alone. NCBI accession number: NM_{—}002189.

According to another embodiment, the marker gene may be Cyclin H.

The cdk-activating kinase (CAK) is a multi-subunit protein which phosphorylates and thus activates certain cyclin-dependent protein kinases in the regulation of cell cycle progression. Presence of the CAK complex as a distinct component of TFIIH, suggesting a link between TFIIH (by the phosphorylation of CDC2 or CDK2) and the processes of transcription, DNA repair, and cell cycle progression.

Phosphorylation of mammalian cyclin H by CDK 8 represses both the ability of TFIIH to activate transcription and its C-terminal kinase activity. In addition, mimicking CDK8 phosphorylation of cyclin H in vivo has a dominant-negative effect on cell growth. NCBI accession number: NM_{—}001239.

According to another embodiment, the marker gene may be Stromal antigen 2. A multi-subunit complex, termed cohesin, is likely to be a central player in sister chromatid cohesion. STAD is mammalian analog of Smc1p, Smc3p, and Scc1p. Smc1p and Smc3p belong to a large family of chromosomal ATPases (the structural maintenance of chromosomes [SMC] 1 family), members of which are involved in many aspects of higher order chromosome architecture and dynamics.

NCBI accession number: BC001765.

According to another embodiment, the marker gene may be Ring finger protein 11.

The RING finger is a C3HC4-type zinc finger motif, and members of RING finger proteins are mostly nuclear proteins and the motif is involved in both protein-DNA and protein-protein interactions. Some members of the RING finger family have been implicated in carcinogenesis and cell transformation. For example, a RING finger protein, BRCA1, is a tumor suppressor in an early onset breast cancer. The approximate corresponding cytogenetic location of the ring finger protein 11gene is on chromosome 1p31-p32 region. This region is frequently involved in deletions and chromosomal translocations observed in T-cell acute lymphoblastic leukemia (T-ALL). NCBI accession number: AB024703.

According to another embodiment, the marker gene may be Cyclin T2.

Cyclin T2 is a part of positive transcription elongation factor b (P-TEFb) which is thought to facilitate the transition from abortive to productive elongation by phosphorylating the C-terminal domain (CTD) of the largest subunit of RNA polymerase II. cDNAs encoding human cyclins T1 and T2 was identified. Immunoprecipitation studies demonstrated that CDK9 is complexed with the cyclins T1 and T2 in HeLa cell nuclear extracts. Approximately 80% of CDK9 is complexed with cyclin T1, 10% with cyclin T2a and 10% with T2b. Each complex is an active P-TEFb molecule that can phosphorylate the CTD of RNA polymerase II and cause the transition from abortive elongation into productive elongation. When expressed in mammalian cells, all 3 CDK9/cyclin T combinations strongly activated a CMV promoter. Northern blot analysis revealed that cyclin T2 was expressed as multiple mRNAs in all human tissues tested. NCBI accession number: NM_{—}001241.

It should be further appreciated that in case of detection of BRCA1 mutation, the marker gene may be selected from genes demonstrated by the invention as exhibiting differential expression of about 1.5 folds. Therefore, according to one embodiment, such genes may be selected from any of the genes set forth in Table 5 (disclosed at the end of the Examples).

In another embodiment, it should recognized that in case of detection of BRCA2 mutations, the marker gene may be selected from genes demonstrated by the invention as exhibiting differential expression of about 2 folds. Therefore, according to another embodiment, such genes may be selected from any of the genes set forth in Table 6 (disclosed at the end of the Examples).

All technical and scientific terms used herein should be understood to have the meaning commonly understood by a person skilled in the art to which this invention belongs, as well as any other specified description.

The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

### Samples Information

Fresh blood samples were obtained from proven unaffected carriers of BRCA1 mutations, 8 unaffected carriers of BRCA2 mutations, and healthy age-matched control women with no individual or family history of cancer.

Individuals heterozygous for BRCA1 and BRCA2 germline mutations were identified from the BRCA1 and BRCA2 predictive testing program in the Institute of Cancer Research Royal Marsden Foundation NHS Trust, Cancer Genetics Carrier Clinic, London, UK and from the Cancer Genetic Clinic of Hadassah University Medical Center, Jerusalem, Israel. Fresh blood samples were collected from unaffected BRCA1/2 heterozygous gene mutation carriers and healthy age-matched control women with no individual or family history of cancer. The mutations in the BRCA1/2 carriers are listed in Table 1. Written informed consent was obtained from all participating individuals prior to inclusion into the study, and the study protocol was approved by the Royal Marsden Locoregional Ethics Committee.

TABLE 1
Characteristics of mutations in BRCA1 and BRCA2 genes in carriers
used for the present invention

| Gene | Mutation |
|---|---|
| BRCA1 | 5382 inc C |
| BRCA1 | Del AG 185 |
| BRCA1 | 185 del AG |
| BRCA1 | 3875 del 4 |
| BRCA1 | Ins C5382 |
| BRCA1 | A > T 1182 |
| BRCA1 | 44184 del |
| BRCA1 | 185 del AG |
| BRCA1 | 3450 del CAAG |
| BRCA2 | 5950 del CT |
| BRCA2 | del TT6503 |
| BRCA2 | C > T9610 |
| BRCA2 | del CA995 |
| BRCA2 | 6503 del TT |
| BRCA2 | 4075del GT |
| BRCA2 | del CA995 |
| BRCA2 | 6174del T |

### Samples Preparation and RNA Extraction

Lymphocytes were collected from blood samples using LymphoPrep kit (Sigma), short-term cultured for 6 days and irradiated with 8 Gray (Gy) at a high dose rate (0.86 Gy/min) using Ortovoltage X-ray machine. One hour later RNA was extracted using Qiagene EZ RNA kit according to manufacturer's instruction for further analysis. The integrity of all RNA samples was verified by 2% agarose gel electrophoresis before use in microarray experiments.

### Microarray Assay

Gene expression profile of the lymphocytes from BRCA1/2 mutation carriers and non-carriers was performed using Affymetrix GeneChip Human Genome U133A 2.0 oligonucleotide arrays. Total RNA from each sample was used to prepare biotinylated target RNA. Briefly, 5 μg was used to generate first-strand cDNA by using a T7-linked oligo(dT) primer. After second-strand synthesis, in vitro transcription was performed with biotinylated UTP and CTP (Affymetrix), resulting in approximately 300-fold amplification of mRNA. The target cDNA generated from each sample was processed as per the manufacturer's recommendation using an Affymetrix GeneChip instrument system. For this reason, spike controls were added to 15 μg of fragmented cRNA before overnight hybridization. Arrays were then washed and stained with streptavidin-phycoerythrin, before being scanned on an Affymetrix GeneChip scanner. A complete description of these procedures is available at: http://www.affvmetrix.com/support/technical/manual/expression_manual.affx. After scanning, array images were assessed by eye to confirm scanner alignment and the absence of significant bubbles or scratches on the chip surface. The 3′/5′ ratios for GAPDH and beta-actin were confirmed to be within acceptable limits and BioB spike controls were found to be present on all chips, with BioC, BioD, and CreX also present in increasing intensity. When scaled to a target intensity of 150 (using Affymetrix MAS 5.0 array analysis software), scaling factors for all arrays were within acceptable limits as were background, Q values and mean intensities.

### Data Analysis

Data analysis was done using GeneSpring GX software (Agilent technologies). Background adjustment, quantile normalization and summarization were done using RMA methodology. The relative expression data for each probe set then generated by normalizes each gene to the median of its own expression intensities across the entire experiment set (per gene normalization). Control probes and genes whose expression does not change across the experiment were removed out from the list before statistical analysis was performed. Differentially expressed genes were analyzed by One-way Welch ANOVA, with p-value cutoff of 0.05 after Benjamini and Hochberg False Discovery Rate multiple testing correction. Average linkage hierarchical clustering of the different experimental samples was obtained for selected genes using Pearson correlation as similarity measure. Bootstrapping analysis was carried out for the assessment of the robustness of the cluster dendogram topology. Cluster members were categorized according to their biological functions using The Database for Annotation, Visualization and Integrated Discovery (DAVID) tools [Dennis G. Jr. et al. Genome Biol. 4(5): 3 (2003)]. Pathway express tool [Khatri P. et al. Nucleic Acids Res. 33(Web Server issue):W762-5 (2005)] was used to characterize the responsive genes on molecular interactions networks in regulatory pathways.

### TaqMan® Quantitative Gene Expression Measurement

To validate the results obtained by the Affymetrix U133 A chips, the inventors have performed TaqMan® verification for expression of 42 selected genes in all experimental samples, using an Applied Biosystems7900 HT Micro Fluidic Card System.

The measurements were performed using an ABI PRISM1 7900HT Sequence Detection Systems described in the products User Guide (http://www.appliedbiosystems.com, CA, USA).

TaqMan® Arrays' 384-wells are pre-loaded with TaqMan® Gene Expression Assays. Each TaqMan® Array evaluates from one to eight cDNA samples generated in a reverse transcription step using random primers on 7900HT Systems. The TaqMan® Array functions as an array of reaction vessels for the PCR step. Relative levels of gene expression are determined from the fluorescence data generated during PCR sing the ABI PRISM®7900HT Sequence Detection System or Applied Biosystems 7900HT Fast Real-Time PCR System Relative Quantitation software. The TaqMan® array technology allows multiple targets to be analyzed per sample with very few pipetting steps, streamlining reaction set-up time, and eliminating the need for liquid handling robotics. TaqMan® arrays provide a standardized format for gene expression studies that permits direct comparison of results across different researchers and laboratories.

cDNA samples for the PCR reaction were prepared by performing reverse transcription of the RNA samples using the High Capacity cDNA Reverse Transcription Kit (Applied Biosystems). For this reaction 2 μg of total RNA in a single 20 A reaction used to obtain up to 10 μg of single stranded cDNA from a single reaction. 100 ng of cDNA samples loaded to plastic tube together with RNase-free water up to total volume of 50 μl and add 50 μl of TaqMan® Universal PCR Master Mix (2×). 100 μl, of the desired sample-specific PCR reaction mix loaded into the fill reservoir on the Place the TaqMan® Array plate. The samples were loaded in triplicates. The TaqMan® Array plates with loaded samples centrifuged at 1200 rpm for 1-2 minutes to ensure complete distribution of the sample specific PCR reaction mix. Following the centrifugation the plate were sealed to isolate the wells of a TaqMan® Array after it is loaded with cDNA samples and master mix. The sealer uses a precision stylus assembly (carriage) to seal the main fluid distribution channels of the array. After sealing procedure, the TaqMan® Arrays were running on the 7900HT instrument.

The extracted delta Ct values (which represent the expression normalized to the ribosomal 18S expression) were grouped according to the resistance pattern of the cell lines. Then, the Student's t-test was performed to compare the expression values in the resistant cell lines to the sensitive cell lines.

### Example 1

### Expression Profile of BRCA1 and BRCA2 Mutations Carriers

In order to identify marker genes potentially applicable for early diagnosis and prognosis of breast cancer patients, and specifically of BRCA1/2 mutation carriers, the inventors analyzed expression profiles of control samples compares to samples of BRCA1/2 mutation carriers under irradiation stress. Therefore, fresh blood samples were obtained from seventeen proven unaffected carriers of BRCA1 mutations, ten unaffected carriers of BRCA2 mutations and twelve healthy age-matched control women with no individual or family history of cancer (were tested negatively for mutations in the BRCA1/2 genes). Lymphocytes prepared from the samples were irradiated, and subsequently RNA extracted from these samples was analyzed using Affimetrix oligonucleotide arrays assay, as described in experimental procedures.

To examine potential relationships between the expression profiles of control and BRCA1/2 mutation carrier samples, microarray analyses were performed. The Affymetrix GeneChip Human Genome U133A 2.0 Array was probed using cDNA obtained from lymphocytes from nine proven unaffected carriers of BRCA1, eight BRCA2 carriers and from ten non-carrier healthy women. For each sample an individual chip was used. Hybridization experiments were carried on RNA extracted from lymphocytes before irradiation and 1 hour following exposure to 8 Gy of ionizing irradiation.

No significant differences in gene expression profiles were detected in a preliminary study using RNA from non-irradiated lymphocytes from the three groups (data not shown). This result is consistent with findings in previous studies [Kote-Jarai Z. et al. Clin. Cancer Res. 12(13):3896-901 (2006); Kote-Jarai Z. et al. Clin. Cancer Res. 10(3):958-63 (2004)]. Following irradiation, differences in gene expression profiles between the three groups were observed.

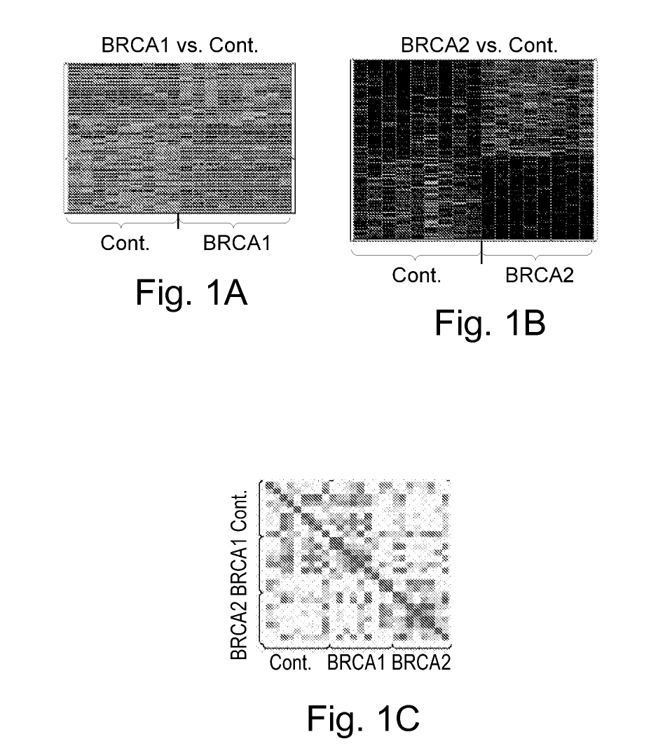

Data was processed used the MAS 5.0 and RMA algorithm to provide a baseline expression level and detection for each probe set. For each probe set, the ratio between expression level of the BRCA1/2 mutation carriers and control samples was calculated. The results of the expression analysis are demonstrated in FIG. 1.

The rule-based clustering method used on the probe sets, demonstrated significantly different expression pattern between either BRCA1 or BRCA2 group as compared to control group (p-value 0.05). For each probe set, the ratio between expression level of the mutation carriers and control samples was calculated. Each probe set was graded as increased, decreased or unchanged. As shown in FIG. 1, clustering of up-regulated genes in BRCA2 mutation carrier group when compared to control group can be clearly observed (FIG. 1) and a sharp distinction in gene expression pattern in BRCA2 mutation carriers is demonstrated. Moreover, expression patterns within a BRCA2 mutation group were highly conserved among all samples (FIG. 1B), whereas gene expression profile of BRCA1 mutation carrier samples is much less homogenous (FIG. 1A).

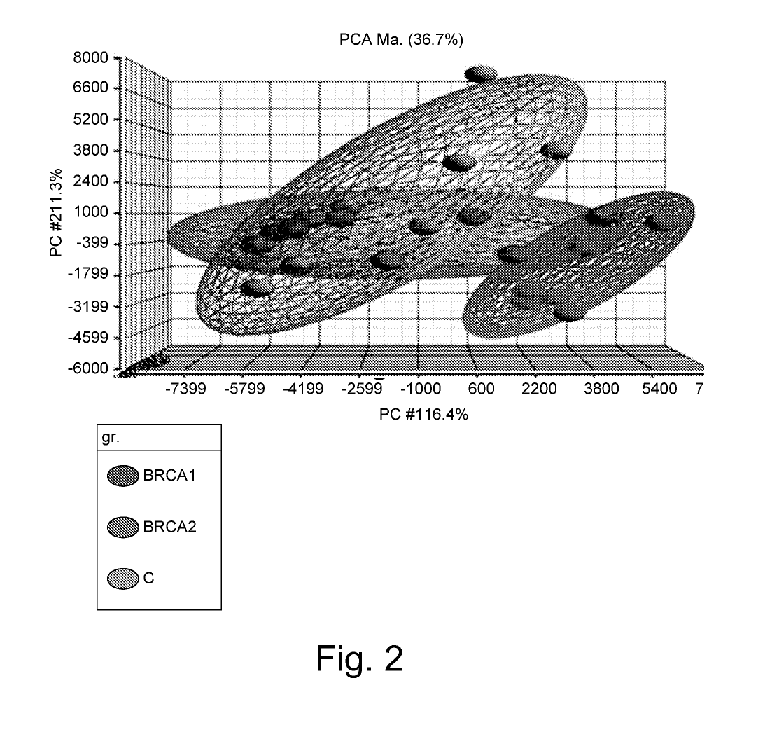

The results of the principal components analysis (PCA) of these genes as demonstrated by FIG. 2, strengthen aforementioned findings. PCA clearly indicate that samples from BRCA2 mutation carriers are well assembled together and almost completely separated from either control or BRCA1 groups. By contrast, BRCA1 group represents more variable expression patterns.

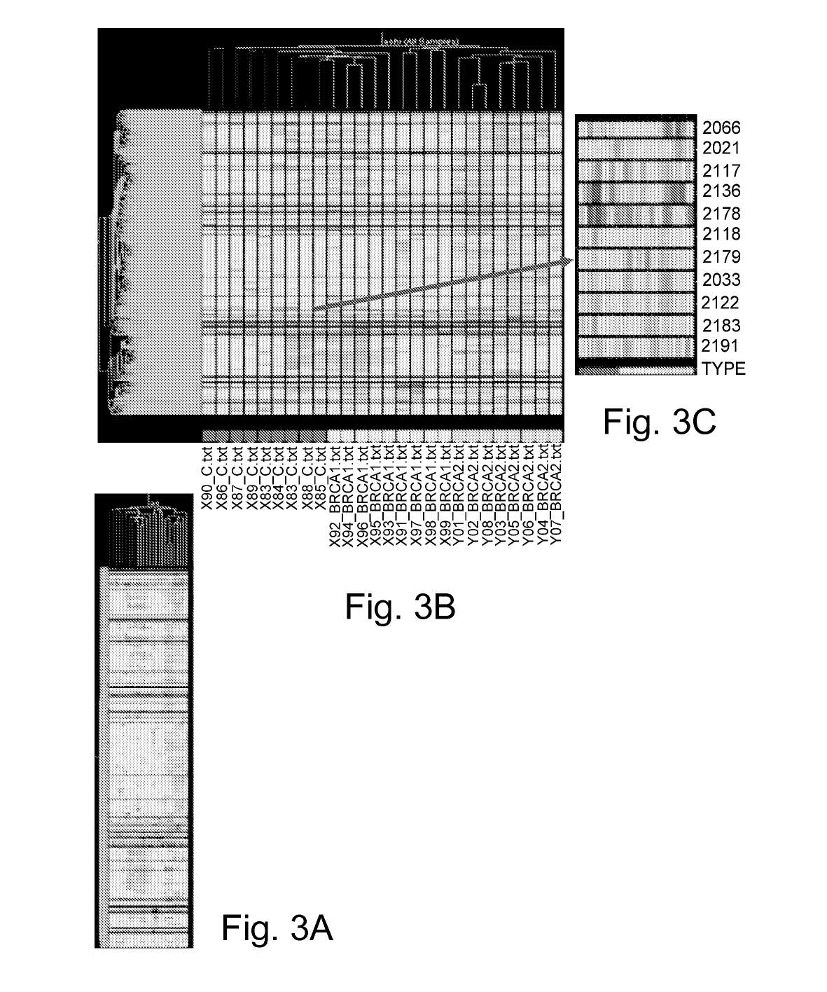

Founder mutations 185delAG, and 5382insC in the BRCA1 gene and 6174delT in the BRCA2 gene are common among Jewish Ashkenazi population (˜0.5%) [Simard J. et al. Natural Genetics 8:392-398 (1994); Takahashi H. et al. Cancer Research 55:2998-3002 (1995); Tonin P. et al. American Journal of Human Genetics 57:189-189 (1995)]. In the general Ashkenazi population, the carrier frequencies of these mutations were estimated to be −0.9% for 185delAG [Struewing, J. P. et al. Natural Genetics 11:198-200 (1995)], 0.9%-1.5% for 6174delT, and 0.13% for 5382insC [Benjamin, B. et al. Natural Genetics 14:185-187 (1996); Oddoux, C. et al. Natural Genetics 14:188-190 (1996)]. In more detailed analysis shown in FIG. 3, there was a clear-cut distinction between up-regulated genes in the BRCA2 mutation carrier group and the control group. Moreover, expression patterns within the BRCA2 mutation group were highly conserved among all samples. In contrast, the gene expression profile in BRCA1 mutation carrier samples was less homogeneous, but still showed a clearly distinct gene expression pattern from that of the control group. This is exemplified in FIG. 3C where a set of genes which were down regulated in BRCA1 is displayed in comparison to both BRCA2 and control cells. In total, 137 probe sets in BRCA1 and 1345 probe sets in BRCA2 mutation carriers were differentially expressed (p≦0.05) when compared to the control samples. Using a 5% false discovery rate [Reiner, A. D. Yekutieli and Y. Benjamini. Bioinformatics 19(3):368-75 (2003)], the number of BRCA2 differentiated genes was reduced to 596. This method was not applicable for the BRCA1 group due to the lower homogeneity of the samples.

### Example 2

### Selection of Specific Genes Demonstrating Differential Expression in BRCA1/2 Carriers as Compared to Healthy Controls

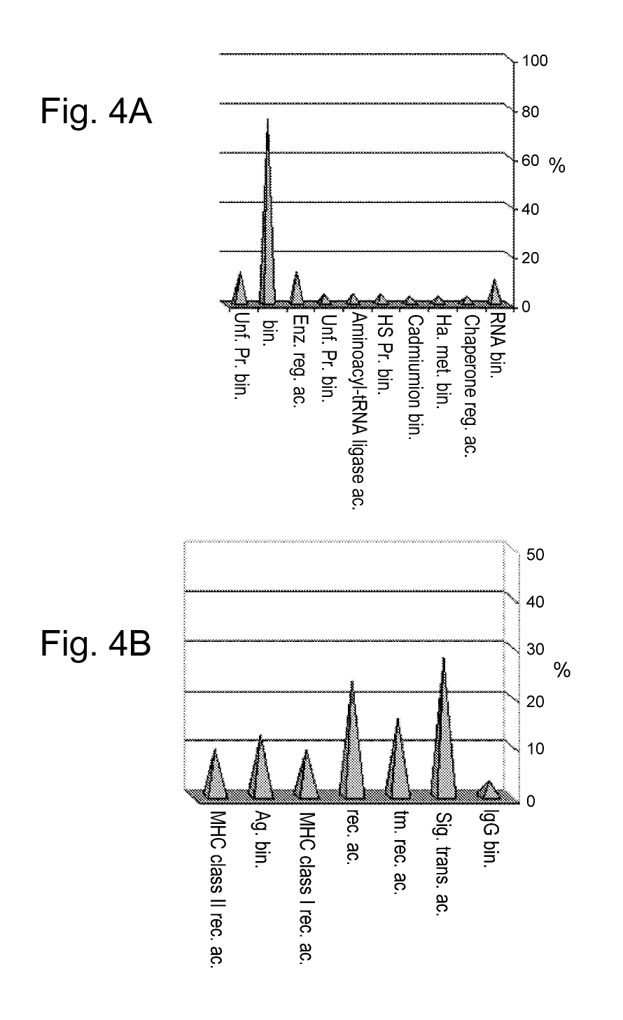

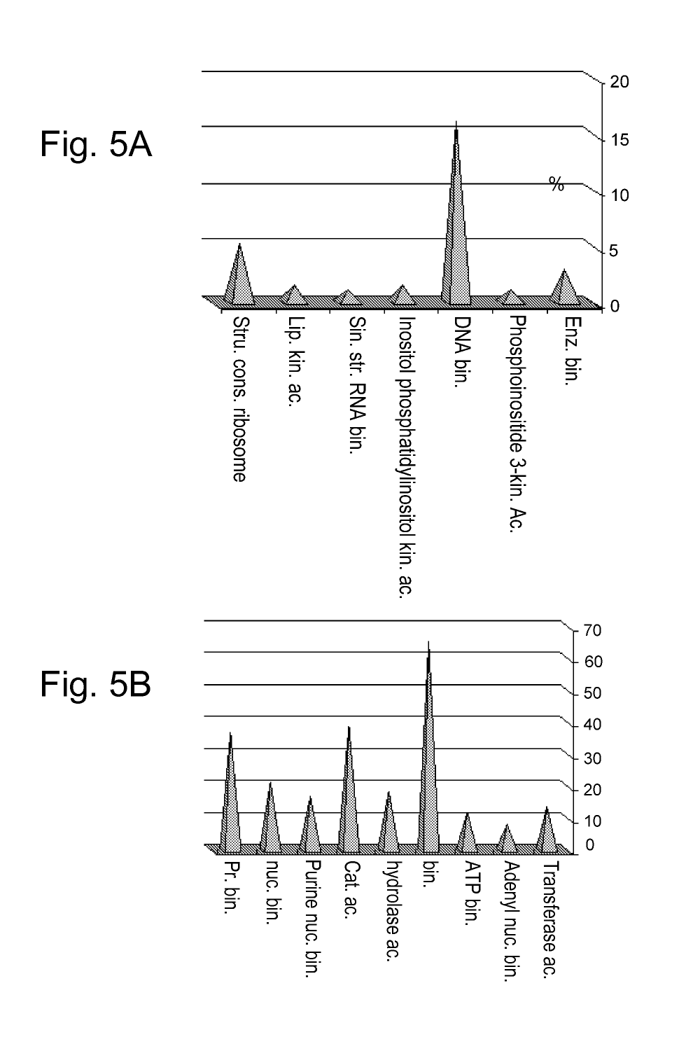

The inventors have further analyzed the results differential expression of different genes in BRCA1/2 carriers. Therefore, an additional filtration of the probe stets list was applied. The selection criterion was a signal that is differentially expressed by at least two-fold between the tested groups (BRCA1 or BRCA2 carriers vs. controls). As result of this selection, a set of 86 genes in BRCA1 carriers and 97 genes in BRCA2 carriers was established. These genes were analyzed for Gene Ontology (GO) annotations. The results for BRCA2 mutations revealed that genes related to the gene expression regulation pathways, DNA repair processes, cell cycle regulation and cancer possess the highest score. As shown by FIG. 4 (BRCA1) and FIG. 5 (BRCA2), the next largest group of genes is related to the hematological system functioning and defense system.

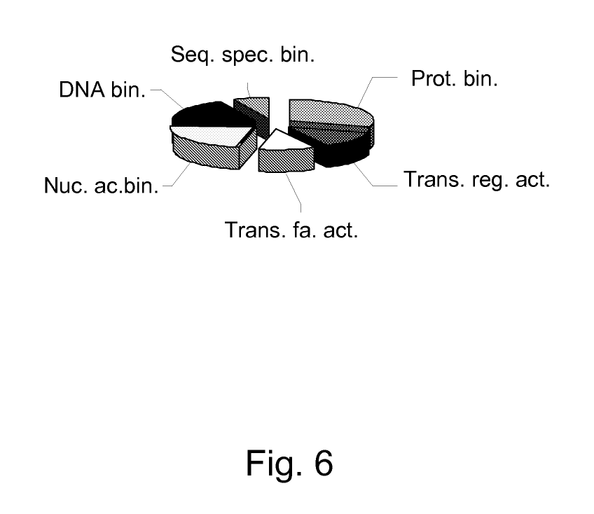

Genes expressing differentially at most samples of each of the groups were further selected (genes performing higher alterations only in a part of the patients in each group, were not chosen). From these genes only those with the most consistent pattern of expression in all samples within the same group were chosen. This selection resulted in a list of 38 genes shown in Table 2. Interestingly, the function of most of the genes which differed between the BRCA1 carrier mutation and the control group is related to transcription regulation processes and DNA binding, as illustrated by FIG. 6.

TABLE 2
List of the genes demonstrating most consistent gene expression patterns
among all the samples. These genes were demonstrated to be differentially
expressed among the tested groups using the Kruskal-Wallis test.

| | | Gene | |
|---|---|---|---|
| | | Symbol | Gene Title |
| BRAC | 1 | DNAJC12 | DnaJ (Hsp40) homolog, subfamily C, |
| 1 | | | member 12 |
| | 2 | SNX2 | Sorting nexin 2 |
| | 3 | MGC4504 | Hypothetical protein MGC4504 |
| | 4 | IFI44L | Interferon-induced protein 44-like |
| | 5 | GNLY | Granulysin |
| | 6 | SHMT2 | Serine hydroxymethyltransferase 2 |
| | | | (mitochondrial) |
| | 7 | PROSC | Proline synthetase co-transcribed homolog |
| | | | (bacterial) |
| | 8 | FBXL8 | Seryl-tRNA synthetase |
| | 9 | AUH | AU RNA binding protein/enoyl-Coenzyme |
| | | | A hydratase |
| | 10 | ANXA2 | Annexin A2 |
| | 11 | BACH2 | BTB and CNC homology 1, basic leucine |
| | | | zipper transcription factor 2 |
| | 12 | SMURF2 | SMAD specific E3 ubiquitin protein ligase 2 |
| | 13 | E2F2 | E2F transcription factor 2 |
| | 14 | HLA-DQB1 | Major histocompatibility complex, class II, |
| | | | DQ beta 1 |
| | 15 | RGS16 | Regulator of G-protein signaling 16 |
| | 16 | TNS3 | Tensin 3 |
| BRAC | 17 | LAMP2 | lysosomal-associated membrane protein 2 |
| 2 | 18 | RBL2 | Retinoblastoma-like 2 (p130) |
| | 19 | C3HC4) 7 | Membrane-associated ring finger (C3HC4) 7 |
| | 20 | NR3C1 | Nuclear receptor subfamily 3, group C, |
| | | | member 1 |
| | 21 | ELF1 | E74-like factor 1 (ets domain transcription |
| | | | factor) |
| | 22 | RPS6KB1 | RPS6KB1 |
| BRAC | 23 | TMEM30A | Transmembrane protein 30A |
| 2 | 24 | STAT5A | Signal transducer and activator of |
| | | | transcription 5A |
| | 25 | IL15RA | Interleukin 15 receptor, alpha |
| | 26 | CCNH | Cyclin H |
| | 27 | YTHDF3 | YTH domain family, member 3 |
| | 28 | STAG2 | Stromal antigen 2 |
| | 29 | RAB3GAP1 | RAB3 GTPase activating protein subunit 1 |
| | | | (catalytic) |
| | 30 | RNF11 | Ring finger protein 11 |
| | 31 | MRPS6 | Mitochondrial ribosomal protein S6 |
| | 32 | NFAT5 | Nuclear factor of activated T-cells 5, |
| | | | tonicity-responsive |
| | 33 | PKCε | Protein Kinase C epsilon |
| | 34 | NR4A2 | Nuclear receptor subfamily 4, group A, |
| | | | member 2 |
| | 35 | CCNT2 | Cyclin T2 |
| | 36 | CDKN1B | Cyclin-dependent kinase inhibitor 1B (p27, |
| | | | Kip1) |
| | 37 | MID1IP1 | MID1 interacting protein 1 (gastrulation |
| | | | specific G12-like (zebrafish)) |

### Example 3

### Real-Time RT-PCR Validation Analysis of the Selected Transcripts

The inventors next performed a real time RT-PCR analysis of those thirty-eight transcripts which were identified as being differentially expressed between the three groups (presented by Table 2). In this analysis a larger number of samples were tested: seventeen samples in the BRCA1 group, ten from the BRCA2 group and twelve samples of non-carriers of mutations. Five known housekeeping genes which were similarly expressed in the three groups were served as internal controls. In total, forty three genes were tested by TaqMan® gene cards RT-PCR. As shown by Table 3, twenty genes out of the forty tree examined, demonstrate a significantly differential expression in BRCA1 or BRCA2 mutation carriers and control samples, with the p<0.05 threshold. These genes were therefore defined as the “marker genes”. The control housekeeping genes showed no significant difference between the BRCA1/2 and non-carrier control samples. Table 4 presents a summary of the amplicon chosen for each of the marker genes, as well as partial sequences thereof.

It is worthwhile mentioning that some of the genes found by the present invention as being differentially expressed in BRCA1/2 mutation carriers are know as involve in ubiquitination. Among them are the axotrophin (MARCH7), a stem cell gene which can regulate immune tolerance and the SMURF2, which participates in TGK signaling, causes degradation of the RUNX transcription factor [Kaneki H. et al. J. Biol. Chem. 281(7):4326-33 (2006)] and induces cell senescence. Another differentially expressed gene, ELF1, was found to be downregulated in mammary cancer in mice [Hu Y. et al. Cancer Res. 64(21):7748-55 (2004)]. Other genes in this list regulate cell cycle or are DNA binding proteins.

TABLE 3
Twenty one genes that were significantly upregulated in BRCA1 and
BRCA2 mutation carriers as compared to the control group. The
P value between BRCA2 and control was calculated by Wilcoxon
Rank-Sums test (P values between BRCA1 and control are not shown.

| Gene | BRCA1/control | BRCA2/control | P VALUE |
|---|---|---|---|
| RAB3GAP1 | 2.267857 | 2.70063 | 0.0009 |
| NFAT5 | 1.820479 | 2.303268 | 0.0036 |
| MRPS6 | 2.089909 | 2.321377 | 0.0041 |
| AUH | 2.16 | 2.2 | 0.0046 |
| MID1IP1 | 1.945218 | 2.187696 | 0.0075 |
| YTHDF3 | 1.509434 | 1.957825 | 0.0145 |
| MARCH7 | 1.460844 | 1.927102 | 0.0147 |
| ELF1 | 1.75 | 2 | 0.015 |
| STAT5A | 1.48776 | 1.933504 | 0.016 |
| C6orf111 | 1.55 | 1.9 | 0.025 |
| NR3C1 | 1.356322 | 1.79716 | 0.0257 |
| NR4A2 | 1.494553 | 1.869281 | 0.0275 |
| IFI44L | 1.77931 | 1.842596 | 0.0297 |
| RPL32 | 1.54199 | 1.860155 | 0.0333 |
| SARS | 1.759104 | 1.795918 | 0.0348 |
| RP6K-1B1 | 1.494767 | 1.825334 | 0.0377 |
| CDK-1N1B | 1.68 | 1.81 | 0.041 |
| RGS16 | 1.364034 | 1.736471 | 0.0463 |
| DNAJC12 | 1.68 | 1.71 | 0.0491 |
| EIF3S7 | 1.57 | 1.93 | 0.0491 |
| SMURF2 | 1.487574 | 1.782943 | 0.0491 |

TABLE 4
list of marker genes differentially expressed
in carriers of BRCA1/2 gee mutations

| | | | | | | | Partial |
|---|---|---|---|---|---|---|---|
| | Gene | Gene | | Amplicon | Exon | Assay | sequence of |
| Number | Symbol | name | RefSeq | length | boundary | location | replicon |
| 1 | RAB3GAP1 | RAB3 | NM_012233.1 | 85 | 23-24 | 2738 | GAATGCCCAGAGGGC |
| | | GTPase | SEQ ID NO. 1 | | | | TGCAGCTATG |
| | | activating | | | | | SEQ ID NO. 25 |
| | | protein | | | | | |
| | | subunit 1 | | | | | |
| | | (catalytic) | | | | | |
| 2 | NFAT5 | nuclear | NM_006599 | 70 | 5-6 | 2352 | GACACTGGCGGTGGA |
| | | factor of | SEQ ID NO. 2 | | | | CTGCGTAGGG |
| | | activated | | | | | SEQ ID NO. 26 |
| | | T-cells 5, | | | | | |
| | | tonicity- | | | | | |
| | | responsive | | | | | |
| 3 | MRPS6 | mitochondrial | NM_032476.2 | 100 | 2-3 | 359 | GCAGCACAACAGAGG |
| | | ribosomal | SEQ ID NO. 3 | | | | CGGGTATTTC |
| | | protein S6 | | | | | SEQ ID NO. 27 |
| 4 | AUH | AU RNA | NM_001698 | 61 | 7-8 | 878 | TTTTTACCTCAGGGAC |
| | | binding | SEQ ID NO. 4 | | | | CTGTTGCAA |
| | | protein/ | | | | | SEQ ID NO. 28 |
| | | enoyl- | | | | | |
| | | Coenzyme | | | | | |
| | | A | | | | | |
| | | hydratase | | | | | |
| 5 | MID1IP1 | MID1 | NM_021242 | 70 | 1-2 | 596 | AGAGGAGGCCAGGGC |
| | | interacting | SEQ ID NO. 5 | | | | TCGACCCACA |
| | | protein 1 | | | | | SEQ ID NO. 29 |
| | | (gastrulation | | | | | |
| | | specific | | | | | |
| | | G12 | | | | | |
| | | homolog | | | | | |
| | | (zebrafish)) | | | | | |
| 6 | RGS16 | regulator | NM_002928.3 | 108 | 1-2 | 203 | TGCCTGGAGAGAGCC |
| | | of G- | SEQ ID NO. 6 | | | | AAAGAGTTCA |
| | | protein | | | | | SEQ ID NO. 30 |
| | | signaling | | | | | |
| | | 16 | | | | | |
| 7 | MARCH7 | membrane- | NM_022826.2 | 102 | 5-6 | 1738 | AAAAGAGAGCCTCCT |
| | | associated | SEQ ID NO. 7 | | | | TTTAGAGGAC |
| | | ring finger | | | | | SEQ ID NO. 31 |
| | | (C3HC4) 7 | | | | | |
| 8 | NR3C1 | nuclear | X03348 | 73 | 4-5 | 1602 | AATGAACCTGGAAGC |
| | | receptor | SEQ ID NO. 8 | | | | TCGAAAAACA |
| | | subfamily | | | | | SEQ ID NO. 32 |
| | | 3, group | | | | | |
| | | C, member 1 | | | | | |
| | | (glucocorticoid | | | | | |
| | | receptor) | | | | | |
| 9 | ELF1 | E74-like | NM_172373.2 | 76 | 1-2 | 301 | GGATGAACGACAGCT |
| | | factor 1 | SEQ ID NO. 9 | | | | TGGTGATCCA |
| | | (ets | | | | | SEQ ID NO. 33 |
| | | domain | | | | | |
| | | transcription | | | | | |
| | | factor) | | | | | |
| 10 | RPS6KB1 | ribosomal | NM_003161.2 | 97 | 6-7 | 690 | AAGACACTGCCTGCTT |
| | | protein S6 | SEQ ID NO. 10 | | | | TTACTTGGC |
| | | kinase, | | | | | SEQ ID NO. 34 |
| | | 70 kDa, | | | | | |
| | | polypeptide 1 | | | | | |
| 11 | STAT5A | signal | NM_003152.2 | 85 | 17-18 | 2706 | ACTCCTGTGCTGGCTA |
| | | transducer | SEQ ID NO. 11 | | | | AAGCTGTTG |
| | | and | | | | | SEQ ID NO. 35 |
| | | activator | | | | | |
| | | of | | | | | |
| | | transcription | | | | | |
| | | 5A | | | | | |
| 12 | YTHDF3 | YTH | NM_152758.4 | 118 | 4-5 | 2044 | GGAAGCCATGCGTAG |
| | | domain | SEQ ID NO. 12 | | | | GGAGAGAAAT |
| | | family, | | | | | SEQ ID NO. 36 |
| | | member 3 | | | | | |
| 13 | DNAJC12 | DnaJ | NM_021800 | 82 | 3-4 | 467 | CAGTGAAGACGTCAA |
| | | (Hsp40) | SEQ ID NO. 13 | | | | TGCACTGGGT |
| | | homolog, | | | | | SEQ ID NO. 37 |
| | | subfamily | | | | | |
| | | C, member | | | | | |
| | | 12 | | | | | |
| 14 | IFI44L | interferon- | NM_006820 | 124 | 4-5 | 900 | CATAACCGAGCGGTA |
| | | induced | SEQ ID NO. 14 | | | | TAGGATATAT |
| | | protein 44- | | | | | SEQ ID NO. 38 |
| | | like | | | | | |
| 15 | SARS | seryl- | NM_006513 | 101 | 1-2 | 216 | GCGACGATGTAGATT |
| | | tRNA | SEQ ID NO. 15 | | | | TCGGGCAGAC |
| | | synthetase | | | | | SEQ ID NO. 39 |
| 16 | SMURF2 | SMAD | NM_022739.3 | 100 | 7-8 | 960 | GGAGCGCCCAACACG |
| | | specific E3 | SEQ ID NO. 16 | | | | ACCGGCATCC |
| | | ubiquitin | | | | | SEQ ID NO. 40 |
| | | protein | | | | | |
| | | ligase 2 | | | | | |
| 17 | SFRS18 | splicing | NM_032870.2 | 56 | 3-4 | 316 | CAGGATCCAAGCCAG |
| | (C6ORF111) | factor, | SEQ ID NO. 17 | | | | ATTGATTGGG |
| | | arginine/ | | | | | SEQ ID NO. 41 |
| | | serine-rich | | | | | |
| | | 18 | | | | | |
| 18 | NR4A2 | nuclear | NM_006186 | 69 | 5-6 | 1491 | TGGACTATTCCAGGTT |
| | | receptor | SEQ ID NO. 18 | | | | CCAGGCGAA |
| | | subfamily | | | | | SEQ ID NO. 42 |
| | | 4, group A, | | | | | |
| | | member 2 | | | | | |
| 19 | CDKN1B | cyclin- | BC001971 | 71 | 1-2 | 857 | TGCAACCGACGATTCT |
| | | dependent | SEQ ID NO. 19 | | | | TCTACTCAA |
| | | kinase | | | | | SEQ ID NO. 43 |
| | | inhibitor | | | | | |
| | | 1B (p27, | | | | | |
| | | Kip1) | | | | | |
| 20 | EIF3D | eukaryotic | NM_003753.3 | 132 | 12-13 | 1367 | GAGTGGGATTCCAGG |
| | | translation | SEQ ID NO. 20 | | | | CACTGTAATG |
| | | initiation | | | | | SEQ ID NO. 44 |
| | | factor 3, | | | | | |
| | | subunit D | | | | | |
| 21 | 18S | Eukaryotic | X03205.1 | 187 | | 609 | TGGAGGGCAAGTCTG |
| | | 18S rRNA | SEQ ID NO. 21 | | | | GTGCCAGCAG |
| | | | | | | | SEQ ID NO. 45 |
| 22 | RPS9 | ribosomal | NM_001013.3 | 156 | 4-5 | 467 | GCGCCATATCAGGGT |
| | | protein S9 | SEQ ID NO. 22 | | | | CCGCAAGCAG |
| | | | | | | | SEQ ID NO. 46 |
| 23 | ACTB | actin, beta | NM_001101.2 | 171 | 1-1 | 49 | CCTTTGCCGATCCGCC |
| | | | SEQ ID NO. 23 | | | | GCCCGTCCA |
| | | | | | | | SEQ ID NO. 47 |
| 24 | HSPCB | heat shock | NM_007355.2 | 155 | 11-12 | 2142 | GCATGATCAAGCTAG |
| | | protein | SEQ ID NO. 24 | | | | GTCTAGGTAT |
| | | 90 kDa | | | | | SEQ ID NO. 48 |
| | | alpha | | | | | |
| | | (cytosolic), | | | | | |
| | | class B | | | | | |
| | | member 1 | | | | | |

### Example 4

### GO Analysis of Differentially Expressed Genes Between BRCA1/2 Mutation Carriers Versus Non-Carriers

Gene Ontology analysis was performed on a list of genes which had different expression patterns for either the BRCA1 or the BRCA2 groups as compared to the control group. Analysis in the BRCA2 mutation group, revealed that most of the genes are related to gene expression regulation pathways involved in DNA repair processes (i.e. DNAJ, RAD51), cell cycle regulation (i.e. cyclin H, Kip1), cancer associated (i.e. RPS6KB1, RBL2) and apoptosis. Furthermore, a number of these genes were shown to function together (for example SMURF2 and RNF11). Mutations in BRCA1 have been shown to impair the homologous repair of double stranded breaks in the DNA, and the BRCA1 protein has been shown to function in cell cycle regulation. Therefore, these results might be relevant to the function of BRCA1 and BRCA2. The next largest group of genes is related to the hematological system functioning and the immune system (i.e. HLA-DQB1, Granulysin), as can be expected when tested in lymphocytes. It have been previously shown that BRCA1 regulates targets of the innate immune system dependent on IFN signaling [Buckley, N. E. et al. Mol. Cancer Res. 5(3):261-70 (2007)].

TABLE 5
Genes differentially expressed (with p < 0.05 and >1.5 fold) in BRCA1
gene mutation carriers.

| | Representative | |
|---|---|---|
| Affimetrix ID | Public ID | Gene Title |
| 204972_at | NM_016817 | 2′-5′-oligoadenylate synthetase 2, 69/71 kDa |
| 202672_s_at | NM_001674 | activating transcription factor 3 |
| 222108_at | AC004010 | adhesion molecule with Ipg-like domain 2 |
| 201000_at | NM_001605 | alanyl-tRNA synthetase |
| 213503_x_at | BE908217 | annexin A2 |
| 201590_x_at | NM_004039 | annexin A2 |
| 201525_at | NM_001647 | apolipoprotein D |
| 203747_at | NM_004925 | aquaporin 3 |
| 205047_s_at | NM_001673 | asparagine synthetase |
| 211852_s_at | AF106861 | attractin |
| 211725_s_at | BC005884 | BH3 interacting domain death agonist; BH3 interacting |
| | | domain death agonist |
| 211190_x_at | AF054817 | CD84 antigen (leukocyte antigen) |
| 218085_at | NM_015961 | chromatin modifying protein 5 |
| 220235_s_at | NM_018372 | chromosome 1 open reading frame 103 |
| 206707_x_at | NM_015864 | chromosome 6 open reading frame 32 |
| 218325_s_at | NM_022105 | death associated transcription factor 1 |
| Systematic | Genbank | Description |
| 222154_s_at | AK002064 | DNA polymerase-transactivated protein 6 |
| 200880_at | AL534104 | DnaJ (Hsp40) homolog, subfamily A, member 1 |
| 200881_s_at | AL534104 | DnaJ (Hsp40) homolog, subfamily A, member 1 |
| 209015_s_at | BC002446 | DnaJ (Hsp40) homolog, subfamily B, member 6 |
| 219551_at | NM_018456 | ELL associated factor 2 |
| 37145_at | M85276 | Granulysin |
| 206976_s_at | NM_006644 | heat shock 105 kDa/110 kDa protein 1 |
| 208744_x_at | D86956 | heat shock 105 kDa/110 kDa protein 1 |
| 200799_at | NM_005345 | heat shock 70 kDa protein 1A |
| 200800_s_at | NM_005345 | heat shock 70 kDa protein 1A; heat shock 70 kDa |
| | | protein 1B |
| 202581_at | NM_005346 | heat shock 70 kDa protein 1B |
| 211968_s_at | AI962933 | heat shock 90 kDa protein 1, alpha |
| 215933_s_at | Z21533 | hematopoietically expressed homeobox |
| 220387_s_at | NM_007071 | HERV-H LTR-associating 3 |
| 211597_s_at | AB059408 | homeodomain-only protein; homeodomain-only |
| | | protein |
| 203914_x_at | NM_000860 | hydroxyprostaglandin dehydrogenase 15-(NAD) |
| 205404_at | NM_005525 | hydroxysteroid (11-beta) dehydrogenase 1 |
| 213674_x_at | AI858004 | immunoglobulin heavy constant delta |
| 214973_x_at | AJ275469 | immunoglobulin heavy constant delta |
| 211798_x_at | AB001733 | immunoglobulin lambda joining 3 |
| 211881_x_at | AB014341 | immunoglobulin lambda joining 3 |
| 205786_s_at | NM_000632 | integrin, alpha M (complement component |
| | | receptor 3, alpha; also known as CD11b (p170), |
| | | macrophage antigen alpha polypeptide); |
| | | integrin, alpha M (complement component |
| | | receptor 3, alpha; also known as CD11b (p170), |
| | | macrophage antigen alpha polypeptide) |
| 219209_at | NM_022168 | interferon induced with helicase C domain 1 |
| 208436_s_at | NM_004030 | interferon regulatory factor 7 |
| 202220_at | NM_014949 | KIAA0907 |
| 212714_at | AL050205 | La ribonucleoprotein domain family, member 4 |
| 221274_s_at | NM_030805 | lectin, mannose-binding 2-like; lectin, |
| | | mannose-binding 2-like |
| 205569_at | NM_014398 | lysosomal-associated membrane protein 3 |
| 209199_s_at | L08895 | MADS box transcription enhancer factor 2, |
| | | polypeptide C |
| | | (myocyte enhancer factor 2C) |
| 209200_at | AL536517 | MADS box transcription enhancer factor 2, |
| | | polypeptide C |
| | | (myocyte enhancer factor 2C) |
| 213537_at | AI128225 | major histocompatibility complex, class II, DP |
| | | alpha 1 |
| 209823_x_at | M17955 | major histocompatibility complex, class II, DQ |
| | | beta 1 |
| 208306_x_at | NM_021983 | Major histocompatibility complex, class II, DR |
| | | beta 3 |
| 201475_x_at | NM_004990 | methionine-tRNA synthetase |
| 213733_at | BF740152 | myosin IF |
| 210218_s_at | U36501 | nuclear antigen Sp100 |
| 219165_at | NM_021630 | PDZ and LIM domain 2 (mystique) |
| 204286_s_at | NM_021127 | phorbol-12-myristate-13-acetate-induced |
| | | protein 1 |
| 210617_at | U87284 | phosphate regulating endopeptidase homolog, |
| | | X-linked (hypophosphatemia, vitamin D |
| | | resistant rickets) |
| 201397_at | NM_006623 | phosphoglycerate dehydrogenase |
| 202446_s_at | AI825926 | phospholipid scramblase 1 |
| 202430_s_at | NM_021105 | phospholipid scramblase 1 |
| 220892_s_at | NM_021154 | phosphoserine aminotransferase 1 |
| 205267_at | NM_006235 | POU domain, class 2, associating factor 1 |
| 201703_s_at | NM_002714 | protein phosphatase 1, regulatory subunit 10 |
| 219412_at | NM_022337 | RAB38, member RAS oncogene family |
| 212125_at | NM_002883 | Ran GTPase activating protein 1 |
| 214369_s_at | AI688812 | RAS guanyl releasing protein 2 (calcium and |
| | | DAG-regulated) |
| 206220_s_at | NM_007368 | RAS p21 protein activator 3 |
| 209325_s_at | U94829 | regulator of G-protein signaling 16 |
| 213566_at | NM_005615 | ribonuclease, RNase A family, k6; ribonuclease, |
| | | RNase A family, k6 |
| 213502_x_at | AA398569 | similar to bK246H3.1 (immunoglobulin lambda- |
| | | like polypeptide 1, |
| | | pre-B-cell specific) |
| 213820_s_at | T54159 | START domain containing 5 |
| 209999_x_at | AB005043 | suppressor of cytokine signaling 1 |
| 209307_at | AB014540 | SWAP-70 protein |
| 216180_s_at | AK026758 | synaptojanin 2 |
| 222010_at | BF224073 | t-complex 1 |
| 220558_x_at | NM_005705 | tetraspanin 32 |
| 210176_at | AL050262 | toll-like receptor 1 |
| 200629_at | NM_004184 | tryptophanyl-tRNA synthetase |
| 213361_at | AW129593 | tudor domain containing 7 |
| 201535_at | NM_007106 | ubiquitin-like 3 |
| 206133_at | NM_017523 | XIAP associated factor-1 |

TABLE 6
Genes differentially expressed (with p < 0.05 and >2 fold) in BRCA2 gene
mutation carriers.

| Affimetrix | Representative | |
|---|---|---|
| ID | Public ID | Gene Title |
| 201963_at | NM_021122 | acyl-CoA synthetase long-chain family member 1 |
| 208002_s_at | NM_007274 | acyl-CoA thioesterase 7 |
| 215728_s_at | AL031848 | acyl-CoA thioesterase 7 |
| 200734_s_at | BG341906 | ADP-ribosylation factor 3 |
| 211622_s_at | M33384 | ADP-ribosylation factor 3; ADP-ribosylation factor 3 |
| 221589_s_at | AW612403 | Aldehyde dehydrogenase 6 family, member A1 |
| 208859_s_at | AI650257 | alpha thalassemia/mental retardation syndrome X- |
| | | linked (RAD54 homolog, S. cerevisiae) |
| 203566_s_at | NM_000645 | amylo-1,6-glucosidase, 4-alpha-glucanotransferase |
| | | (glycogen debranching enzyme, glycogen storage |
| | | disease type III) |
| 200602_at | NM_000484 | amyloid beta (A4) precursor protein (peptidase nexin- |
| | | II, Alzheimer disease) |
| 213106_at | AI769688 | ATPase, aminophospholipid transporter (APLT), Class |
| | | I, type 8A, member 1 |
| 207521_s_at | AF068220 | ATPase, Ca++ transporting, ubiquitous |
| 201242_s_at | BC000006 | ATPase, Na+/K+ transporting, beta 1 polypeptide |
| 203140_at | NM_001706 | B-cell CLL/lymphoma 6 (zinc finger protein 51); B-cell |
| | | CLL/lymphoma 6 (zinc finger protein 51) |
| 221478_at | AL132665 | BCL2/adenovirus E1B 19 kDa interacting protein 3- |
| | | like; BCL2/adenovirus E1B 19 kDa interacting protein |
| | | 3-like |
| 218090_s_at | NM_018117 | bromodomain and WD repeat domain containing 2 |
| 214450_at | NM_001335 | cathepsin W (lymphopain); cathepsin W (lymphopain) |
| 218871_x_at | NM_018590 | chondroitin sulfate GalNAcT-2 |
| 205583_s_at | NM_024810 | Chromosome X open reading frame 45 |
| 205518_s_at | NM_003570 | cytidine monophosphate-N-acetylneuraminic acid |
| | | hydroxylase (CMP-N-acetylneuraminate |
| | | monooxygenase) |
| 221628_s_at | AF326966 | cytokine-like nuclear factor n-pac |
| 213998_s_at | AW188131 | DEAD (Asp-Glu-Ala-Asp) box polypeptide 17 |
| 212107_s_at | BE561014 | DEAH (Asp-Glu-Ala-His) box polypeptide 9 |
| Systematic | Genbank | Description |
| 204646_at | NM_000110 | dihydropyrimidine dehydrogenase |
| 219237_s_at | NM_024920 | DnaJ (Hsp40) homolog, subfamily B, member 14 |
| 201693_s_at | NM_001964 | early growth response 1 |
| 206115_at | NM_004430 | early growth response 3 |
| 209004_s_at | AF142481 | F-box and leucine-rich repeat protein 5 |
| 201540_at | NM_001449 | four and a half LIM domains 1 |
| 206492_at | NM_002012 | fragile histidine triad gene |
| 215001_s_at | AL161952 | glutamate-ammonia ligase (glutamine synthetase) |
| 208798_x_at | AF204231 | golgi autoantigen, golgin subfamily a, 8A |
| 212525_s_at | AA760862 | H2A histone family, member X |
| 202979_s_at | NM_021212 | HCF-binding transcription factor Zhangfei |
| 213359_at | W74620 | Heterogeneous nuclear ribonucleoprotein D (AU-rich |
| | | element RNA binding protein 1, 37 kDa) |
| 214753_at | AW084068 | Hypothetical gene CG012 |
| 221899_at | AI809961 | Hypothetical gene CG012 |
| 213375_s_at | N80918 | Hypothetical gene CG018 |
| 218051_s_at | NM_022908 | Hypothetical protein FLJ12442 |
| 213212_x_at | AI632181 | Hypothetical protein LOC161527 |
| 213931_at | AI819238 | inhibitor of DNA binding 2, dominant negative helix- |
| | | loop-helix protein; inhibitor of DNA binding 2B, |
| | | dominant negative helix-loop-helix protein |
| 203607_at | NM_014937 | inositol polyphosphate-5-phosphatase F |
| 203628_at | H05812 | insulin-like growth factor 1 receptor |
| 38892_at | D87077 | KIAA0240 |
| 203049_s_at | NM_014639 | KIAA0372 |
| 207719_x_at | NM_014812 | KIAA0470 |
| 212633_at | AL132776 | KIAA0776 |
| 218219_s_at | NM_018697 | LanC lantibiotic synthetase component C-like 2 |
| | | (bacterial) |
| 205668_at | NM_002349 | lymphocyte antigen 75 |
| 213975_s_at | AV711904 | lysozyme (renal amyloidosis); leukocyte |
| | | immunoglobulin-like receptor, subfamily B (with TM |
| | | and ITIM domains), member 1 |
| 220615_s_at | NM_018099 | male sterility domain containing 1 |
| 201755_at | NM_006739 | MCM5 minichromosome maintenance deficient 5, cell |
| | | division cycle 46 (S. cerevisiae) |
| 213158_at | BG251521 | MRNA; cDNA DKFZp586B211 (from clone |
| | | DKFZp586B211) |
| 201467_s_at | AI039874 | NAD(P)H dehydrogenase, quinone 1 |
| 205005_s_at | AW293531 | N-myristoyltransferase 2 |
| 205006_s_at | NM_004808 | N-myristoyltransferase 2 |
| 201577_at | NM_000269 | non-metastatic cells 1, protein (NM23A) expressed in |
| 216321_s_at | X03348 | nuclear receptor subfamily 3, group C, member 1 |
| | | (glucocorticoid receptor) |
| 207564_x_at | NM_003605 | O-linked N-acetylglucosamine (GlcNAc) transferase |
| | | (UDP-N-acetylglucosamine:polypeptide-N- |
| | | acetylglucosaminyl transferase) |
| 201246_s_at | NM_017670 | OTU domain, ubiquitin aldehyde binding 1 |
| 201490_s_at | NM_005729 | peptidylprolyl isomerase F (cyclophilin F) |
| 209422_at | AY027523 | PHD finger protein 20 |
| 218640_s_at | BF439250 | pleckstrin homology domain containing, family F (with |
| | | FYVE domain) member 2 |
| 207002_s_at | NM_002656 | pleiomorphic adenoma gene-like 1 |
| 222273_at | AI419423 | poly(A) polymerase gamma |
| 212016_s_at | AA679988 | Polypyrimidine tract binding protein 1 |
| 211791_s_at | AF044253 | potassium voltage-gated channel, shaker-related |
| | | subfamily, beta member 2 |
| 201300_s_at | NM_000311 | prion protein (p27-30) (Creutzfeld-Jakob disease, |
| | | Gerstmann-Strausler-Scheinker syndrome, fatal |
| | | familial insomnia) |
| 208988_at | BE675843 | PRO1880 protein |
| 218668_s_at | NM_021183 | RAP2C, member of RAS oncogene family |
| 221524_s_at | AL138717 | Ras-related GTP binding D |
| 209285_s_at | N38985 | retinoblastoma-associated protein 140 |
| 205407_at | NM_021111 | reversion-inducing-cysteine-rich protein with kazal |
| | | motifs |
| 201167_x_at | NM_004309 | Rho GDP dissociation inhibitor (GDI) alpha |
| 213350_at | BF680255 | Ribosomal protein S11 |
| 209889_at | AF274863 | SEC31-like 2 (S. cerevisiae) |
| 201996_s_at | AL524033 | spen homolog, transcriptional regulator (Drosophila) |
| 203455_s_at | NM_002970 | spermidine/spermine N1-acetyltransferase |
| 210592_s_at | M55580 | spermidine/spermine N1-acetyltransferase |
| 210172_at | D26121 | splicing factor 1 |
| 215113_s_at | AK000923 | SUMO1/sentrin/SMT3 specific peptidase 3 |
| 213510_x_at | AW194543 | TL132 protein |
| 212983_at | NM_005343 | v-Ha-ras Harvey rat sarcoma viral oncogene homolog |
| 209348_s_at | BF508646 | v-maf musculoaponeurotic fibrosarcoma oncogene |
| | | homolog (avian) |
| 220118_at | NM_014383 | zinc finger and BTB domain containing 32 |
| 203739_at | NM_006526 | zinc finger protein 217 |
| 212774_at | AJ223321 | zinc finger protein 238 |
| 221645_s_at | M27877 | zinc finger protein 83 (HPF1) |
| 214670_at | AA653300 | zinc finger with KRAB and SCAN domains 1 |

In summary, identification of marker genes differentially expressed in carriers of BRCA1/2 gene mutations as compared to non-carrier controls, by the present invention, demonstrate the feasibility of using such marker genes or any combination thereof in the diagnosis of carriers.

### Example 5

### Establishment of a Predictive Marker Set and Associated Expression Cutoff Values for BRCA1/BRCA2 Mutations

In order to refine the set of markers for detection of BRCA1 and BRCA2 mutations that predispose subjects to breast, ovarian and other cancers, the eighteen candidate marker genes which displayed the most statistically significant differential expression out of the twenty candidate marker genes presented in Table 4 were further analyzed. These genes were: MRPS6, CDKN1B, ELF1, NFAT5, NR3C1, SARS, SMURF2, STAT5A, YTHDF3, AUH, EIF3D, IFI44L, MARCH7, MID1IP1, NR4A2, RAB3GAP1, RGS16 and SFRS18 (C6orf111). Twenty-one female carriers of either BRCA1 or BRCA2 mutations, or both, and nineteen normal females were chosen for RT-PCR validation analysis. One mutation carrier and one normal subject were disqualified due to low grade RT-PCR products that did not allow reliable interpretation, leaving twenty mutation carriers (13 BRCA1, 6 BRCA2 and one BRCA 1+2 carriers) and eighteen normal subjects. For normalization of expression values and internal calibration, mRNA transcribed from the gene encoding ribosomal protein S9 (RPS9), a ubiquitously-as well as consistently-expressed gene that is free of pseudogenes was used. For comparison of normalized expression values between different samples, or relative quantitation, the following equation was used:

RQ (Relative Quantitation)=2^{-ΔCt}

### Where:

ΔCt=(CT, X)−(CT, R); the difference in threshold cycles for target and reference
CT, X=threshold cycle for test sample gene amplification
CT, R=threshold cycle for reference (control sample) gene amplification, said threshold cycle being an RT-PCR cycle number that produces sufficient luminescent product to exceed a specific luminosity value.

For the analysis of sensitivity-specificity relation of the assay, an ROC curve was constructed, and the area under this curve was calculated.

A P value of <0.05 was considered significant. Using a receiver-operator characteristic plot (ROC) analysis of the results, presented in Table 7, the candidate marker gene group was restricted to thirteen genes presented in Table 8. Furthermore, since all of the chosen genes displayed lower expression levels in mutation carriers, a cutoff value was calculated for each gene as presented in Table 8, below which threshold each gene was assigned the value of “1”, indicating a possible mutation carrier, and above which “0”, indicating a possible non-carrier. The cutoff value was optimized using ROC such that it would produce maximal accuracy, i.e., an optimal combination of sensitivity and specificity to the predicted mutations.

Using the selected markers, threshold values and the given experiment population, it was calculated that any combination of six positive (“1”) markers accumulated in a sample from a single subject is indicative of a mutation in either BRCA1, BRCA2 or both in said subject, with a sensitivity of 90%, a specificity of 84%, a positive predictive value of 85.7% and a negative predictive value of 88.2%. The experimental population marker expression values are given in Table 9, and their marker indices according to the thresholds of Table 8 are given in Table 10.

TABLE 7
ROC calculation of gene expression threshold
Area Under the Curve

| | | | | Asymptotic 95% | |
|---|---|---|---|---|---|
| | | | | Confidence | |
| | | | | Interval | |
| | | Std. | Asymptotic | Lower | Upper |
| Gene | Area | Error^{a} | Sig.^{b} | Bound | Bound |
| MRPS6 | .878 | .060 | .000 | .760 | .996 |
| AUH | .663 | .093 | .087 | .481 | .844 |
| CDKN1B | .781 | .082 | .003 | .619 | .942 |
| IFI44L | .653 | .097 | .108 | .463 | .842 |
| MARCH7 | .592 | .094 | .335 | .407 | .776 |
| MID1IP1 | .629 | .094 | .174 | .444 | .814 |
| NFAT5 | .814 | .072 | .001 | .672 | .956 |
| NR3C1 | .753 | .085 | .008 | .585 | .920 |
| NR4A2 | .658 | .091 | .096 | .481 | .836 |
| RAB3GAP1 | .692 | .088 | .044 | .519 | .864 |
| RGS16 | .681 | .094 | .057 | .497 | .864 |
| RPS6KB1 | .447 | .095 | .579 | .261 | .634 |
| SARS | .769 | .085 | .005 | .603 | .936 |
| SFRS18 | .719 | .089 | .021 | .546 | .893 |
| (C6orf111) | | | | | |
| SMURF2 | .749 | .083 | .009 | .585 | .912 |
| STAT5A | .800 | .074 | .002 | .656 | .944 |
| YTHDF | .783 | .076 | .003 | .634 | .933 |
| ELF1 | .750 | .083 | .009 | .588 | .912 |

^{a}Standard error under the nonparametric assumption
^{b}Null hypothesis: true area under curve for ROC curve = 0.5

TABLE 8
Final prognostic marker panel and corresponding expression thresholds.

| | Area under | Cut off | | |
|---|---|---|---|---|
| | the ROC | value | | |
| Gene | curve | by ROC | Specificity | Sensitivity |
| MRPS6 | 0.878 | 0.094685771 | 0.889 | 0.6 |
| CDKN1B | 0.781 | 0.106924191 | 0.778 | 0.8 |
| ELF1 | 0.75 | 0.230392832 | 0.778 | 0.65 |
| NFAT5 | 0.814 | 0.038620236 | 0.833 | 0.7 |
| NR3C1 | 0.753 | 0.077261802 | 0.778 | 0.85 |
| SARS | 0.769 | 0.194105442 | 0.778 | 0.75 |
| SMURF2 | 0.749 | 0.026263438 | 0.722 | 0.75 |
| STAT5A | 0.8 | 0.042452737 | 0.778 | 0.5 |
| YTHDF3 | 0.783 | 0.038169237 | 0.778 | 0.65 |
| AUH | 0.663 | 0.007434947 | 0.667 | 0.65 |
| EIF3D | 0.553 | 0.235698204 | 0.556 | 0.5 |
| IFI44L | 0.653 | 0.054651946 | 0.667 | 0.75 |
| NR4A2 | 0.658 | 0.007216956 | 0.667 | 0.45 |

Assuming that the sampled population represents the general population, a combination of at least six marker genes having expression values different than the above-presented cutoff values predicts the presence of a mutation in at least one of BRCA1 and BRCA2 in the tested subject, said prediction given with a sensitivity of 90%, a specificity of 84%, a positive predictive value of 85.7% and a negative predictive value of 88.2%.

TABLE 9 (a)
RT-PCR expression values (relative values) for MRPS6, CDKN1B, ELF1,
NFAT5, NR3C1, SARS and SMURF2 expression values.
(a)

| Sample | Mutation | MRPS6 | CDKN1B | ELF1 | NFAT5 | NR3C1 | SARS | SMURF2 |
|---|---|---|---|---|---|---|---|---|
| b1 | BRCA1 185delAG | 0.115263 | 0.08139 | 0.22688 | 0.046071 | 0.066939 | 0.189465 | 0.024501 |
| b12 | BRCA1 185delAG | 0.097666 | 0.087474 | 0.268129 | 0.036448 | 0.054372 | 0.150935 | 0.022483 |
| b13 | BRCA1 185delAG | 0.101955 | 0.073966 | 0.15368 | 0.0426 | 0.071744 | 0.110338 | 0.030165 |
| b14 | BRCA1 185delAG | 0.136219 | 0.163799 | 0.390935 | 0.057832 | 0.112189 | 0.354044 | 0.030649 |
| b15 | BRCA1 185delAG | 0.090622 | 0.098755 | 0.251739 | 0.033285 | 0.057392 | 0.168404 | 0.023131 |
| b2 | BRCA1 185delAG | 0.075572 | 0.065064 | 0.124654 | 0.02253 | 0.039146 | 0.10555 | 0.01385 |
| b21 | BRCA1 185delAG | 0.087717 | 0.102309 | 0.225156 | 0.039173 | 0.061982 | 0.180491 | 0.024586 |
| b3 | BRCA1 185delAG | 0.087535 | 0.077805 | 0.220982 | 0.022189 | 0.051119 | 0.230526 | 0.014968 |
| b9 | BRCA1 185delAG | 0.097869 | 0.090622 | 0.181495 | 0.036855 | 0.047235 | 0.117034 | 0.027375 |
| b7 | BRCA1 185delAG | 0.119493 | 0.067172 | 0.159762 | 0.016851 | 0.046327 | 0.125521 | 0.018685 |
| b8 | BRCA1 185delAG | 0.092783 | 0.097193 | 0.26554 | 0.025155 | 0.074068 | 0.224845 | 0.025737 |
| b16 | BRCA1 5382insC | 0.088757 | 0.203204 | 0.355766 | 0.070609 | 0.17374 | 0.167241 | 0.052338 |
| b5 | BRCA1 5382insC | 0.096924 | 0.09855 | 0.204901 | 0.027375 | 0.068678 | 0.204051 | 0.023585 |
| b10 | BRCA2 6174delT | 0.074017 | 0.096857 | 0.189071 | 0.030777 | 0.033032 | 0.130308 | 0.019791 |
| b11 | BRCA2 6174delT | 0.047762 | 0.103593 | 0.194117 | 0.031163 | 0.063725 | 0.141414 | 0.018998 |
| b17 | BRCA2 6174delT | 0.102949 | 0.100481 | 0.236678 | 0.037473 | 0.061044 | 0.173619 | 0.017494 |
| b18 | BRCA2 6174delT | 0.08094 | 0.075572 | 0.167938 | 0.034506 | 0.048161 | 0.13167 | 0.017936 |
| b19 | BRCA2 6174delT | 0.090747 | 0.131944 | 0.299993 | 0.05954 | 0.108292 | 0.22751 | 0.031533 |
| b20 | BRCA2 6174delT | 0.078059 | 0.111854 | 0.223027 | 0.037219 | 0.074254 | 0.165361 | 0.02392 |
| b6 | 185delAG − | 0.071645 | 0.082469 | 0.186339 | 0.018543 | 0.054902 | 0.162668 | 0.021838 |
| | BRCA1 + 6174delT − | | | | | | | |
| | BRCA2 | | | | | | | |
| c10 | None | 0.152407 | 0.114467 | 0.334019 | 0.032577 | 0.091569 | 0.285785 | 0.030649 |
| c11 | None | 0.148137 | 0.137834 | 0.367038 | 0.03724 | 0.105697 | 0.305872 | 0.028736 |
| c12 | None | 0.160428 | 0.155071 | 0.356754 | 0.077214 | 0.088941 | 0.403321 | 0.027796 |
| c13 | None | 0.104025 | 0.133231 | 0.277777 | 0.048161 | 0.090496 | 0.231166 | 0.027451 |
| c14 | None | 0.133323 | 0.128782 | 0.317538 | 0.066477 | 0.088205 | 0.294839 | 0.02563 |
| c15 | None | 0.104097 | 0.098892 | 0.233906 | 0.03866 | 0.059912 | 0.124395 | 0.0267 |
| c17 | None | 0.085141 | 0.118503 | 0.264621 | 0.058802 | 0.080716 | 0.302918 | 0.026886 |
| c18 | None | 0.079 | 0.121329 | 0.221749 | 0.046649 | 0.056445 | 0.198746 | 0.020333 |
| c19 | None | 0.141807 | 0.13277 | 0.314689 | 0.065835 | 0.093234 | 0.363493 | 0.027853 |
| c2 | None | 0.096589 | 0.078129 | 0.180491 | 0.044936 | 0.050977 | 0.084144 | 0.028896 |
| c3 | None | 0.143091 | 0.145491 | 0.348444 | 0.045279 | 0.098277 | 0.179742 | 0.022561 |
| c4 | None | 0.139178 | 0.125347 | 0.293209 | 0.060623 | 0.08027 | 0.314689 | 0.023487 |
| c9 | None | 0.198746 | 0.121245 | 0.272627 | 0.03858 | 0.116065 | 0.265724 | 0.03138 |
| c5 | None | 0.173019 | 0.122343 | 0.224533 | 0.048294 | 0.105404 | 0.255784 | 0.027757 |
| c6 | None | 0.184284 | 0.121582 | 0.271495 | 0.048194 | 0.093299 | 0.212274 | 0.026849 |
| c7 | None | 0.198609 | 0.105331 | 0.278163 | 0.047333 | 0.08888 | 0.20547 | 0.038527 |
| c8 | None | 0.180241 | 0.108518 | 0.303128 | 0.051474 | 0.089684 | 0.243164 | 0.03173 |
| c1 | None | 0.168054 | 0.081109 | 0.176165 | 0.040163 | 0.059375 | 0.117522 | 0.025827 |

RQ (Relative quantitation) = 2^{-ΔC}

TABLE 9 (b)
RT-PCR expression values (relative values) for STAT5A, YTHDF3, AUH,
EIF3D, IFI44 and NR4A2 expression values.

| Sample | Mutation | STAT5A | YTHDF3 | AUH | EIF3D | IFI44L | NR4A2 |
|---|---|---|---|---|---|---|---|
| b1 | BRCA1 185delAG | 0.07668 | 0.046552 | 0.007381 | 0.412653 | 0.141121 | 0.010628 |
| b12 | BRCA1 185delAG | 0.036172 | 0.024843 | 0.011328 | 0.205898 | 0.060413 | 0.002545 |
| b13 | BRCA1 185delAG | 0.047301 | 0.035329 | 0.003562 | 0.202922 | 0.036398 | 0.010223 |
| b14 | BRCA1 185delAG | 0.043737 | 0.048935 | 0.010252 | 0.252088 | 0.03688 | 0.004789 |
| b15 | BRCA1 185delAG | 0.03866 | 0.041092 | 0.006138 | 0.187375 | 0.01612 | 0.005617 |
| b2 | BRCA1 185delAG | 0.033749 | 0.022452 | 0.004251 | 0.217487 | 0.153893 | 0.007609 |
| b21 | BRCA1 185delAG | 0.044936 | 0.039582 | 0.005648 | 0.215685 | 0.016805 | 0.005471 |
| b3 | BRCA1 185delAG | 0.051332 | 0.027451 | 0.007134 | 0.28146 | 0.029564 | 0.006634 |
| b9 | BRCA1 185delAG | 0.036398 | 0.040218 | 0.006844 | 0.239318 | 0.027432 | 0.018724 |
| b7 | BRCA1 185delAG | 0.036499 | 0.028776 | 0.004493 | 0.231006 | 0.010055 | 0.012691 |
| b8 | BRCA1 185delAG | 0.051797 | 0.033423 | 0.008315 | 0.302918 | 0.036474 | 0.003815 |
| b16 | BRCA1 5382insC | 0.042512 | 0.057751 | 0.006992 | 0.383687 | 0.10076 | 0.045154 |
| b5 | BRCA1 5382insC | 0.047006 | 0.031907 | 0.010518 | 0.364502 | 0.0395 | 0.007588 |
| b10 | BRCA2 6174delT | 0.033377 | 0.028498 | 0.007224 | 0.184156 | 0.019183 | 0.005941 |
| b11 | BRCA2 6174delT | 0.026942 | 0.029097 | 0.004681 | 0.162781 | 0.074687 | 0.00386 |
| b17 | BRCA2 6174delT | 0.044532 | 0.043015 | 0.011399 | 0.28185 | 0.008663 | 0.012166 |
| b18 | BRCA2 6174delT | 0.046844 | 0.029811 | 0.007184 | 0.268501 | 0.029401 | 0.008315 |
| b19 | BRCA2 6174delT | 0.042218 | 0.034819 | 0.008264 | 0.217789 | 0.011727 | 0.004883 |
| b20 | BRCA2 6174delT | 0.037373 | 0.03386 | 0.007851 | 0.251974 | 0.018155 | 0.011683 |
| b6 | 185delAG − | 0.039941 | 0.027054 | 0.006267 | 0.234718 | 0.052229 | 0.0213 |
| | BRCA1 + 6174delT − | | | | | | |
| | BRCA2 | | | | | | |
| c10 | None | 0.074377 | 0.051653 | 0.012904 | 0.39998 | 0.057075 | 0.004098 |
| c11 | None | 0.082184 | 0.059129 | 0.013555 | 0.437392 | 0.061939 | 0.007129 |
| c12 | None | 0.057472 | 0.056681 | 0.012976 | 0.226723 | 0.093299 | 0.021153 |
| c13 | None | 0.037137 | 0.041926 | 0.01416 | 0.196554 | 0.003543 | 0.008675 |
| c14 | None | 0.054826 | 0.050102 | 0.011711 | 0.252613 | 0.103521 | 0.01655 |
| c15 | None | 0.046391 | 0.0385 | 0.005598 | 0.219 | 0.128514 | 0.015636 |
| c17 | None | 0.042394 | 0.037839 | 0.007489 | 0.253139 | 0.079715 | 0.003879 |
| c18 | None | 0.036983 | 0.030564 | 0.00674 | 0.222365 | 0.009679 | 0.007304 |
| c19 | None | 0.048194 | 0.052995 | 0.011351 | 0.236678 | 0.088695 | 0.01775 |
| c2 | None | 0.040667 | 0.028597 | 0.003175 | 0.191445 | 0.009018 | 0.006529 |
| c3 | None | 0.063153 | 0.041897 | 0.011343 | 0.251913 | 0.155286 | 0.026737 |
| c4 | None | 0.049139 | 0.045123 | 0.011164 | 0.225313 | 0.07315 | 0.018633 |
| c9 | None | 0.061682 | 0.043798 | 0.007641 | 0.226408 | 0.124309 | 0.019791 |
| c5 | None | 0.070024 | 0.045943 | 0.007552 | 0.336342 | 0.051832 | 0.018086 |
| c6 | None | 0.084847 | 0.042365 | 0.008826 | 0.282437 | 0.119908 | 0.021869 |
| c7 | None | 0.064436 | 0.047399 | 0.004826 | 0.339151 | 0.025862 | 0.006583 |
| c8 | None | 0.070707 | 0.046199 | 0.007224 | 0.347239 | 0.221135 | 0.025243 |
| c1 | None | 0.051225 | 0.03776 | 0.003716 | 0.160874 | 0.010316 | 0.005755 |

RQ (Relative quantitation) = 2^{-ΔC}

TABLE 10 (a)
Diagnostic values for MRPS6, CDKN1B, ELF1, NFAT5, NR3C1, SARS and
SMURF2 expression values.

| Sample | Mutation | MRPS6 | CDKN1B | ELF1 | NFAT5 | NR3C1 | SARS | SMURF2 |
|---|---|---|---|---|---|---|---|---|
| b1 | BRCA1 185delAG | 0 | 1 | 1 | 0 | 1 | 1 | 1 |
| b12 | BRCA1 185delAG | 0 | 1 | 0 | 1 | 1 | 1 | 1 |
| b13 | BRCA1 185delAG | 0 | 1 | 1 | 0 | 1 | 1 | 0 |
| b14 | BRCA1 185delAG | 0 | 0 | 0 | 0 | 0 | 0 | 0 |
| b15 | BRCA1 185delAG | 1 | 1 | 0 | 1 | 1 | 1 | 1 |
| b2 | BRCA1 185delAG | 1 | 1 | 1 | 1 | 1 | 1 | 1 |
| b21 | BRCA1 185delAG | 1 | 1 | 1 | 0 | 1 | 1 | 1 |
| b3 | BRCA1 185delAG | 1 | 1 | 1 | 1 | 1 | 0 | 1 |
| b9 | BRCA1 185delAG | 0 | 1 | 1 | 1 | 1 | 1 | 0 |
| b7 | BRCA1 185delAG | 0 | 1 | 1 | 1 | 1 | 1 | 1 |
| b8 | BRCA1 185delAG | 1 | 1 | 0 | 1 | 1 | 0 | 1 |
| b16 | BRCA1 5382insC | 1 | 0 | 0 | 0 | 0 | 1 | 0 |
| b5 | BRCA1 5382insC | 0 | 1 | 1 | 1 | 1 | 0 | 1 |
| b10 | BRCA2 6174delT | 1 | 1 | 1 | 1 | 1 | 1 | 1 |
| b11 | BRCA2 6174delT | 1 | 1 | 1 | 1 | 1 | 1 | 1 |
| b17 | BRCA2 6174delT | 0 | 1 | 0 | 1 | 1 | 1 | 1 |
| b18 | BRCA2 6174delT | 1 | 1 | 1 | 1 | 1 | 1 | 1 |
| b19 | BRCA2 6174delT | 1 | 0 | 0 | 0 | 0 | 0 | 0 |
| b20 | BRCA2 6174delT | 1 | 0 | 1 | 1 | 1 | 1 | 1 |
| b6 | 185delAG − | 1 | 1 | 1 | 1 | 1 | 1 | 1 |
| | BRCA1 + 6174delT − | | | | | | | |
| | BRCA2 | | | | | | | |
| c10 | None | 0 | 0 | 0 | 1 | 0 | 0 | 0 |
| c11 | None | 0 | 0 | 0 | 1 | 0 | 0 | 0 |
| c12 | None | 0 | 0 | 0 | 0 | 0 | 0 | 0 |
| c13 | None | 0 | 0 | 0 | 0 | 0 | 0 | 0 |
| c14 | None | 0 | 0 | 0 | 0 | 0 | 0 | 1 |
| c15 | None | 0 | 1 | 0 | 0 | 1 | 1 | 0 |
| c17 | None | 1 | 0 | 0 | 0 | 0 | 0 | 0 |
| c18 | None | 1 | 0 | 1 | 0 | 1 | 0 | 1 |
| c19 | None | 0 | 0 | 0 | 0 | 0 | 0 | 0 |
| c2 | None | 0 | 1 | 1 | 0 | 1 | 1 | 0 |
| c3 | None | 0 | 0 | 0 | 0 | 0 | 1 | 1 |
| c4 | None | 0 | 0 | 0 | 0 | 0 | 0 | 1 |
| c9 | None | 0 | 0 | 0 | 1 | 0 | 0 | 0 |
| c5 | None | 0 | 0 | 1 | 0 | 0 | 0 | 0 |
| c6 | None | 0 | 0 | 0 | 0 | 0 | 0 | 0 |
| c7 | None | 0 | 1 | 0 | 0 | 0 | 0 | 0 |
| c8 | None | 0 | 0 | 0 | 0 | 0 | 0 | 0 |
| c1 | None | 0 | 1 | 1 | 0 | 1 | 1 | 1 |

0- if greater then cutoff value;
1- if less then cutoff value;

TABLE 10 (b)
Diagnostic values for STAT5A, YTHDF3, AUH, EIF3D, IFI44 and, NR4A2
expression values

| Sample | Mutation | STAT5A | YTHDF3 | AUH | EIF3D | IFI44L | NR4A2 |
|---|---|---|---|---|---|---|---|
| b1 | BRCA1 185delAG | 0 | 0 | 1 | 0 | 0 | 0 |
| b12 | BRCA1 185delAG | 1 | 1 | 0 | 1 | 0 | 1 |
| b13 | BRCA1 185delAG | 0 | 1 | 1 | 1 | 1 | 0 |
| b14 | BRCA1 185delAG | 0 | 0 | 0 | 0 | 1 | 1 |
| b15 | BRCA1 185delAG | 1 | 0 | 1 | 1 | 1 | 1 |
| b2 | BRCA1 185delAG | 1 | 1 | 1 | 1 | 0 | 0 |
| b21 | BRCA1 185delAG | 0 | 0 | 1 | 1 | 1 | 1 |
| b3 | BRCA1 185delAG | 0 | 1 | 1 | 0 | 1 | 1 |
| b9 | BRCA1 185delAG | 1 | 0 | 1 | 0 | 1 | 0 |
| b7 | BRCA1 185delAG | 1 | 1 | 1 | 1 | 1 | 0 |
| b8 | BRCA1 185delAG | 0 | 1 | 0 | 0 | 1 | 1 |
| b16 | BRCA1 5382insC | 0 | 0 | 1 | 0 | 0 | 0 |
| b5 | BRCA1 5382insC | 0 | 1 | 0 | 0 | 1 | 0 |
| b10 | BRCA2 6174delT | 1 | 1 | 1 | 1 | 1 | 1 |
| b11 | BRCA2 6174delT | 1 | 1 | 1 | 1 | 0 | 1 |
| b17 | BRCA2 6174delT | 0 | 0 | 0 | 0 | 1 | 0 |
| b18 | BRCA2 6174delT | 0 | 1 | 1 | 0 | 1 | 0 |
| b19 | BRCA2 6174delT | 1 | 1 | 0 | 1 | 1 | 1 |
| b20 | BRCA2 6174delT | 1 | 1 | 0 | 0 | 1 | 0 |
| b6 | 185delAG − | 1 | 1 | 1 | 1 | 1 | 0 |
| | BRCA1 + 6174delT − | | | | | | |
| | BRCA2 | | | | | | |
| c10 | None | 0 | 0 | 0 | 0 | 0 | 1 |
| c11 | None | 0 | 0 | 0 | 0 | 0 | 1 |
| c12 | None | 0 | 0 | 0 | 1 | 0 | 0 |
| c13 | None | 1 | 0 | 0 | 1 | 1 | 0 |
| c14 | None | 0 | 0 | 0 | 0 | 0 | 0 |
| c15 | None | 0 | 0 | 1 | 1 | 0 | 0 |
| c17 | None | 1 | 1 | 0 | 0 | 0 | 1 |
| c18 | None | 1 | 1 | 1 | 1 | 1 | 0 |
| c19 | None | 0 | 0 | 0 | 0 | 0 | 0 |
| c2 | None | 1 | 1 | 1 | 1 | 1 | 1 |
| c3 | None | 0 | 0 | 0 | 0 | 0 | 0 |
| c4 | None | 0 | 0 | 0 | 1 | 0 | 0 |
| c9 | None | 0 | 0 | 0 | 1 | 0 | 0 |
| c5 | None | 0 | 0 | 0 | 0 | 1 | 0 |
| c6 | None | 0 | 0 | 0 | 0 | 0 | 0 |
| c7 | None | 0 | 0 | 1 | 0 | 1 | 1 |
| c8 | None | 0 | 0 | 1 | 0 | 0 | 0 |
| c1 | None | 0 | 1 | 1 | 1 | 1 | 1 |

0- if greater then cutoff value;
1- if less then cutoff value;

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1-20. (canceled)

21. A composition comprising detecting molecules specific for 6 to twenty marker genes, but containing no more than 384 detecting molecules, wherein said detecting molecules comprise isolated detecting nucleic acid molecules or isolated detecting amino acid molecules and wherein said marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6ORF111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12.

22. The composition according to claim 21, comprising detecting molecules specific for 6 to eighteen marker genes, but containing no more than 384 detecting molecules, wherein said marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6ORF111), splicing factor, arginine/serine-rich 18.

23. The composition according to claim 21, comprising detecting molecules specific for 6 to thirteen marker genes, but containing no more than 384 detecting molecules, wherein said marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2.

24. The composition according to claim 21, wherein said detecting molecules comprise detecting nucleic acid molecules.

25. The composition according to claim 24, wherein said detecting nucleic acid molecules comprise isolated oligonucleotides, wherein each said oligonucleotide specifically hybridizes to a nucleic acid sequence of an RNA product of one of said marker genes, and wherein said oligonucleotides comprise pairs of primers.

26. The composition according to claim 25, wherein said detecting molecules further comprise probes.

27. The composition according to claim 21, wherein said detecting molecules comprise detecting amino acid molecules, said detecting amino acid molecules comprise isolated antibodies, wherein each said antibody binds selectively to a protein product of one of said marker genes.

28. The composition according to claim 21, for the detection of at least one mutation in at least one of BRCA1 and BRCA2 genes in a mammalian subject, wherein a differential expression of said 6 to twenty marker genes as compared to a control population is indicative of at least one mutation in at least one of BRCA1 and BRCA2 genes in said subject, and thereby of an increased genetic predisposition of said subject to a cancerous disorder associated with mutations in any one of BRCA1 and BRCA2 genes.

29. The composition according to claim 21, wherein said detecting molecules are disposed in an array.

30. The composition according to claim 29, wherein said array of detecting molecules comprises a plurality of addressed vessels.

31. The composition according to claim 29 wherein said array of detecting molecules comprises a solid support holding detecting molecules in distinct regions.

32. A diagnostic kit comprising:
(a) detecting molecules specific for 6 to twenty marker genes, but including no more than 384 detecting molecules, wherein said detecting molecules are isolated detecting nucleic acid molecules or isolated detecting amino acid molecules, and wherein said marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2, RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6orf111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12; and
(b) at least one detecting molecule specific for at least one control gene.

33. The kit according to claim 32, wherein said isolated detecting molecules are isolated oligonucleotides, wherein each said oligonucleotide specifically hybridizes to a nucleic acid sequence of an RNA product of one of said marker genes or one of said control genes, and wherein said oligonucleotides comprise pairs of primers.

34. The kit according to claim 33, further comprising at least one reagent for performing a nucleic acid amplification based assay selected from the group consisting of a Real-Time PCR, micro arrays, PCR, in situ Hybridization and Comparative Genomic Hybridization.

35. The kit according to claim 33, wherein said detecting molecules further comprise probes.

36. The kit according to claim 32, wherein each said detecting molecule is an isolated detecting amino acid molecule consisting of an isolated antibody which binds selectively to the protein product of one of said marker genes or said control genes.

37. The kit according to claim 32, for detecting of at least one mutation in at least one of BRCA1 and BRCA2 genes in a mammalian subject, wherein detection of a mutation in any one of BRCA1 or BRCA2 genes is indicative of an increased genetic predisposition of said subject to a cancerous disorder associated with mutations in at least one of BRCA1 and BRCA2.

38. The kit according to claim 32, further including a solid support, wherein each of said detecting molecules is disposed in a distinct region of said solid support.

39. The kit according to claim 32, wherein said detecting molecules specific for marker genes comprise detecting molecules specific for 6 to eighteen marker genes, but containing no more than 384 detecting molecules, wherein said marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; and SFRS18 (C6ORF111), splicing factor, arginine/serine-rich 18.

40. The kit according to claim 32, wherein said detecting molecules specific for marker genes comprise detecting molecules specific for 6 to thirteen marker genes, but containing no more than 384 detecting molecules, wherein said marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; and NR4A2, nuclear receptor subfamily 4, group A, member 2.

41. An array of detecting molecules comprising detecting molecules specific for 6 to twenty marker genes, but containing no more than 384 detecting molecules, wherein said detecting molecules are isolated detecting nucleic acid molecules or isolated detecting amino acid molecules and wherein said marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6ORF111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12.

42. The array of claim 41, comprising a plurality of addressed vessels containing said detecting molecules.

43. The array of claim 41, comprising a solid support holding detecting molecules in distinct regions.

44. A composition comprising detecting molecules specific for 6 to twenty marker genes, wherein said detecting molecules comprise oligonucleotides that are pairs of primers or amino acid molecules that are antibodies, and wherein said marker genes are selected from the group consisting of: MRPS6, mitochondrial ribosomal protein S6; CDKN1B, cyclin-dependent kinase inhibitor 1B (p27, Kip1); ELF1, E74-like factor 1 (ets domain transcription factor); NFAT5, nuclear factor of activated T-cells 5, tonicity-responsive; NR3C1, nuclear receptor subfamily 3, group C, member 1 (glucocorticoid receptor); SARS, seryl-tRNA synthetase; SMURF2, SMAD specific E3 ubiquitin protein ligase 2; STAT5A, signal transducer and activator of transcription 5A; YTHDF3, YTH domain family, member 3; AUH, AU RNA binding protein/enoyl-Coenzyme A hydratase; EIF3D, eukaryotic translation initiation factor 3, subunit D; IFI44L, interferon-induced protein 44-like; NR4A2, nuclear receptor subfamily 4, group A, member 2; RAB3GAP1, RAB3 GTPase activating protein subunit 1 (catalytic); MID1IP1, MID1 interacting protein 1 (gastrulation specific G12 homolog (zebrafish)); RGS16, regulator of G-protein signaling 16; MARCH7, membrane-associated ring finger (C3HC4) 7; SFRS18 (C6ORF111), splicing factor, arginine/serine-rich 18; RPS6KB1, ribosomal protein S6 kinase, 70 kDa, polypeptide 1; and DNAJC12, DnaJ (Hsp40) homolog, subfamily C, member 12.


SEQUENCE LISTING (48 sequences)

SEQ ID NO: 1 - DNA, 4179 bp, Homo sapiens
agcgccaggcccggcgctcctcaagatggctgccgacagtgagcccgaatccgaggtatttgagatcacggacttcaccactgcctcggaatgggaaaggtttatttccaaagttgaagaagtcttgaatgactggaaactgattggaaactctttgggaaagccactcgaaaagggtatatttacttctggcacatgggaagagaaatcagatgaaatttcctttgctgacttcaagttctcagtcactcatcattatcttgtacaagagtccactgataaagaaggaaaggatgagttattagaggatgttgttccacaatctatgcaagatttgctgggtatgaataatgactttcctccaagagcacattgcctggtaagatggtatgggctacgtgagttcgtggtgattgcccctgctgcacacagtgacgctgttctcagcgaatctaagtgcaaccttcttctgagttctgtttctattgccttgggaaacactggctgtcaggtgccactctttgtgcaaattcaccacaaatggcgaagaatgtatgtaggagaatgtcaaggtcctggtgtacgaactgatttcgaaatggttcatcttagaaaagtgccaaatcagtacactcacttatcaggtctgctggatatcttcaaatcaaagattggatgtcctttaactccattgcctccagttagtattgctattcgatttacctatgtacttcaagattggcagcagtatttttggcctcagcaacctccagacatagatgcccttgtaggaggagaagttggaggcttggagtttggcaagttaccatttggtgcctgcgaagatcctattagtgaactccatttagctactacatggcctcatctgaccgaagggatcattgtggataatgatgtttattctgatttggatcctattcaagctccacattggtctgttagagttcgaaaagctgagaatcctcagtgtttgctaggtgattttgtcactgaattttttaaaatttgccgtcgaaaggagtcaactgatgagattcttggacgatctgcatttgaggaagaaggcaaagaaactgctgatataactcatgctttgtcaaaattgacagagccggcatcagttccaattcataaattatcagtttcaaatatggtacacactgcaaagaagaaaatccgaaaacacagaggtgtagaggagtcaccgctaaataatgatgttcttaatactattctcctgttcttattccctgatgctgtttctgagaaaccattagatggaactacttcaacagataataataatcctccatcagagagtgaagactataatctctacaatcagttcaagtctgcaccatctgacagtttaacatacaaactggctttgtgtctctgtatgatcaatttttaccatggagggttgaaaggagtggcacacctctggcaggaatttgttcttgaaatgcgtttccgatgggaaaacaactttctgattccaggattagcaagtggacccccagatctgaggtgttgtttactgcatcagaaactacagatgttaaattgttgtattgaaagaaagaaggcacgtgatgaggggaaaaagacaagtgcttcagatgtcactaatatatatccaggggatgctggaaaagcaggagaccagttggtgccagataatctaaaagaaacagataaggaaaagggagaggtaggaaaatcttgggattcctggagtgacagcgaagaagaattttttgaatgcctaagtgatactgaagaacttaaaggaaatggacaagagagtggcaagaaaggaggacctaaggagatggcaaatttaaggccggaaggacggctctatcagcatgggaaacttacactgctgcataatggagaacctctctacattccagtaacccaggaaccagcacctatgacagaagatctgctagaagagcagtctgaagttttagctaaattaggtacatcggcagagggggctcaccttcgagcacgcatgcagagtgcctgtctgctctcagatatggagtcttttaaggcagctaatccaggttgctccctggaagattttgtgaggtggtattcaccccgggattatattgaagaggaggtgattgatgaaaagggcaatgtggtgctgaaaggagaactgagtgcccggatgaagattccaagcaatatgtgggtagaagcctgggaaacagctaagccaattcctgctagaaggcaaaggagactctttgatgatacacgggaagcagaaaaggtgctgcactatctggcaatccagaaacctgcagaccttgctcggcacctgttaccttgtgtgattcatgcagctgtactcaaggtaaaggaagaagaaagtctcgaaaacatttcttcagttaagaagatcataaagcagataatatcccattccagtaaagttttgcacttccccaatccagaagacaagaaattggaagaaatcattcaccagattactaatgtggaagctctcattgccagagctcggtcactaaaagccaagtttggaactgagaaatgtgaacaggaggaggaaaaggaagatcttgaaaggtttgtgagttgcctgctggagcagcctgaagtgttagtcaccggtgcaggaagaggacatgctggcaggatcattcacaagctgtttgtgaatgcccagagggctgcagctatgactccaccagaggaggaattgaagagaatgggctccccagaggaaagaaggcagaactccgtgtcagacttcccaccccctgctggccgggaattcattttgcgcaccactgtgccgcgccctgctccctactccaaagctctgcctcagcggatgtacagtgttctcaccaaagaggactttagacttgcaggtgccttttcatcagatacttccttcttctgattcttctagcattactcgttggtggcttcagagacagtgctgcctcctcctgagggagggaaggtaccagggagaacctgggaggtcctggagagggccctgtccagttgggtgatcaggaatcaaaccagcatcggaaagacttcccagcaccaagcttgagctgtgtcgtttcgtggagggggcagcgaggatgggcttgagctgttgagagatttctgccctagagatggcctttgtatatgggggggtggtggggggacacaaacacatcagacactccgtcctcacactggcaggacggtgttcatcgcattctcttctgtgaccagcctctaggctagcggctgcattcgtggtctgtgcaaacacttcgtggttctatatatcagcagcaagtgtgcaaaataaaggacctgttaactcagatttctggatattttggtggtagcttctagtcccagaatctgtgtttttaaaatactacatgacattctgtctattcaatcacctggtggtcatctttcttgtactaattaactgttgatgagcattttggatattctaggagaaagcctataatttcacatagtttctctttttcatgtaactgtaacctaaatgtattacttctgataaaactatatatcaaatgtcactgcaaattagttttatatctgtcatgtgagatttgtcttacttatttttcttttggttgccatggaagttatggccctgaaaatcgtctccctccccttctcttgctgtacagcatgcgttctctttttgtggttgctggctgggtactgtatttaatgaagtagagaatagcacttgcaaaaatacagtcttggtacctagagactgtcatgcagatagtataatttggtatatgtgctaatgcattgagtagaggattattttaacacactattttgcttttgtattttagttaaaataatcgatggggatgtgtagcccccccgtgtgaggatgacatcaccacatttctagtttcatggagctcaagatgtcttgtgtctgtgtggctagatggcctctgcttggtaatcttatttttaggcctaaaattcccacttaaatccaaagtaaaaatggttatactgaagcataaaccttgcctgtgtaattttaaaaaattaatagagctgtgcaaaccctgttatttttgtaaaaaaaaaaaaaaa

SEQ ID NO: 2 - DNA, 14063 bp, Homo sapiens
tgcaacggaaacttttggctccacgaacagaaaagcaagagcaaaaactcagttaaatgcgttcttcctccatctttgcctagtccaaaaggaaaaaaaaagaaacagaaaaaagaaaaactgtttggaagagtagcgttgaggtttgctttttacctgtttttcctctagaacagccaggtggattcacatgatccaattttttaattgtcttctatgtcccactccgaataacccggatgccccagcacaatcagagagatcgttttttttaaaaaaaatttcagcctccaaaggtaggaggggagtgttaggggagaaagtactttaattaaaaatcaataactcgaggtttttgggatacggtttgccatttcctaattaagaaatgggtttgacagtccctttgtacacactgctatgcaaaccccaaagggttggcggctgtccgggcgatgacactccggtcccctgcgagaccccgggccagccaggcccgtccgccgccggcctctggggtccgtccccggctcgcgcagacctctcgcttctctcggctctgtctcctgcgctcagctctgctcggggccggccgcctcaggctcgccgccaccaggtcgttgcaaataccttttccctccccggggccccagcgcgcggccacctcccagcctccccccctcccaccctggcagcggggccctttcccggctcaggaacagcagcagcccgggccgcgccggcaggaagcgaggcccatgttgctgctgttccctggcgcgcctccccgccctccgggggccgccacggctcttccgcgctcccgggcacccccctccgcgcctgcgctgtgccccacgggggcggggctcagattcctgtcagcggcggcggcggtggcggcgaccgtcagttttcgctgaggagaaacacgaaacggaccctttggctctcccccttccccttccccgtcctgaacccctctcctggtcaccgagaatcagtccccgtggagttccccctccacctcgccatcgtttcctcggtcctcggcccagtggaagtcactaccctcgaggaggaggcagcggcagccgccctcgcgtcgccgcccccggttcggtgcccgcggtcccggagaggaggtgccgccgccaccgccgctccccccctcccgctgccctcgggccgggctgggtcgagctgcgatgccctcggacttcatctcattgctcagcgcggacctagacctggaatcgcccaagtccctctactcgcgagaatctgtctatgatcttctcccaaaggagttacagttacctccatctagagaaacatctgtagcatcaatgagtcagacaagcggtggtgaggcaggctcgcctcctccagctgttgttgctgctgatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccagtaacacagttcagcagcatccatcaacaccgaagaggcacacagtcttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagaatctaatatggatatatttgatgccgacagtgccaaagcacctcactatgtgctttctcagcttaccacggacaacaaaggcaactcaaaagcgggaaatggaacattggaaaaccaaaaaggaactggagtaaagaagagccctatgttgtgtggacaatatcctgttaaaagtgagggaaaggagctgaagatagttgtacaacctgagacacagcaccgagctcggtacctgactgagggcagccgtggctcagtgaaagatagaacacagcaaggctttcctacagtaaagctggaaggccataatgaacctgtagtgttgcaagtgtttgtgggcaacgactctggacgagtgaaaccacatggattttatcaggcctgcagagtaactggacgaaatacaactccttgcaaagaagtggacattgaaggcactactgttatagaagtcggccttgatcctagcaacaacatgacactggcggtggactgcgtagggatattgaaattgaggaatgctgatgtcgaagccagaataggaattgctggttccaagaagaaaagcactcgtgccagattggtttttcgagttaatatcatgaggaaagatggctccactttgacactgcaaacaccctcttctccaattttgtgtactcagccagcaggagtgccagaaatcttaaagaaaagcttgcatagctgttcagtgaaaggagaagaagaagtgtttttaatcggcaagaactttctgaaaggaactaaagttattttccaagaaaatgtttctgatgaaaactcttggaagtcagaagctgaaattgatatggaactatttcatcagaatcatcttattgtgaaggttcctccctatcatgaccaacatataactttgcctgtgtcagtgggaatatatgtagtgacaaatgctggaagatctcatgatgttcaaccattcacttacactccagacccagcagcagctggtgctttgaatgtaaatgtgaagaaggaaatatctagtccagcaagaccttgctcttttgaagaggccatgaaagcaatgaaaactactggatgtaatttagataaggtaaatattatccctaatgccctgatgactccactcataccaagcagtatgattaagagtgaagatgttactccaatggaagtaacagcagaaaaaagatcttccactatttttaagactacaaagtctgttggatcaactcagcaaacattagaaaacatctcaaacatagcaggaaatggctctttttcatcaccatcatcttcccacctaccttctgaaaatgaaaaacagcagcagattcagcccaaggcatacaacccagagaccctgacaactattcaaacccaggacatctcacagcctggtacttttccagcagtttctgcttctagtcagctgcccaacagcgatgcactattgcagcaggctacacagtttcagacaagagaaactcagtctagagagatattacagtcagatggtacagtggttaatttgtcacaactgactgaggcatcacaacaacagcagcagtcaccactacaagaacaagcacagactttacagcagcagatttcatcaaatatttttccatcaccaaatagtgtgagtcagcttcagaatactattcagcagctgcaagcagggagtttcacaggcagtactgctagtggcagcagtggaagtgttgacttggtccaacaagttttagaggcacagcagcagttatcttcagttttattttctgctccagatggtaatgagaatgttcaagagcagcttagtgcagatatttttcaacaagtcagtcaaattcagagtggtgtaagccctggaatgttttcctcaacagagccaacagtccataccagaccagataatttattacctggaagagctgaaagtgttcatccacagtctgaaaacacgttatctaatcaacagcagcagcagcagcagcaacagcaagtgatggaatcttcagccgcaatggtgatggagatgcaacagagtatctgccaggcagctgcccagattcagtcagagttattcccttcaactgcttcagcaaatggaaaccttcagcaatcgccagtttaccagcagacttctcacatgatgagtgcattgtctaccaatgaggatatgcaaatgcagtgtgaattgttttcttctcctcctgcagtttctggaaatgaaacttctacaactaccacacagcaggttgcaacccctggcactaccatgtttcagacatcaagttcaggagatggagaagaaactggaacacaagcaaaacagattcagaacagtgtctttcagaccatggtccaaatgcaacatagtggggacaatcaacctcaagttaaccttttttcatccacaaaaagtatgatgagtgttcagaatagtggtacccaacaacaaggtaatggtttattccagcaagggaatgagatgatgtcacttcaatctggaaattttttgcagcagtcttctcattcacaggcccaactttttcatcctcaaaatcctattgccgatgctcagaacctttcccaggaaactcaaggttctctctttcatagtccaaatcctattgtccacagtcagacttctacaacctcctctgaacaaatgcagcctccaatgtttcactctcaaagtaccattgctgtgttacagggctcttcagttcctcaagaccagcagtcaaccaacatatttctttcccagagtcccatgaataatcttcagactaacacagtagcccaagaagcattttttgcagcaccgaactcaatttctccacttcagtcaacatcaaacagtgaacaacaagctgctttccaacagcaagctccaatatcacacatccagactcctatgctttcccaagaacaggcacaacccccgcagcagggtttatttcagcctcaggtggccctgggctcccttccacctaatccaatgcctcaaagccaacaaggaaccatgttccagtcacagcactcaatagttgccatgcagagtaactctccatcccaggaacagcagcagcagcagcaacagcagcagcaacagcagcagcaacaacaacagagcattttattcagtaatcagaataccatggctacaatggcgtctccaaagcaaccaccaccaaacatgatattcaacccaaatcaaaatccaatggctaatcaggagcaacagaaccagtcaatttttcaccaacaaagtaacatggccccaatgaatcaagagcaacagcccatgcaatttcagagtcagtccacagtttcctcacttcagaacccaggtcctacccagtcggaatcatcacagacccccttgttccatagctctcctcagattcagttggtacaagggtcacctagttctcaagagcagcaagtaactctcttcttatctccagcatccatgtctgccttgcagaccagtataaatcaacaagatatgcaacagtctcctctttattcccctcagaacaacatgcctggaattcaaggagccacatcttcgcctcaaccacaggctactttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagattgatttgcttgtttcattgcaaaaccaagggaacaacttgactggctccttttaactggatataaattccacgaagaaaatcctgattccaagatgtcctgagatcttgtggttccatgagaattattactttaaaaacaaaacaaaatataaaaaactgtgtttgagtaaactgatagattttactctgactgcaaaagagcacacctatgctgcttgttgcagtaactaaccaccaatgttaacatcttcatattttatattcctaataacagtgatgactgagaatctatttgagtttccagctggcagaattaattgttattattttcctaggcgcaatttccttaaacgtacagtttaaattcaaggctggaccactcagttattattgctattagaaaataatatatcatgtttacttttgttcttcattattttctttcctgcattgttttagtcaagtaatggcttttgaaaaagtaaagttcaataataactaaggctgtgatttttttcaatataaaaggcacagctgttggccaaagtgaaggaatcttttttcagttttattggagaaactgaagggtaacattctaacaagtaaactgtatgtgcagataaaagtactcttgatttaacacaaaggcagatgatacacttataaaactgggaacagctggaatgcttcttgattttattttttcagagagttgttagttctctgggtttctactaaggggtttagccataactgtgcatagaaaaataattatctgtaaaaaatgaaggggataatatatgataaattatgttctgatatcctcctacagtagtttaaattgacagaaaaatttgaatgttttcttcttaacccagtcttaggctggtattccctttttatatatatctatattacttttcacctctttttcactttactttagagaactattaatatactactggcttcatgaccctgtagcatctttggccactttaatctagggtgacctagcaatcctgcagcacagggcagagagtactgtcttaggaattattaggagttgattcctgagaaacaacacatttttccccatgaacggtgctgttctgaagtcttcaaatttttccctctaataggaaacagtataaattttaattaaaaaaaaaaggcaaactaaaatttcttgaaatatcacttctccctgatctgcagtgagtataaattcacttgtcacctcagtgctttacagtttgaagtggtcacttacctgatggttcccacaagccttaggctttacagggttgtatcattgacttaaaatgaagaattaacttgtgttacatctataaagagcaaaataacacactccagaacttggcagttgtagcattagttatacagttttgggtgttcttgccacccgtgggatgcctgcttctcactaccacctgtgtctggacacatgcttatgtctcattttccttttggcatgtggaaagctgtcaatgcagtgtaaggccaacgtgtgtgtggcttctatgtgttgagataatgttttggtatccttgtccgtttcatttattttttaagtgtacaaaaaataacctgttaattgttgaaggctacttttctgttctttttttttttttttttctatcctgtacatttagttgaactgtgcggaattgtggtgttggttttgtttacacagccagatttttccttctttttgttttgtgatgatcttcctttgttctttgaatgtgctcttttgtctttttctcttttttctcatgttttcttccctccacctccacccctttctttctttctctctctgattgagaggcattgaattacgttttcagtagtacaggcttcttgccgatatgaagggaacttttcagaaagagacctactctgggtcatttaattttgaatacagttttcaatcgttcaagttttggatggtttatatctaatgtgtgtttcatttttttggaaagctatattttgtatttaggaaatggtatactattttgctatttgtactgagtgagtacattggcataaatatagaaatttatatatatacatatatataaactattcttttttgccacacatttttgtggtaaatttgtgagtttgtctgatgttctaccacaacgtggcgtctgataacagtgagggggggtggggtttgttatgtctttattgagtatttaagtatcttttgaaacaaatgacctgttcatctgtggccattccatcaggcagttagttccttgatgtcagtagtgggctaaaggcagcttactgtgtgtttgctggagctttcactcagccaagtgttagagtcaggaaacccattgaggcaatggcgtcaaatggtgtttcacaagaatgagccattcagtctttgctcactatatatttaatattttattattgttgttattgttattattaattggctttctgtattctatgccttttatttataaagacactaagaaaacccatgtttgtaattttaataacatttttcccatcttgtaatatccagagctactttataaattctctgaaccaaaagtattttcctcagtgtatctcttctcccccagcccctattgggaaaaattacccagtatagttcaggttatgaggaggatcagccacacaatccagtgcttcagtttgaaaatgtaaaattctaaccctaaagtagggttggttgaaatttcagacaaagcaaacccagcaggtataaaaagtagtataaatacaaatctgtaagttatttttgaattttctgaacttttttctaagagattacataggagactaaagaaatctatctgttcaagttctaattaggatgattgttaatactgcactgtggatgaagtggcgactggcttgtgtgctgacttctgtggtttagcaagaggtttattgttatcaaatgctaattggcaatgccaagtcactgggaccaattttctgttttataatatctaagtttagaacagaatatatacctgaactgtagtggtttgatcggatggagacagaaaacccgatttttattctcataaattttgtggttatttatacaagggctgtgctatgctaccatattcttgttcaataataataggtttgttgttttttttacattgttaaatgttccttacccctaaaggtcaatgttaagtacaacattctgaaaatacaatttggctacgaagagtattcatcttctttgaagctcagtggttgatatttgtgctaataatgcaatttcctgattactgttacaagttatagctacatatgggagagactcagtgagccagcaaaggccatagaaacaacaatttattaaatgtatttatggcagaaggacctaaataaactgtgagccaccttttcttctttatattgttacatttaagtgttcttgctttcagcaactcacattaatgcttggagcttatctctttctctctctctctctctctctctgtgtgtgtgtgtgtatgtgtgtgtgtgtgtgtgtgtgtttccttattgtcattccattatatatccacaccaacatgggtgacgataattcaaagtcatattttgcctctaagcttgatcatgttacctttatgattaaagtatcatgttatttagccaatgcaaatctgttttaaaacaaatagtttaaaaaaagaacaagtttttaagggctttattatagaagaagtattaatgaaggactttccttcctccctccctttcctcccctccctgcctcccttcttcccttccatctccccctcctccctgccttctttgtttctccttcccttattcctccctccctcctttctcccttccttcctttcttccattcatccttccttgccttttatttttattttttgtaatatcacatgtgctgtagtttggaattttattctagtgcatttcttgctcatcagaacctcagctaatctacctaggaaaaatagtatcaaaggaaatgagaaagttgtatctgagtccctccagaactaagataattctttttgaccatttaagcctttataaatgcgttttgaccatttaagcctttataaatgcttgttttaggaaagtgaatctgttagatgcatcaacaaataatgaccaggacaaaacgatttaataattaaagtctcaaatcaccatggttatacattttcaccagaaatagtaatcttacaatttttcatttttctgatgaagatttctgttccaatatctgtttcctaatagattttttaaattaattagctttcctctgctttatgaccacaggttttatccctaaccgagacagctgtcttatatctgcatgccttagactgtgtggagggactccatgaagaaagaccataggttagaaaaataactcatagtatataccctagtaagtgggttagtagaatctcataacatgtatctcataatgcagtaaatatcagaataatatctacaatatcatttgtggatggtcccaggtcccagtgctctagttactttacttctttttttttttttgagatggagtcttgctctgtctctcaggctagagcagtgtgcgatctcagctcactgcagcctccacctcccaggttcaagcgattctcctgcctcagcctcccaagtagccaggattacaggcaccctccactaggcccggctaattttttttgtatttttttagtagagatggggttttgccatgttggccaggctggtttcgaactcctaacctccagtgatccacctgcctcggcgtcccaaagtgctaggattacaggcatgagccaccacatccggcctaattacttctttaatccccatttatttttatgccattctagcctcatttattaataaaattatgtttttactttctctttcaggaaattttttaaattaatattttatatctagatctaatgctatggaaaagtgcctttttatcatttataatttcatttttcactatttccaaaaacacataaacaaatagtttcagtaggtcccagcttttactttttccatttaaaccttcttttctccatttcttccctttggcttaagaataaaagaaaaggtacattgctagaattgtttctttgggagagggtaaaagattacagaattagactgttcagcctttatataaactaaatttgtcttcatctcaaccagctaatggtaggtcttatctgaatactcatgagaattttagcatctgtgaaactccatgcaccagatgtgtgtaaatttcaggaagaaagtgttgaaagcattttctctgatgttaattagatggaaataaatcactaaaacatagtttaggtaaagcctgattatgccacttttttttaactagacagggcaaagttgtttatgttagtgtacttcttgtctatcctcagttaatttacctagacaaaaagtgtcaaaggaaatgagaaaaaggttatatctgactccctccagacctaagataattccttttgatcagatacagtcagatggagtgccttggtttttgttaattttgcctctattccagctccttaccacagcggtggtgcttaaagaaaggatcatcagcaacaggtcaggatagttctacctttgggatagggctgctttccccgtgctagtatttctgtgactgttagtggcactgaggactgcaaacttttatgcaatattcttaataccctattgatattatgcactttaatcattccaaagaagccaagaatgctgtatagtgatgattccttcctaatgaattcatcttaactatttagaatgttatgtcccttttcttttggatagccaacttggtataaatgttatatggatttttctaaaatgactatataggacttaagactttgaaatgtaatttacttataaggggaaataattatgctttagcacatcattttagaaacgtcacattttagaaacattcagcttgctaacctacatgtttgggaattcattaaaaccagttgtctatatattttgtgccatgtatataagaacattacaatatatctttttctacatatgtagtatgtgcaaccagtggttctcagagtatggttctcagcccaccagctagtatcagtatcacctgggaactagttagaaatgtaaattctttggccccatcccagacatactgagtcagaaactctggaatagggcccccgcaatctgttttcacaagccctccaggtgattctgatgcacactttaaagtttaggaaccactgggctaagactctgttgagatatagagtttttcttccactcagactgatatagttatacattgttcttcatgtaaattcagcttaacctggttatctataatcttttattggcaaaagttaattctcagtactgcctatagagatacagtgtattttatgtacatacacaattagtctaattcttgataattcagttaatttagtttggcattttcctaccacttactaaaaggtttacattaaatgactgatttaaatatataggtgcaatgttctatgtttattttaattgttatgacatttaagtagctaatataattgaccggtgctaaagtctcctgtttatccataaaatgggtacattatgggcagtgtaatacaagctttcttttcattgcctagtactttaccagcagaccacagttttgccctggctagaccaaccctcagaacaaaatcatcattccttgtatttatatttgtatctgagatagtaaacaagatggctggccaggtcaacatggcaccttaacttatttttttaataggtaaaacttcttcaaaagtagcttgctttgtataagaactaagctatcagtatagatatagctatccttggagcttatgtttcagacaagaattatttactaaaataaataataaacaagataatgcattatacaatttgggcatttctcgtttctcaagtgtatgcatcatggtaaatataaactaaccacaagataggtagattgattcatttcattttaatctccttgtgtaattcagtacctccataattgttctaatcttcttcccactgtttacaaattaccagttaattaactcgtgaaagaaaaattcacatatcagaataaaaataaatgtatactcactttataaaaatcaccactgctgtctttccttaatactagcagtggaaatgtaagtggcttactctacaaattttggtgctggcaaatacataggcaaactgttgggagctgctctagttacattcctcccttcttattccctttttctcttcctcactttattgcataacatattcctgtacccaaagcattctaccacagttctatttgactcccacttgtaataactcctttaaaaaattccatgtttaaccatatgaccctgcttgcttactcatattctccctccctctccccttcctttctctctcttccagaagtcatttgcctggtttgaaatattttgtagggattgcttattatattattttagctgatgaacctcaggacaacgtctacacacacacacatacatacacgcacacaaaatctcagctgttgaagagtgggcttggaatcagacttctgtgtccagtaaaaaactcctgcactgaagtcattgtgacttgagtagttacagactgattccagtgaacttgatctaatttcttttgatctaatgaatgtgtctgcttaccttgtttccttttaattgataagctccaagtagttgctaattttttgacaactttaaatgagtttcattcacttcttttacttaatgttttaagtatagtaccaataatttcattaacctgttctcaagtggtttagctaccattctgccatttttaatttttatttaattttatttgcttgagcacactgatcaaccactgaactgccttcttccattgtcctgcaatgatataagggttacatttttgtgtatatggctttcatagttgggatttcagagcactgataccagatattttcagtttgttctctgggggaatttcatttgcatctatgtttttagctatctgtgataacttgttaaatattaaaaagatattttgcttctattggaacatttgtatactcgcaactatatttctgtaaacagctgcagtcaaaaataaaacactgaaagttaaaaaaaaaaaaaaaaaaaa

SEQ ID NO: 3 - DNA, 1017 bp, Homo sapiens
cggcgtctgcgcagctgccagcgcctttaagcccgggctcgcgctctcggaccgtgctttcgccgcctgggagccgtccggcgcagcagtttctaggtccccactgtccccgccgtcccgccccttcgcgtcccgggaaccggctggcttccgagccgcactcgccgatcctccaggcatgccccgctacgagctggctttaatcctgaaagccatgcagcggccagagactgctgctactttgaaacgtacgatagaggccctgatggacagaggagcaatagtgagggacttggaaaacctgggtgaacgagcgcttccttataggatctctgcccacagtcagcagcacaacagaggcgggtatttcttggtggatttttatgcacccaccgcagctgttgaaagcatggtggagcacttgtctcgagatatagatgtgattagagggaatattgtcaaacaccctctgacccaggaactaaaagaatgtgaagggattgtcccagtcccactcgcagaaaaattatattccacaaagaagaggaagaagtgagaagattcgccagattttagccttatatgtaattccttcacatttgggcagcatggacgagaaggaagaatttgcaagtttggcctttatataagcatgtgttgcaggtgctgtttgatttttctaaggtatttttagcccttgatcccctttgcttgcgagaggtggggaactgctcactgacagcttctctgtaacctgcagtaccagtggatcgttcttgattttgttttcattagtgtcatttctttgtcattgaggacttttccccttacaacagtaacaccattttttgaagagcaaaacttataatacctcctgggattgtgagctagtcattcagcctgtgtaaccatgtggaaataaaaattgacgaccaatgtattatatggacaacttttgctttgagtaataaacttgattgtaggaatgtgaaaaaaaaaaaaaaaaaaaaaaaaaaa

SEQ ID NO: 4 - DNA, 1601 bp, Homo sapiens
gcggccgtcgcaggtccacgccgtaaacagacaacatggcggccgcggtggcggcggcacctggggccttgggatccctgcatgctggcggcgcccgcctggtggccgcttgcagtgcgtggctctgcccggggttgaggctgcccggctcgttggcaggccggcgagcgggcccggcgatctgggcccagggctgggtacctgcggccgggggtcccgccccgaaaaggggctacagctctgagatgaagacggaggacgagctgcgggtgcggcacctggaggaggagaaccgaggaattgtggtgcttggaataaacagagcttatggcaaaaattcactcagtaaaaatcttataaaaatgctatcaaaagctgtggatgctttgaaatctgataagaaagtacggaccataataatcaggagtgaagtcccagggatattctgtgctggtgctgaccttaaggaaagagccaaaatgagttccagtgaagttggtccttttgtctccaaaataagagcagtgattaacgatattgctaatcttccagtaccaacaattgcagcaatagatggactcgctttaggtggtggtcttgaactggctttagcctgtgatatacgagtagcagcttcctctgcaaaaatgggcctggttgaaacaaaattggcgattattcctggtggaggggggacacagcgattgccacgcgccattggaatgtccctggccaaggagctcatattctctgcgcgagtcctcgatggcaaagaagccaaagcagtgggcttaatcagccacgttctggaacagaaccaggagggagacgcggcctacaggaaggccttggacctggcgagagagtttttacctcagggacctgttgcaatgagagtggcaaaattagcaattaatcaagggatggaggtcgatttagtaacagggttagccatagaagaagcttgttatgctcagaccattccaacaaaagacagacttgaaggtcttcttgcttttaaagagaaaaggccccctcgctataaaggagaataaaaggaacagaaattcttaagatgccaatgtaataaatgtacttcctggaagtgtctttcggatccactatatgcctcagcacatggaaccttaatgaccaaagtgaagagcagattattcatacggtgtaataagcatctggaatggacccatccgtgtacttcattcaaatgtgtaaatgtcatattcattcagatttataaagctagtagtgtatagtcagaaacagaatcaaagttagatatacatttttaaatatttactgcatatgaggctttctgttaattttttaatgtgaataatttatatattgcacattctagggaataatattgattgtatgtctactgtgctgcattaagaaaataaaatttctatataccaaaaatgtgaagttataccaaataaagtttctaagtgattaatgcatacgaacagctacatatacatatatctaaacctgaaaaatgaattgatattctgagtgaaaactacctaatataaataaaattagtgaaaagaaaacatgggaaaaaaaaaaaaaaa

SEQ ID NO: 5 - DNA, 2454 bp, Homo sapiens
ggctcactctgcaaccaaggcacgtgcattctggtcatcccacgcggggagcgcgcgcaaggcccgcccagcccccacatgccagccccaccctccagtcggtccggacgccgacgcctttttgaccctcgctgtgcccggccctcctcatctggcctgcccagggcttggtgctggcggggtccagctgctccaatccctcctcctctgctctgccctgccctgccctggcctgccccggcgccctccctcagcccgggtatcaggcgagaggcggagctggcccggcgcgccccgcccccgctgtagaaagggccgggcgagtgttactcgcggtcatcccggcctgggccttttatctcggtgctgccgggggaggcgggaggaggagacaccaggggtggccctgagcgccggcgacacctttcctggactataaattgagcacctgggatgggtagggggccaacgcagtcaccgccgtccgcagtcacagtccagccactgaccgcagcagcgcccttgcgtagcagccgcttgcagcgagaacactgaattgccaacgagcaggagagtctcaaggcgcaagaggaggccagggctcgacccacagagcaccctcagccatcgcgagtttccgggcgccaaagccaggagaagccgcccatcccgcagggccggtctgccagcgagacgagagttggcgagggcggaggagtgccgggaatcccgccacaccggctatagccaggcccccagcgcgggccttggagagcgcgtgaaggcgggcatccccttgacccggccgaccatccccgtgcccctgcgtccctgcgctccaacgtccgcgcggccaccatgatgcaaatctgcgacacctacaaccagaagcactcgctctttaacgccatgaatcgcttcattggcgccgtgaacaacatggaccagacggtgatggtgcccagcttgctgcgcgacgtgcccctggctgaccccgggttagacaacgatgttggcgtggaggtaggcggcagtggcggctgcctggaggagcgcacgcccccagtccccgactcgggaagcgccaatggcagctttttcgcgccctctcgggacatgtacagccactacgtgcttctcaagtccatccgcaacgacatcgagtggggggtcctgcaccagccgcctccaccggctgggagcgaggagggcagtgcctggaagtccaaggacatcctggtggacctgggccacttggagggtgcggacgccggcgaagaagacctggaacagcagttccactaccacctgcgcgggctgcacactgtgctctcgaaactcacgcgcaaagccaacatcctcactaacagatacaagcaggagatcggcttcggcaattggggccactgaggcgtggcgcccgtggctgcccagcaccttcttcgacccatctcaccctctctcattcctcaaagctttttttttttttcctggctggggggcgggaagggcagactgcaaactggggggctgcgtacgtgcaggaggcgcggtggggctgcgtggaggagggggccacgtgtgagagagaagaaaatggtggccggagatgggagggcccaaggaacctcctgggagggggcctgcattctatgttggtgggaatgggactgggctgacgccctgcattcagcctgtgcctttcctggggtttcttttctgttcttttcggaggagagggcccgagaaggggccataccagggcgcggcgctgggttgccacacttgggaaagcagcccggagctgggtgctggggaaggcggggcgcgtagcctcccgccgccctgcggttgggccggtggaggcccaggcgttgctaggattgcatcagttttcctgtttgcactatttctttttgtaacattggccctgtgtgaagtatttcgaatctcctccttgctctgaaacttcagcgattccattgtgataagcgcacaaacagcactgtctgtcggtaatcggtactactttattaatgattttctgttacactgtatagtagtcctatggcacccccaccccatccctttcgtgccactcccgtccccacccccaccccagtgtgtataagctggcatttcgccagcttgtacgtagcttgccactcagtgaaaataataacattattatgagaaagtggacttaaccgaaatggaaccaactgacattctatcgtgttgtacatagaatgatgaagggttccactgttgttgtatgtcttaaatttatttaaaactttttttaatccagatgtagactatattctaaaaaataaaaaagcaaatgtgtcaactaaattggacaagcgtctggtcctcattaatctgccaatgaatggtttcgtcattaaataaaaatcaatttaattgatttactagcaaaaaaaaaaaaaa

SEQ ID NO: 6 - DNA, 2432 bp, Homo sapiens
aagcagcggccgctcagtctgggcgcttgcaggctgctaaacccaaccgcagttgactagcacctgctaccgcgcctttgcttcctggcgcacgcggagcctcctggagcctgccaccatcctgcctactacgtgctgccctgcgcccgcagccatgtgccgcaccctggccgccttccccaccacctgcctggagagagccaaagagttcaagacacgtctggggatctttcttcacaaatcagagctgggctgcgatactgggagtactggcaagttcgagtggggcagtaaacacagcaaagagaatagaaacttctcagaagatgtgctggggtggagagagtcgttcgacctgctgctgagcagtaaaaatggagtggctgccttccacgctttcctgaagacagagttcagtgaggagaacctggagttctggctggcctgtgaggagttcaagaagatccgatcagctaccaagctggcctccagggcacaccagatctttgaggagttcatttgcagtgaggcccctaaagaggtcaacattgaccatgagacccacgagctgacgaggatgaacctgcagactgccacagccacatgctttgatgcggctcaggggaagacacgtaccctgatggagaaggactcctacccacgcttcctgaagtcgcctgcttaccgggacctggctgcccaagcctcagccgcctctgccactctgtccagctgcagcctggacgagccctcacacacctgagtctccacggcagtgaggaagccagccgggaagagaggttgagtcacccatccccgaggtggctgcccctgtgtgggaggcaggttctgcaaagcaagtgcaagaggacaaaaaaaaaaaaaaaaaaaaaaaaaatgcgctccagcagcctgtttgggaagcagcagtctctccttcagatactgtgggactcatgctggagaggagccgcccacttccaggacctgtgaataagggctaatgatgagggttggtggggctctctgtggggcaaaaaggtggtatgggggttagcactggctctcgttctcaccggagaaggaagtgttctagtgtggtttaggaaacatgtggataaagggaaccatgaaaatgagaggaggaaagacatccagatcagctgttttgcctgttgctcagttgactctgattgcatcctgttttcctaattcccagactgttctgggcacggaagggaccctggatgtggagtcttcccctttggccctcctcactggcctctgggctagcccagagtcccttagcttgtacctcgtaacactcctgtgtgtctgtccagccttgcagtcatgtcaaggccagcaagctgatgtgactcttgccccatgcgagatatttatacctcaaacactggcctgtgagccctttccaagtcagtggagagccctgaaaggaggctcacttgaatccagctcagtgctctgggtggccccctgcaggtggcccctgaccctgcgttgcagcagggtccacctgtgagcaggcccgccctggggcctcttcctggatgtgccctctctgagttctgtgctgtctcttggaggcagggcccaggagaacaaagtgtggaggcctcggggagtggcttttccagctctcatgccccgcagtgtggaacaaggcagaaaaggatcctaggaaataagtctcttggcggtccctgagagtcctgctgaaatccagccagtgttttttgtggtatgagaacaggcaaaaagagatgccccgagatagaaggggagccttgtgtttctttcctgcagacgtgagatgaacactggagtgggcagaggtggcccaggaccatggcacccttagagtgcagaagctggggggagaggctgcttcgaagggcaggactggggataatcagaacctgcctgtcacctcagggcatcactgaacaaacatttcctgatggcaactcctgcggcagagcccaggctggggaagtgaactacccagggcagcccctttgtggcccaggataatcaacactgttctctctgtaccatgagctcctccaggagattatttaagtgtattgtatcattggttttctgtgattgtcataacattgtttttgttattgttggtgctgttgttatttattattgtaatttcagtttgcctctactggagaatctcagcaggggtttcagcctgactgtctccctttctctaccagactctacctctgaatgtgctgggaacctcttggagcctgtcaggaactcctcactgtttaaatatttatttattgtgacaaatggagctggtttcctagatatgaatgatgtttgcaatccccattttcctgtttcagcatgttatattcttataaaataaaagcaaaagtcaaatatga

SEQ ID NO: 7 - DNA, 3484 bp, Homo sapiens
ggtggctggttctgcgccggatccgggagaggggcgggcgccattgtgcttcgctgccgactgcatttcctcagtcacgggcctagaactccaaggagaaaggcggcgaaaaatctttaagaatggagtctaaaccttcaaggattccaagaagaatttctgttcaaccttccagctccttaagtgctaggatgatgtctggaagcagaggaagtagtttaaatgatacctatcactcaagagactcttcatttagattggattctgaatatcagtctacatcagcatcagcatctgcgtcaccatttcaatctgcatggtatagtgaatctgagataactcagggagcacgctcaagatcgcagaaccagcaacgggatcatgattcaaaaagacctaaactttcctgtacaaactgtactacctcagctgggagaaatgttggaaatggtttaaacacattatcagattcatcttggaggcatagtcaagttcctagatcttcatcaatggtacttggatcatttggaacagacttaatgagagagaggagagatttggagagaagaacagattcctctattagtaatcttatggattatagtcaccgaagtggtgatttcacaacttcatcatatgttcaagacagagttccttcatattcacaaggagcaagaccaaaagaaaactcaatgagcactttacagttgaatacatcatccacaaaccaccaattgccttctgaacatcagaccatactaagttctagggactccagaaattctttaagatcaaatttttcttcaagagaatcagaatcttcccgaagcaatacgcagcctggattttcttacagttcaagtagagatgaagccccaatcataagcaattcagaaagggttgtttcatctcaaagaccatttcaagaatcttctgacaatgaaggtaggcggacaacgaggagattgctgtcacgcatagcttctagcatgtcatctacttttttttcacgaagatctagtcaggattccttgaatacaagatcattgaattctgaaaattcttacgtttctccaagaatcttgacagcttcacagtcccgtagtaatgtaccatcagcttctgaagttcccgataatagggcatctgaagcttctcagggatttcgatttcttaggcgaagatggggtttgtcatctcttagccacaatcatagctctgagtcagattcagaaaattttaaccaagaatctgaaggtagaaatacaggaccatggttatcttcctcacttagaaatagatgcacacctttgttctctagaaggaggcgagagggaagagatgaatcttcaaggatacctacctctgatacatcatctagatctcatatttttagaagagaatcaaatgaagtggttcaccttgaagcacagaatgatcctcttggagctgctgccaacagaccacaagcatctgcagcatcaagcagtgccacaacaggtggctctacatcagattcggctcaaggtggaagaaatacaggaatatcagggattcttcctggttccttattccggtttgcagtccctccagcacttgggagtaatttgaccgacaatgtcatgatcacagtagatattattccttcaggttggaattcagctgatggtaaaagtgataaaactaaaagtgcgccttcaagagatccagaaagattgcagaaaataaaagagagcctccttttagaggactcagaagaagaagaaggtgacttatgtagaatttgtcaaatggcagctgcatcatcatctaatttgctgatagagccatgcaagtgcacaggaagtttgcagtatgtccaccaagactgtatgaaaaagtggttacaggccaaaattaactctggttcttcattagaagctgtaaccacctgtgaactatgtaaagagaagttggagcttaacctggaggattttgatattcatgaactacatagagctcatgcaaatgaacaagctgagtatgagtttatcagctctggtctctacctagtggtgttattgcacttgtgcgaacaaagcttttctgatatgatgggaaatacaaatgaaccaagcacacgtgtccgatttattaaccttgcaagaactcttcaggcacatatggaagatctcgaaacttcagaggatgattccgaagaagacggagaccataacaggacatttgatattgcctaacttcatataagacagatggatgatctgtgaacataagtgtttattaaaaatggcaattaaatataaattacttttgtgggggaatgcctaataaatacattgactatatataaaatgaatatatacatacacatgtatgcctgtatatatatattcattctccagtgttgctgaattaaaattctgctggactttttaacatagcaaatccgatgtttataaactggtaatcaaaaaggttttttcttttaggtgagtgggaaagtattacccttgttttaaatatctaagcaatgcctatcaacccttttttgtgttatgattactgtagtcatatttatgaaaaaaggtttgtgttttactcttgctagtgagaaaagtgggacaaaatatacttttgaaataaaatgctatatggcacctaattattttttcttttaaaatgccttaagttgcagtctcattttgataatcatttgcttccagtgtttaaaaattaaaaaaagaatggggagaaggttatgagaagagcattattaagtttccaaatttaatttgaattccaaattcacctagcaataaaatctaatttttaaaaagtatataaatataaaatgtataaatgatggatagatttttgtattgatttgcaaaatgcagattatatttgataggctatagtatgtagatattccttttaggaatattacagctgtaaattatatgagacttgccagtcaaatgctatttggtttaaaaaaattattgcaatctcaagttaatggaatatttttaaatcccacattcagagtttaaaacactggttttcaatgtgttttttagtgttgtcacttgtttatagataaatatataaataacctgtttggatcctggtcctttttaactgttccttggtaattctgagcatttatttgatgacttaatatttttcactacctttggagaacagatgaacattattcaccatgaatggatctatactgtgtggtcatgagttgtgtatacttccataacactgtatttttcttctgtcagtacccttaggatacactttaaaacaccttaaggtctgatgttatggcaacaaactactttttcaaacctaaataggaaccatgtaatttctcaaaagtgattgaacagtttgcccacacttagtttgttggtcttatgtaaaacattggctcaaaataaagtacacactgatttaaaaaaaaaaaaaaaaa

SEQ ID NO: 8 - DNA, 3791 bp, Homo sapiens
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaaaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgcttttttc

SEQ ID NO: 9 - DNA, 3499 bp, Homo sapiens
gccaagaagcttgagagaagaaaaatttcagaaaaattgtctcaatttgactagaatatcaatgaaccaggaaaactgaagcaccttccctaaagaaaacttgggtatacaattactccacagacagagctgagggttttttacccaaatcagtcactggattttgctgcctgatacgtgaatcttcttggaatttttctcatgtggatctaaggggaatgctttattatggctgctgttgtccaacagaacgacctagtatttgaatttgctagtaacgtcatggaggatgaacgacagcttggtgatccagctatttttcctgccgtaattgtggaacatgttcctggtgctgatattctcaatagttatgccggtctagcctgtgtggaagagcccaatgacatgattactgagagttcactggatgttgctgaagaagaaatcatagacgatgatgatgatgacatcacccttacagttgaagcttcttgtcatgacggggatgaaacaattgaaactattgaggctgctgaggcactcctcaatatggattcccctggccctatgctggatgaaaaacgaataaataataatatatttagttcacctgaagatgacatggttgttgccccagtcacccatgtgtccgtcacattagatgggattcctgaagtgatggaaacacagcaggtgcaagaaaaatatgcagactcaccgggagcctcatcaccagaacagcctaagaggaaaaaaggaagaaaaactaaaccaccacgaccagattccccagccactacgccaaatatatctgtgaagaagaaaaacaaagatggaaagggaaacacaatttatctttgggagtttttactggcactgctccaggacaaggctacttgtcctaaatacatcaagtggacccagcgagagaaaggcatttttaaattggtggattctaaagcagtgtccaggttgtgggggaagcacaaaaacaaacctgatatgaattatgagaccatgggaagagcactcaggtactattaccaaaggggtattctggcaaaagtggaaggtcagcgcttggtgtatcagtttaaagaaatgccaaaagatcttatatatataaatgatgaggatccaagttccagcatagagtcttcagatccatcactatcttcatcagccacttcaaataggaatcaaaccagccggtcgagagtatcttcaagtccaggggtaaaaggaggagccactacagttctaaaaccagggaattctaaagctgcaaaacccaaagatcctgtggaagttgcacaaccatcagaagttttgaggacagtgcagcccacgcagtctccatatcctacccagctcttccggactgttcatgtagtacagccagtacaggctgtcccagagggagaagcagctagaaccagtaccatgcaggatgaaacattaaattcttccgttcagagtattaggactatacaggctccaacccaagttccagtggttgtgtctcctaggaatcagcagttgcatacagtaacactccaaacagtgccactcacaacagttatagccagcacagatccatcagcaggtactggatctcagaagtttattttacaagccattccatcatcacagcccatgacagtactgaaagaaaatgtcatgctgcagtcacaaaaggcgggctctcctccttcaattgtcttgggccctgcccaggttcagcaggtccttactagcaatgttcagaccatttgcaatggaaccgtcagtgtggcttcctctccatccttcagtgctactgcacctgtggtgaccttttctcctcgcagttcacagctggttgctcacccacctggcactgtaatcacttcagttatcaaaactcaagaaacaaaaactcttacacaggaagtagagaaaaaggaatctgaagatcatttgaaagagaacactgagaaaacggagcagcagccacagccttatgtgatggtagtgtccagttccaatggatttacttctcaggtagctatgaaacaaaacgaactgctggaacccaactctttttagttaatataccaaagcttatgaataattgtttgttaattgaacattttcaattatatgcagactgactgattctaagataaattctaaggaggtttctaattttgtaattgttaaaaatagagttaattttgactttgttagatgagggaggaaaactcaactgtttctctttgttatctaaatgtttcagaattcaatcgtgaaggaacaggcattttacactatgaagacattcttttgagatttttatttcagttgctatatcataagcatttttaaagtttcttttctaattttacattgtattagattttctgattcttttgtaaatacagaacttaaatagaaggcaacaggaaatttatataggaactattttcattccacttgtgtaagttaagtcttgactctttcaaatgcaaaaaacctattttatgctttgttaaaattatggtgtcacttagattgactttagttgactgcactatataatatagaactatgaatatgtagaataacatgaaaaattggaggtgctggtggtatggctgaccctgtttcagaagcaggatagtataaaagcatcagcctaagaatggcactcccactaactagctatgtaatcttgacctctttgggctttagttcctctcataaaaggaagagatgtattggattagactagattatcaccactttctcttctagttctaatttttttaattctaatacctatattttcaagttatgtcaattaaatcattatcaggttatttcctaatgtaagaatagctaaaatgttgcagagaaataagtgacccaacaaaatttattcatctgttatgggtaagatctgccataaattcttcctaaataatttgtttactaactctttaggccactgtgctttgcggtccattagtaaacttgtgttgctaagtgctaaacagaatactgctattttgagagagtcaagactctttcttaagggccaagaaagcaacttgagccttgggctaatctggctgagtagtcagttataaaagcataattgctttatattttggatcattttttactgggggcggacttggggggggttgcatacaaagataacatatatatccaactttctgaaatgaaatgtttttagattactttttcaactgtaaataatgtacatttaatgtcacaagaaaaaaatgtcttctgcaaattttctagtataacagaaatttttgtagatgaaaaaaatcattatgtttagaggtctaatgctatgttttcatattacagagtgaatttgtatttaaacaaaaatttaaattttggaatcctctaaacatttttgtatctttaattggtttattattaaataaatcatataaaaattctcaaaaaaaaaaaaaaa

SEQ ID NO: 10 - DNA, 5332 bp, Homo sapiens
gctgaactttaggagccagtctaaggcctaggcgcagacgcactgagcctaagcagccggtgatggcggcagcggctgtggtggctgcggcgggtccgggcccatgaggcgacgaaggaggcgggacggcttttacccagccccggacttccgagacagggaagctgaggacatggcaggagtgtttgacatagacctggaccagccagaggacgcgggctctgaggatgagctggaggaggggggtcagttaaatgaaagcatggaccatgggggagttggaccatatgaacttggcatggaacattgtgagaaatttgaaatctcagaaactagtgtgaacagagggccagaaaaaatcagaccagaatgttttgagctacttcgggtacttggtaaagggggctatggaaaggtttttcaagtacgaaaagtaacaggagcaaatactgggaaaatatttgccatgaaggtgcttaaaaaggcaatgatagtaagaaatgctaaagatacagctcatacaaaagcagaacggaatattctggaggaagtaaagcatcccttcatcgtggatttaatttatgcctttcagactggtggaaaactctacctcatccttgagtatctcagtggaggagaactatttatgcagttagaaagagagggaatatttatggaagacactgcctgcttttacttggcagaaatctccatggctttggggcatttacatcaaaaggggatcatctacagagacctgaagccggagaatatcatgcttaatcaccaaggtcatgtgaaactaacagactttggactatgcaaagaatctattcatgatggaacagtcacacacacattttgtggaacaatagaatacatggcccctgaaatcttgatgagaagtggccacaatcgtgctgtggattggtggagtttgggagcattaatgtatgacatgctgactggagcacccccattcactggggagaatagaaagaaaacaattgacaaaatcctcaaatgtaaactcaatttgcctccctacctcacacaagaagccagagatctgcttaaaaagctgctgaaaagaaatgctgcttctcgtctgggagctggtcctggggacgctggagaagttcaagctcatccattctttagacacattaactgggaagaacttctggctcgaaaggtggagcccccctttaaacctctgttgcaatctgaagaggatgtaagtcagtttgattccaagtttacacgtcagacacctgtcgacagcccagatgactcaactctcagtgaaagtgccaatcaggtctttctgggttttacatatgtggctccatctgtacttgaaagtgtgaaagaaaagttttcctttgaaccaaaaatccgatcacctcgaagatttattggcagcccacgaacacctgtcagcccagtcaaattttctcctggggatttctggggaagaggtgcttcggccagcacagcaaatcctcagacacctgtggaatacccaatggaaacaagtggcatagagcagatggatgtgacaatgagtggggaagcatcggcaccacttccaatacgacagccgaactctgggccatacaaaaaacaagcttttcccatgatctccaaacggccagagcacctgcgtatgaatctatgacagagcaatgcttttaatgaatttaaggcaaaaaaggtggagagggagatgtgtgagcatcctgcaaggtgaaacgactcaaaatgacagtttcagagagtcaatgtcattacatagaacacttcagacacaggaaaaataaacgtggattttaaaaaatcaatcaatggtgcaaaaaaaaacttaaagcaaaatagtattgctgaactcttaggcacatcaattaattgattcctcgcgacatcttctcaaccttatcaaggattttcatgttgatgactcgaaactgacagtattaagggtaggatgttgcttctgaatcactgttgagttctgattgtgttgaagaagggttatcctttcattaggcaaagtacaaaattgcctataatacttgcaactaaggacaaattagcatgcaagcttggtcaaactttttccagcaaaatggaagcaaagacaaaagaaacttaccaattgatgttttacgtgcaaacaacctgaatcttttttttatataaatatatatttttcaaatagatttttgattcagctcattatgaaaaacatcccaaactttaaaatgcgaaattattggttggtgtgaagaaagccagacaacttctgtttcttctcttggtgaaataataaaatgcaaatgaatcattgttaaccacagctgtggctcgtttgagggattggggtggacctggggtttattttcagtaacccagctgcaatacctgtctgtaatatgagaaaaaaaaaatgaatctatttaatcatttctacttgcagtactgctatgtgctaagcttaactggaagccttggaatgggcataagttgtatgtcctacatttcatcattgtcccgggcctgcattgcactggaaaaaaaaatcgccacctgttcttacaccagtatttggttcaagacaccaaatgtcttcagcccatggctgaagaacaacagaagagagtcaggataaaaaatacatactgtggtcggcaaggtgagggagatagggatatccaggggaagagggtgttgctgtggcccactctctgtctaatctctttacagcaaattggtaagattttcagttttacttctttctactgtttctgctgtctaccttccttatatttttttcctcaacagttttaaaaagaaaaaaaggtctatttttttttctcctatacttgggctacattttttgattgtaaaaatatttgatggccttttgatgaatgtcttccacagtaaagaaaacttagtggcttaatttaggaaacatgttaacaggacactatgtttttgaaattgtaacaaaatctacataaatgatttacaggttaaaagaataaaaataaaggtaactttacctttcttaaatatttcctgccttaaagagagcatttccatgactttagctggtgaaagggtttaatatctgcagagctttataaaaatatatttcagtgcatactggtataatagatgatcatgcagttgcagttgagttgtatcaccttttttgtttgtcttttataatgtcttcagtctgagtgtgcaaagtcaatttgtaatattttgcaaccctaggatttttttaaatagatgctgcttgctatgttttcaaacctttttgagccataggatccaagccataaaattctttatgcatgttgaattcagtcagaaaagagcaaggctttgctttttgaaattgcaactcaaatgagatgggatgaaatcctatgacagtaagcaaaaacagaaccatgaaaaatgattggacatacaccttttcaattgtggcaataattgaaagaatcgataaaagttcatctttggacagaaagcctttaaaaaaaaaatcactccctcttccccctcctcccttattgcagcagcctactgagaactttgactgttgctggtaaattagaagctacaataataattaagggcagaaattatacttaaaaagtgcagatccttgttctttgacaatttgtgatgtctgaaaaaacagaacccgaaaagctatggtgatatgtacaggcattatttcagactgtaaatggcttgtgatactcttgatacttgttttcaaatatgtttactaactgtagtgttgactgcctgaccaaattccagtgaaacttatacaccaaaatattcttcctaggtcctatttgctagtaacatgagcactgtgattggctggctataaccaccccagttaaaccattttcataattagtagtgccagcaatagtggcaaacactgcaacttttctgcataaaaagcattaattgcacagctaccatccacacaaatacatagtttttctgacttcacatttattaagtgaaatttatttcccatgctgtggaaagtttattgagaacttgtttcataaatggatatccctactatgactgtgaaaacatgtcaagtgtcacattagtgtcacagacagaaagcacacacctatgcaatatggcttatctatatttatttgtaaaaatccaagcatagtttaaaatatgatgtcgatattactagtcttgagtttctaagagggttctttatgttataccaggtaagtgtataaaagagattaagtgcttttttttcatcacttgattattttctttaaaatcagctattacaggatatttttttattttatacatgctgttttttaattaaaatataatcactgaagtttactaatttgattttataaggtttgtagcattacagaataactaaactgggatttataaaccagctgtgattaacaatgtaaagtattaattattgaactttgaaccagatttttaggaaaattatgttctttttccccctttatggtcttaactaatttgaatccttcaagaaggatttttccatactattttttaagatagaagataatttgtgggcaggggtggaggatgcatgtatgatactccataaattcaacattctttactataggtaatgaatgattataaacaagatgcatcttagatagtattaatatactgagccttggattatatatttaatataggacctattttgaatattcagttaatcatatggttcctagcttacaagggctagatctaagattattcccatgagaaatgttgaatttatgaagaatagattttaaggctttgaaaatggttaatttctcaaaaacatcaatgtccaaacatctaccttttttcataggagtagacactagcaagctggacaaactatcacaaaagtatttgtcacacataacctgtggtctgttgctgattaatacagtactttttcttgtgtgattcttaacattatagcacaagtattatctcagtggattatccggaataacatctgaaagatgggttcatctatgtttgtgtttgctctttaaactattgtttctcctatcccaagttcgctttgcatctatcagtaaataaaattcttcagctgccttattaggagtgctatgagggtaacacctgttctgcttttcatcttgtatttagttgactgtattatttgatttcggattgaatgaatgtaaatagaaattaaatgcaaatttgaatgaacataaaaaaaaaaaaaaaa

SEQ ID NO: 11 - DNA, 4298 bp, Homo sapiens
cagacaggatattcactgctgtggcaaggcctgtagagagtttcgaagttaggaggactcaagacggtccctccctggacttttctgaaggggctcaaaagatgacacgcgccagagctggaaggcgtcgccaattggtccaacttttccctcctccctttttgcggatgagaaaaactgaggcccaggtttgggatttccagagcccgggatttcccggcaacgccgacaaccacattcccccggctattctgacccgccccggttccgggacgctccctgggagccgccgccgagggcctgctgggactcccggggaccccgccgtcggggcagcccccacgcccggcgccgcccgccggaacggcgccgctgttgcgcacttgcaggggagccggcgactgagggcgaggcagggagggagcaagcggggctgggagggctgctggcgcgggctcgccggctgtgtatggtctatcgcaggcagctgacctttgaggaggaaatcgctgctctccgctccttcctgtagtaacagccgccgctgccgccgccgccaggaaccccggccgggagcgagagccgcggggcgcagagccggcccggctgccggacggtgcggccccaccaggtgaacggccatggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggttgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctgggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcat

SEQ ID NO: 12 - DNA, 5156 bp, Homo sapiens
gaggtgagcgcggacgtcagagtggagagcggaaggtcagggaggctcggagcggaagtgagactagggagtctgtccgccattgtggacccgagaagcagagagcgagagggggaagaggagcgtgcaagcggaaaagacgggcctcttcctccgactcccgagcgcgaggccctcattttgggttctcagcgaacggcggcagcggcggcggctggaacaatcactcggccaagggcgacagccaactgctgtgagtgcacggggagaggcccaggcagcggcggcggcggcggctctcgggttgcggtgaagaatgtcagccactagcgtggatcagagacctaaagggcaaggaaataaagtttcagtacaaaacggttcgattcatcaaaaagatgctgtaaatgatgatgattttgagccatacttaagtagccagacaaatcagagtaacagctatccaccaatgtcagatccatacatgcctagttactatgctccatccattggatttccatattctcttggggaagcagcgtggtccacagctggagaccagcctatgccatatctgacaacctatggacaaatgagtaatggagaacatcactatataccagatggtgtatttagtcaacctggggcattaggaaatacccctccatttcttggtcaacatggatttaacttttttcctggtaatgctgatttctctacatgggggacaagtggatctcagggacaatcaacacaaagttctgcttatagtagcagttatggctatccacctagttctcttgggagagctattactgatggacaggctggatttggcaatgatactttgagtaaggtgcctggcattagcagtattgagcaaggcatgactggactgaaaattggtggtgacctgacagctgcagtgacaaaaactgtaggtacagctttgagcagcagtggtatgactagcattgcaaccaatagtgtgcccccagttagcagtgcagcacctaaaccaacctcctgggctgccattgccagaaagcctgccaaacctcaaccgaaacttaaacccaagggcaatgtgggaattgggggttctgctgtaccaccacctcctataaaacacaacatgaatattggaacttgggatgaaaaagggtcagtggtaaaggctccaccaacccaaccagttctgcctcctcaaactataatccagcagcctcagccattaattcaaccaccaccattggtgcaaagccaactgcctcaacagcagcctcaaccaccacaaccacagcagcaacaaggacctcagccacaggcccagcctcaccaagtgcagcctcaacagcagcagctgcagaatcgctgggtagctcctcgtaacaggggagcaggcttcaaccagaacaatggagcgggcagtgaaaactttggtttaggtgttgtacctgtcagtgcttcaccttctagtgtagaagtgcatcccgtgctggaaaagctaaaggccataaacaactataatcccaaagactttgattggaatctgaagaatggacgtgtgtttataattaaaagctactctgaggatgacatacatcgttccattaaatactctatctggtgtagtactgagcatggtaataagcgtttggatgcagcttaccgttccctgaatgggaaaggcccactctatttactcttcagtgtgaatggcagtggacatttttgtggagtggctgaaatgaagtctgttgtggactataatgcgtatgctggtgtctggtctcaggataagtggaagggcaaatttgaagttaaatggatctttgtcaaagatgttcccaataaccaattacggcatattcgcttagaaaataatgacaacaaaccggttaccaattcaagggacactcaagaggtacccctagaaaaagctaagcaagtgcttaaaataattgctactttcaagcataccacctcaatctttgatgactttgcacattatgaaaagcgtcaagaagaggaggaagccatgcgtagggagagaaatagaaacaaacaataaccgtatgaagatgtcctgttaaatttacaacactaacgatgtagactctggaaatgcctaataagtcaaagaagacgtattaaagctcttttctgcttaaggtgacatctttgaacactttaacacaaagttgactcttctcgtaatggttttcatcagcgcatctgcccttatactcttcaccaaacacacttgagaactgtaacttcgtcaagcactttctgtcctgaagcttttaccagtatctgctgtcttttgtaattatgcatcctagctaaggcacagaagactgaatgaatgcaaggattcattaactctttgaatttgttaaatactaacagttaaccattagaagtggttcaatgatgtaagagtcacactgcttcaactttttctttgttgtagtttttaaattgtcgatttttagctatttgacagattaaaagcaaaataatcatgccatatttagtcctggagttcaagtctaaatgttgatgtgaaaaattattgtagtaaacttttaatatggcaaagcaaccttaagctctattttagccaaatgaaacataatctgaaattatattagaacatttcccttgtcttcaaactgtttggtgtaacagaatattgatatgcagcttggtggatttcaccagttaatgcacattcttcttccctcctccccccattaatatgtatactgaaaaatgtgcatttgtctgaggaattattttgtttgctaccacttaatgaatctcaaaattttgagtaaatgtacctcagtctaatcagactttttatgacctttataactacatttaaaacccttaattcctatttctgggtgtttgcgagcctgattgctatcatgaagtaaaaatttattactctaggtattcactagctaaataaacatagttcttgtttagcaagcatatgttgttcctcagctcttttctccagcttttgcagtgtcctggcatccttaaaatactttgaaaatatggccttgatccatggattaaatcagtatctaagtgaatgtgttgatgttttattgatcagatctatataagtgggaatacagcatatatctggatattcttatagttatctttttaacatcttatttttttcattaattacatatcaacattaattttgtatcttgaagcaaattgattttgtataattaaatgtgtcaagcatctgtattaattgatttgatggcataaggttatgaaaataatgtactgccccatgtattactgttccaaaaggagaaagctatgtagaaagatacattaagggtgaaaatagcaatacagtagatttgaataccttgatgttttgcattacttcatttatgtttacatcatgtttagaaatgttttcatttactgtggtctttggtcacttcagctcaaagacctagtgatggatatttctttgaggctttcatttatataattttattttgtacaatgttttttttaaatgtgcaaatactgtattcaagtgaaaaaaatacagtatttgtagataaccatagctactacacagttcttcggtagtcccagtgtagttatatcagtgtttactgaagggaacatcaaaatattaatggtatattataaaataaagactttcttaaaggaaaattgcacctattttacctttttaagagtaagccatgaaatcttgtaacatgtctcttaactatttataatgaaaagtggcatttgggtatagtcaccacagcaatgttctacatccctaagattatctaggtaggacatgtcaaagatgactgttgtcattctggaggtcctattagagaatattataaaagggtgaccttgtaggaaggatctgagtcctccccctgaggttctctttttcttggtgctttattagcaactctggatatttttataaaactagttacattataaacggtttcaaacatgtttaatttacattaggtttttatgtaagagtgtcatggaagcactcagcaagcaggctgattgcaatagactcagacatgcgaataaatgtaattgagagtctattcatggtgaggagtacatcccagtgcctttaacctggatttctaatcttaagtgaaatgggtgcagcattcctttggaaaaaaaaatctttttattttcaagtgataattttgtgtttttctcatataagttttctccagagcacccaccttctcttccttcttggtctgtcattatattgcaaaatatttttcctctgaatgaaattatcacaggttgtctcaagcacaaccaactgaatgtctcttaactgtggggaccaaaagggagagagcctggggtctacaagaggagacacatcatcaaatgtttgaatgatcacaaattaagacattatcagcccagtaaatttcttgcttaatgtttttccaagttctggcttgaatatttcttattaaagctatcttatgtgggtattttattttgaaaggtattatagtttgtatatttaacagtaaggaggaaactgtaaccaaaattagtatttctctatacgtattggtacttgaagattcctttcaaaagaaatccagcgttttcctaattttagtacttaatttctctttttaatttaagtgatctttctaattcgaaagctgtgttctttttgaataccgtgcatgggggttaagctgatgttaaaacagtttgcaataaaaaaaaatgaatcagcttaagtcatttaatcatttcaagtgcattctgcatcctttaaaaataagtttaagaaatttaagagaattgtgttttcattaagttttgcatatcttttgttatgccatgtaaattccctttttcgtatgattaaaggaaggttatgataaaatgattagttcatttacattcacttgtagcaattacatgagaatttgaattttgtcgtgtttgggtttgttcattcctgtgaatgatggtacagttaggtgagattttctgttatggtacccaaactcaccatttggtcctctttaatctttgagggtttcaataaaaattgttcactcata

SEQ ID NO: 13 - DNA, 1218 bp, Homo sapiens
aaaggtctaggatgacatctggtgtattgactgtggccagtcttaaagctagtttttgctatgtggaacatgctgctctaattcagatttaaagagtttcttcctgttaattcgaagctcactgtgcctcttgtttccgagggaagaaggactgattaagtcatctaaatggatgcaatactgaattacaggtcagaagatactgaagattactacacattactgggatgtgatgaactatcttcggttgaacaaatcctggcagaatttaaagtcagagctctggaatgtcacccagacaagcatcctgaaaaccccaaagctgtggagacttttcagaaactgcagaaggcaaaggagattctgaccaatgaagagagtcgagcccgctatgaccactggcgaaggagccagatgtcgatgccattccagcagtgggaagctttgaatgactcagtgaagacgtcaatgcactgggttgtcagaggtaaaaaagacctgatgctggaagaatctgacaagactcataccaccaagatggaaaatgaggaatgtaatgagcaaagagaaagaaagaaagaggagctggcttcaaccgcagagaaaacggagcagaaagaacccaagcccctagagaagtcagtctccccgcaaaattcagattcttcaggttttgcagatgtgaatggttggcaccttcgtttccgctggtccaaggatgctccctcagaactcctgaggaagttcagaaactatgaaatatgaaatatctctgcttcaaaaaatgaggaagagcaagactgtcccctatgctgccaacatgcagtctttgtttatgtcttaaaaatgtcatgtttatgtcatgtctgtgaattgctgagtactaattgattcctccatccttgaatcagttctcataatgctttttaaataagaaaaattcagaagatgaatttcttccaatatttgaataaattaaagctcttagatacagagtagattgtattatatgctttttcctattaatactacttatagaaatccattaaaaagcaatctctgtacagtgtatttaaatatttcattgacatactgtgatctctattagtgatggatgtacaaaaaatgttttcttacccttgacttacaatgaaatgtgaaattacttgtctgaaccccgtggggagaaataaataattttcccaaagttcaaaaaaa

SEQ ID NO: 14 - DNA, 5889 bp, Homo sapiens
gctgccagctgagtttttttgctgctttgagtctcagttttctttctttcctagagtctctgaagccacagatctcttaagaactttctgtctccaaaccgtggctgctcgataaatcagacagaacagttaatcctcaatttaagcctgatctaacccctagaaacagatatagaacaatggaagtgacaacaagattgacatggaatgatgaaaatcatctgcgcaagctgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacatgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgatatcaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaactatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgcaaaaaaaaaaaaaaaaaataagaaagaacaaaacaaaccccacaaaaattagctgggtatgatggcacgtgcctgtagtcccagttactcaggatgattgattgagccttggaggtggaggctacagtgagctgagattgtgccactgtactctagccagggagaaagagtgagatcctggctcaaaaaaaccaaataaaacaaaacaaacaaacgaaaaacagaaaggaagactgaaagagaatgaaaagctggggagaggaaataaaaataaagaaggaagagtgtttcatttatatctgaatgaaaatatgaatgactctaagtaattgaattaattaaaatgagccaacttttttttaacaatttacattttatttctatgggaaaaaataaatattcctcttctaacaaacccatgcttgattttcattaattgaattccaaatcatcctagccatgtgtccttccatttaggttactggggcaaatcagtaagaaagttcttatatttatgctccaaataattctgaagtcctcttactagctgtgaaagctagtactattaagaaagaaaacaaaattcccaaaagatagctttcacttttttttttccttaaagacttcctaattctcttctccaaattcttagtcttcttcaaaataatatgctttggttcaatagttatccacattctgacagtctaatttagttttaatcagaattatactcatcttttgggtagtcatagatattaagaaagcaagagtttcttatgtccagttatggaatatttcctaaagcaaggctgcaggtgaagttgtgctcaagtgaatgttcaggagacacaattcagtggaagaaattaagtctttaaaaaagacctaggaataggagaaccatggaaattgaggaggtaggcctacaagtagatattgggaacaaaattagagaggcaaccagaaaaagttattttaggctcaccagagttgttcttattgcacagtaacacaccaatataccaaaacagcaggtattgcagtagagaaagagtttaataattgaatggcagaaaaatgaggaaggttgaggaaacctcaaatctacctccctgctgagtctaagtttaggatttttaagagaaaggcaggtaaggtgctgaaggtctggagctgctgatttgttggggtatagggaatgaaatgaaacatacagagatgaaaactggaagtttttttttgtttgttttgttttttttttgttgttgtttttttttttttttgtttttttgctgagtcaattccttggagggggtcttcagactgactggtgtcagcagacccatgggattccaagatctggaaaactttttagatagaaacttgatgtttcttaacgttacatatattatcttatagaaataactaagggaagttagtgccttgtgaccacatctatgtgacttttaggcagtaagaaactataaggaaaggagctaacagtcatgctgtaagtagctacagggaattggcttaaagggcaagttggttagtacttagctgtgtttttattcaaagtctacattttatgtagtggttaatgtttgctgttcattaggatggtttcacagttaccatacaaatgtagaagcaacaggtccaaaaagtagggcatgattttctccatgtaatccagggagaaaacaagccatgaccattgttggttgggagactgaaggtgattgaaggttcaccatcatcctcaccaacttttgggccataattcacccaaccctttggtggagcctgaaaaaaatctgggcagaatgtaggacttctttattttgtttaaaggggtaacacagagtgcccttatgaaggagttggagatcctgcaaggaagagaaggagtgaaggagagatcaagagagagaaacaatgaggaacatttcatttgacccaacatcctttaggagcataaatgttgacactaagttatcccttttgtgctaaaatggacagtattggcaaaatgataccacaacttcttattctctggctctatattgctttggaaacacttaaacatcaaatggagttaaatacatatttgaaatttaggttaggaaatattggtgaggaggcctcaaaaagggggaaacatcttttgtctgggaggatattttccattttgtggatttccctgatctttttctaccaccctgaggggtggtgggaattatcattttgctacattttagaggtcatccaggatttttgaaactttacattctttacggttaagcaagatgtacagctcagtcaaagacactaaattcttcttagaaaaatagtgctaaggagtatagcagatgacctatatgtgtgttggctgggagaatatcatcttaaagtgagagtgatgttgtggagacagttgaaatgtcaatgctagagcctctgtggtgtgaatgggcacgttaggttgttgcattagaaagtgactgtttctgacagaaatttgtagctttgtgcaaactcacccaccatctacctcaataaaatatagagaaaagaaaaatagagcagtttgagttctatgaggtatgcaggcccagagagacataagtatgttcctttagtcttgcttcctgtgtgccacactgcccctccacaaccatagctgggggcaattgtttaaagtcattttgttcccgactagctgccttgcacattatcttcattttcctggaatttgatacagagagcaatttatagccaattgatagcttatgctgtttcaatgtaaattcgtggtaaataacttaggaactgcctcttctttttctttgaaaacctacttataactgttgctaataagaatgtgtattgttcaggacaacttgtctccatacagttgggttgtaaccctcatgcttggcccaaataaactctctacttatatcagtttttcctacacttcttccttttaggtcaacaataccaagaggggttactgtgctgggtaatgtgtaaacttgtgtcttgtttagaaagataaatttaaagactatcacattgctttttcataaaacaagacaggtctacaattaatttattttgacgcaaattgataggggggccaagtaagccccatatgcttaatgatcagctgatgaataatcatctcctagcaacataactcaatctaatgctaaggtacccacaagatggcaaggctgatcaaagtcgtcatggaatcctgcaaccaaaagccatgggaatttggaagccctcaaatcccattcctaatctgatgagtctatggaccaatttgtggaggacagtagattaaatagatctgatttttgccatcaatgtaaggaggataaaaacttgcataccaattgtacacccttgcaaaatctttctctgatgttggagaaaatgggccagtgagatcatggatatagaagtacagtcaatgttcagctgtaccctcccacaatcccacttccttcctcaacacaattcaaacaaatagactcagactgtttcaggctccaggacaggaagtgcagtgtaggcaaaattgcaaaaattgagggcacaggggtggaggtgggggggttgaataacaagctgtgctaaataattacgtgtaaatatattttttcatttttaaaaattgatttcttttgcacattccatgacaatatatgtcacatttttaaaataaatgcaaagaagcatacatccaaaaaaaaaaaaaaaaa

SEQ ID NO: 15 - DNA, 1942 bp, Homo sapiens
gcagtgcggcggtcacaggctgagtgctgcggcgcgatccttgcttccctgagcgttggcccgggaggaaagaagatggtgctggatctggatttgtttcgggtggataaaggaggggacccagccctcatccgagagacgcaggagaagcgcttcaaggacccgggactagtggaccagctggtgaaggcagacagcgagtggcgacgatgtagatttcgggcagacaacttgaacaagctgaagaacctatgcagcaagacaatcggagagaaaatgaagaaaaaagagccagtgggagatgatgagtctgtcccagagaatgtgctgagtttcgatgaccttactgcagacgctttagctaacctgaaagtctcacaaatcaaaaaagtccgactcctcattgatgaagccatcctgaagtgtgacgcggagcggataaagttggaagcagagcggtttgagaacctccgagagattgggaaccttctgcacccttctgtacccatcagtaacgatgaggatgtggacaacaaagtagagaggatttggggtgattgtacagtcaggaagaagtactctcatgtggacctggtggtgatggtagatggctttgaaggcgaaaagggggccgtggtggctgggagtcgagggtacttcttgaagggggtcctggtgttcctggaacaggctctcatccagtatgcccttcgcaccttgggaagtcggggctacattcccatttataccccctttttcatgaggaaggaggtcatgcaggaggtggcacagctcagccagtttgatgaagaactttataaggtgattggcaaaggcagtgaaaagtctgatgacaactcctatgatgagaagtacctgattgccacctcagagcagcccattgctgccctgcaccgggatgagtggctccggccggaggacctgcccatcaagtatgctggcctgtctacctgcttccgtcaggaggtgggctcccatggccgtgacacccgtggcatcttccgagtccatcagtttgagaagattgaacagtttgtgtactcatcaccccatgacaacaagtcatgggagatgtttgaagagatgattaccaccgcagaggagttctaccagtccctggggattccttaccacattgtgaatattgtctcaggttctttgaatcatgctgccagtaagaagcttgacctggaggcctggtttccgggctcaggagccttccgtgagttggtctcctgttctaattgcacggattaccaggctcgccggcttcgaatccgatatgggcaaaccaagaagatgatggacaaggtggagtttgtccatatgctcaatgctaccatgtgcgccactacccgtaccatctgcgccatcctggagaactaccagacagagaagggcatcactgtgcctgagaaattgaaggagttcatgccgccaggactgcaagaactgatcccctttgtgaagcctgcgcccattgagcaggagccatcaaagaagcagaagaagcaacatgagggcagcaaaaagaaagcagcagcaagagacgtcaccctagaaaacaggctgcagaacatggaggtcaccgatgcttgaacattcctgcctccctatttgccaggctttcatttctgtctgctgagatctcagagcctgcccaacagcagggaagccaagcacccattcatccccctgcccccatctgactgcgtagctgagaggggaacagtgccatgtaccacacagatgttcctgtctcctcgcatgggcatagggacccatcattgatgactgatgaaaccatgtaataaagcatctctggggagggcttaggactcttcctcagtcttcttccccgggcttgaaccccgaaaaaaaaaaaaaaaaaaaaaaaaaaaaaaaaaaaaaaaaaaaaaaa

SEQ ID NO: 16 - DNA, 3866 bp, Homo sapiens
gagttctgctccggcgcccccgagcaccgcccgcttcagccgaccagccccgtcggctactgggcctcgccgagacgagaggagggaaaggcctcggcggccgcgaggaggcggcgggggcgcgggcggaggcggcgcgggcggccgcggctgccgctttgttgtgcggcccgggccgaggaaggagaagtgggaggaggggggagctcggcgtcccgctccctccgcggctcatggcgacgactctcggcacatccgggaccctccggccgtggcggccgaggcgccggctgctcgggccccagccccggccgctgtggtgactccgccgcgcctcgccgtcgcccccgtgccgcccgccgcccccgccgcccccgccgccggggacatgtctaaccccggaggccggaggaacgggcccgtcaagctgcgcctgacagtactctgtgcaaaaaacctggtgaaaaaggattttttccgacttcctgatccatttgctaaggtggtggttgatggatctgggcaatgccattctacagatactgtgaagaatacgcttgatccaaagtggaatcagcattatgacctgtatattggaaagtctgattcagttacgatcagtgtatggaatcacaagaagatccataagaaacaaggtgctggatttctcggttgtgttcgtcttctttccaatgccatcaaccgcctcaaagacactggttatcagaggttggatttatgcaaactcgggccaaatgacaatgatacagttagaggacagatagtagtaagtcttcagtccagagaccgaataggcacaggaggacaagttgtggactgcagtcgtttatttgataacgatttaccagacggctgggaagaaaggagaaccgcctctggaagaatccagtatctaaaccatataacaagaactacgcaatgggagcgcccaacacgaccggcatccgaatattctagccctggcagacctcttagctgctttgttgatgagaacactccaattagtggaacaaatggtgcaacatgtggacagtcttcagatcccaggctggcagagaggagagtcaggtcacaacgacatagaaattacatgagcagaacacatttacatactcctccagacctaccagaaggctatgaacagaggacaacgcaacaaggccaggtgtatttcttacatacacagactggtgtgagcacatggcatgatccaagagtgcccagggatcttagcaacatcaattgtgaagagcttggtccattgcctcctggatgggagatccgtaatacggcaacaggcagagtttatttcgttgaccataacaacagaacaacacaatttacagatcctcggctgtctgctaacttgcatttagttttaaatcggcagaaccaattgaaagaccaacagcaacagcaagtggtatcgttatgtcctgatgacacagaatgcctgacagtcccaaggtacaagcgagacctggttcagaaactaaaaattttgcggcaagaactttcccaacaacagcctcaggcaggtcattgccgcattgaggtttccagggaagagatttttgaggaatcatatcgacaggtcatgaaaatgagaccaaaagatctctggaagcgattaatgataaaatttcgtggagaagaaggccttgactatggaggcgttgccagggaatggttgtatctcttgtcacatgaaatgttgaatccatactatggcctcttccagtattcaagagatgatatttatacattgcagatcaatcctgattctgcagttaatccggaacatttatcctatttccactttgttggacgaataatgggaatggctgtgtttcatggacattatattgatggtggtttcacattgcctttttataagcaattgcttgggaagtcaattaccttggatgacatggagttagtagatccggatcttcacaacagtttagtgtggatacttgagaatgatattacaggtgttttggaccataccttctgtgttgaacataatgcatatggtgaaattattcagcatgaacttaaaccaaatggcaaaagtatccctgttaatgaagaaaataaaaaagaatatgtcaggctctatgtgaactggagatttttacgaggcattgaggctcaattcttggctctgcagaaaggatttaatgaagtaattccacaacatctgctgaagacatttgatgagaaggagttagagctcattatttgtggacttggaaagatagatgttaatgactggaaggtaaacacccggttaaaacactgtacaccagacagcaacattgtcaaatggttctggaaagctgtggagttttttgatgaagagcgacgagcaagattgcttcagtttgtgacaggatcctctcgagtgcctctgcagggcttcaaagcattgcaaggtgctgcaggcccgagactctttaccatacaccagattgatgcctgcactaacaacctgccgaaagcccacacttgcttcaatcgaatagacattccaccctatgaaagctatgaaaagctatatgaaaagctgctaacagccattgaagaaacatgtggatttgctgtggaatgacaagcttcaaggatttacccaggactctatttatacaaccctgactgacagcctcctttcagcagagtttcaaagaatatgctgaaatacaggaaaacactccccccccccccctttttttttttttttttacattttaggacactgtgaggggaaaggacaattttgaaattccttttcaaggaaaaaaaaggtctttatgctttgccatgaggccacattcagctgctatttaaacttaatatcttgaacctaaagaatgctgacttttcctacatttccagagttaggcagtattctacacttaaagactactactatttttataaaaggtaatctattcaaatttcttcacagatttcaagtctctcaaacatcaagacaacttcagcagtcggtacaagtcacatttcattttgattgaatacatgatcttgaacagctcctgtacttgctctttgtaaaaaaaaataaaattattttgaattattctacctttgtaaacaattggctaaaagaatcatctttaagaaattaagccatttacatgtttgtgtttttctatagcagagcattatattttgcattatatgtttcaacctagtctaagtgggtcttttttacatttttcaagaacggatttcctggaatacagcgatataattttggttgtcaaattcctaatgcaaccatttagtctaaacttagtcatttatttgtgacaataagatgtgttcaggggctccctgtttttaagagactcttttaaaaaaaaaaaaacctaatgtttttatcttgagtcaatatgattaggtattttggatttacttttaatcttaaaatactgcatttttatagcttctcagagcatgtggatgggatgggattttcgttattttgctgggtcagcttatctttaatatatggactattcctataaaccaaagtctctgacaagtgcacctaatttatattgtattttaactacagtgtaagtttccattaacaaaaccatcctaaagcgttaactgctcataattttaatcagctacagttatgaaaaaggaagaattttgctctaaagattattaataagcttagaaaatcctgcttttacctaacagaatgaagtggggttgaggggcggtagtatgaacagtgattctaactctatggcgtaaattactttgaagagttcattttggaagtaagggaatcccgcttcccttgg

SEQ ID NO: 17 - DNA, 3222 bp, Homo sapiens
ccgggtgagacaaatcgggcgccatcttgtcttgttcccgaagaagtagaagcatcgaaagcgttggagaggtgttaccggaacggcggcgacaagggtgttcccgaactagagtggggcatacataatcttgctgctatgcttcgaagctgtagtctgaatcaacctaagttttaaacagaaggtgaacctctgagatagaaaatcaagtatattttaaaagaagggatgtgggatcaaggaggacagccttggcagcagtggcccttgaaccagcaacaatggatgcagtcattccagcaccaacaggatccaagccagattgattgggctgcattggcccaagcttggattgcccaaagagaagcttcaggacagcaaagcatggtagaacaaccaccaggaatgatgccaaatggacaagatatgtctacaatggaatctggtccaaacaatcatgggaatttccaaggggattcaaacttcaacagaatgtggcaaccagaatggggaatgcatcagcaacccccacacccccctccagatcagccatggatgccaccaacaccaggcccaatggacattgttcctccttctgaagacagcaacagtcaggacagtggggaatttgcccctgacaacaggcatatatttaaccagaacaatcacaactttggtggaccacccgataattttgcagtggggccagtgaaccagtttgactatcagcatggggctgcttttggtccaccgcaaggtggatttcatcctccttattggcaaccaggacctccaggacctccagcacctccccagaatcgaagagaaaggccatcatcattcagggatcgtcagcgttcacctattgcacttcctgtgaagcaggagcctccacaaattgacgcagtaaaacgcaggactcttcccgcttggattcgcgaaggtcttgaaaaaatggaacgtgaaaagcagaagaaattggagaaagaaagaatggaacaacaacgttcacaattgtccaaaaaagaaaaaaaggccacagaagatgctgaaggaggggatggccctcgtttacctcagagaagtaaatttgatagtgatgaggaagaagaagacactgaaaatgttgaggctgcaagtagtgggaaagtcaccagaagtccatccccagttcctcaagaagagcacagtgaccctgagatgactgaagaggagaaagagtatcaaatgatgttgctgacaaaaatgcttctaacagaaattctgctggatgtcacagatgaagaaatttattacgtagccaaagatgcacaccgcaaagcaacgaaagctcctgcaaaacagctggcacagtccagtgcactggcttccctcactggactcggtggactgggtggttatggatcaggagacagtgaagatgagaggagtgacagaggatctgagtcatctgacactgatgatgaagaattacggcatcgaatccggcaaaaacaggaagctttttggagaaaagaaaaagaacagcagctattacatgataaacagatggaagaagaaaagcagcaaacagaaagggttacaaaagagatgaatgaatttatccataaagagcaaaatagtttatcactactagaagcaagagaagcagacggtgatgtggttaatgaaaagaagagaactccaaatgaaaccacatcagttttagaaccaaaaaaagagcataaagaaaaagaaaaacaaggaaggagtaggtcgggaagttctagtagtggtagttccagtagcaatagcagaactagtagtactagtagtactgtctctagctcttcatacagttctagctcaggtagtagtcgtacttcttctcggtcttcttctcctaaaaggaaaaagagacacagtaggagtagatctccaacaatcaaagctagacgtagcaggagtagaagctattctcgcagaattaaaatagagagcaatagggctagggtaaagattagagatagaaggagatctaatagaaatagcattgaaagagaaagacgacgaaatcggagtccttcccgagagagacgtagaagtagaagtcgctcaagggatagacgaaccaatcgtgccagtcgcagtaggagtcgagataggcgtaaaattgatgatcaacgtggaaatcttagtgggaacagtcataagcataaaggtgaggctaaagaacaagagaggaaaaaggagaggagtcgaagtatagataaagataggaaaaagaaagacaaagaaagggaacgtgaacaggataaaagaaaagagaaacaaaaaagggaagaaaaagattttaagttcagtagtcaggatgatagattaaaaaggaaacgagaaagtgaaagaacattttctaggagtggttctatatctgttaaaatcataagacatgattctagacaggatagtaagaaaagtactaccaaagatagtaaaaaacattcaggctctgattctagtggaaggagcagttctgagtctccaggaagtagcaaagaaaagaaggctaagaagcctaaacatagtcgatcgcgatccgtggagaaatctcaaaggtctggtaagaaggcaagccgcaaacacaagtctaagtcccgatcaaggtagtatactttttaaagtattttgtctgatttttaaaaaaaattgactgaatttattcaaagttgaaagtgtcctttctctctctctttaataaactcagtttggtacttgataaataatcatagtcttaaatgttagaaatcctatataatattatttatttaaaattgcagatttttaatttaaaatacatttttatttttaaattttgtcttttcccttttttttcagatcaacaacccctccccgtcgtaaacgctgaggaatgatgtggcaagaatgccatgatgttctttaaaaaaattccatgagttttaagggcttgtctcattatagaggcacattgtggctgtgtaggtgaaaccagaatcttttttttttttaatctgtaaataggtgtactttttccaatgctgctccaagttacttaataggatttctttgtattacgtttttttcaaaaaatatagtgcataataagactataaacatgccattctctttcagctgtaatgttcttaaaattattcttgaatgtactgtgatgtcaataaagctctttagttcatttttgttaaa

SEQ ID NO: 18 - DNA, 3546 bp, Homo sapiens
gctgacgcgcgctgacgcgcggagactttaggtgcatgttggcagcggcagcgcaagccacataaacaaaggcacattggcggccagggccagtccgcccggcggctcgcgcacggctccgcggtcccttttgcctgtccagccggccgcctgtccctgctccctccctccgtgaggtgtccgggttcccttcgcccagctctcccacccctacccgaccccggcgcccgggctcccagagggaactgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataatgtataatgggaaatgtttggccatgcttggttgttgcagttcagacaaatgtaacacacacacacatacacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaacagaaaaaaaaaaaaaaaaa

SEQ ID NO: 19 - DNA, 2334 bp, Homo sapiens
cttgctcacggctctgcgactccgacgccggcaaggtttggagagcggctgggttcgcgggacccgcgggcttgcacccgcccagactcggacgggctttgccaccctctccgcttgcctggtcccctctcctctccgccctcccgctcgccagtccatttgatcagcggagactcggcggccgggccggggcttccccgcagcccctgcgcgctcctagagctcgggccgtggctcgtcggggtctgtgtcttttggctccgagggcagtcgctgggcttccgagaggggttcgggccgcgtaggggcgctttgttttgttcggttttgtttttttgagagtgcgagagaggcggtcgtgcagacccgggagaaagatgtcaaacgtgcgagtgtctaacgggagccctagcctggagcggatggacgccaggcaggcggagcaccccaagccctcggcctgcaggaacctcttcggcccggtggaccacgaagagttaacccgggacttggagaagcactgcagagacatggaagaggcgagccagcgcaagtggaatttcgattttcagaatcacaaacccctagagggcaagtacgagtggcaagaggtggagaagggcagcttgcccgagttctactacagacccccgcggccccccaaaggtgcctgcaaggtgccggcgcaggagagccaggatggcagcgggagccgcccggcggcgcctttaattggggctccggctaactctgaggacacgcatttggtggacccaaagactgatccgtcggacagccagacggggttagcggagcaatgcgcaggaataaggaagcgacctgcaaccgacgattcttctactcaaaacaaaagagccaacagaacagaagaaaatgtttcagacggttccccaaatgccggttctgtggagcagacgcccaagaagcctggcctcagaagacgtcaaacgtaaacagctcgaattaagaatatgtttccttgtttatcagatacatcactgcttgatgaagcaaggaagatatacatgaaaattttaaaaatacatatcgctgacttcatggaatggacatcctgtataagcactgaaaaacaacaacacaataacactaaaattttaggcactcttaaatgatctgcctctaaaagcgttggatgtagcattatgcaattaggtttttccttatttgcttcattgtactacctgtgtatatagtttttaccttttatgtagcacataaactttggggaagggagggcagggtggggctgaggaactgacgtggagcggggtatgaagagcttgctttgatttacagcaagtagataaatatttgacttgcatgaagagaagcaattttggggaagggtttgaattgttttctttaaagatgtaatgtccctttcagagacagctgatacttcatttaaaaaaatcacaaaaatttgaacactggctaaagataattgctatttatttttacaagaagtttattctcatttgggagatctggtgatctcccaagctatctaaagtttgttagatagctgcatgtggcttttttaaaaaagcaacagaaacctatcctcactgccctccccagtctctcttaaagttggaatttaccagttaattactcagcagaatggtgatcactccaggtagtttggggcaaaaatccgaggtgcttgggagttttgaatgttaagaattgaccatctgcttttattaaatttgttgacaaaattttctcattttcttttcacttcgggctgtgtaaacacagtcaaaataattctaaatccctcgatatttttaaagatctgtaagtaacttcacattaaaaaatgaaatattttttaatttaaagcttactctgtccatttatccacaggaaagtgttatttttaaaggaaggttcatgtagagaaaagcacacttgtaggataagtgaaatggatactacatctttaaacagtatttcattgcctgtgtatggaaaaaccatttgaagtgtacctgtgtacataactctgtaaaaacactgaaaaattatactaacttatttatgttaaaagattttttttaatctagacaatatacaagccaaagtggcatgttttgtgcatttgtaaatgctgtgttgggtagaataggttttcccctcttttgttaaataatatggctatgcttaaaaggttgcatactgagccaagtataattttttgtaatgtgtgaaaaagatgccaattattgttacacattaagtaatcaataaagaaaacttccatagctaaaaaaaaaaaaaaaaaaa

SEQ ID NO: 20 - DNA, 1949 bp, Homo sapiens
gggagagcagtttacgacagcgccggtcgtgtttacggcggcgcccgctgcgcgcgcatgtttcctcttttcctggtttctcaagagtgctgctgctaacgcggtccccggcacgcaccatctgttgccatcccggccggccgaggccattgcagattttggaagatggcaaagttcatgacacccgtgatccaggacaacccctcaggctggggtccctgtgcggttcccgagcagtttcgggatatgccctaccagccgttcagcaaaggagatcggctaggaaaggttgcagactggacaggagccacataccaagataagaggtacacaaataagtactcctctcagtttggtggtggaagtcaatatgcttatttccatgaggaggatgaaagtagcttccagctggtggatacagcgcgcacacagaagacggcctaccagcggaatcgaatgagatttgcccagaggaacctccgcagagacaaagatcgtcggaacatgttgcagttcaacctgcagatcctgcctaagagtgccaaacagaaagagagagaacgcattcgactgcagaaaaagttccagaaacaatttggggttaggcagaaatgggatcagaaatcacagaaaccccgagactcttcagttgaagttcgtagtgattgggaagtgaaagaggaaatggattttcctcagttgatgaagatgcgctacttggaagtatcagagccacaggacattgagtgttgtggggccctagaatactacgacaaagcctttgaccgcatcaccacgaggagtgagaagccactgcggagcatcaagcgcatcttccacactgtcaccaccacagacgaccctgtcatccgcaagctggcaaaaactcaggggaatgtgtttgccactgatgccatcctggccacgctgatgagctgtacccgctcagtgtattcctgggatattgtcgtccagagagttgggtccaaactcttctttgacaagagagacaactctgactttgacctcctgacagtgagtgagactgccaatgagccccctcaagatgaaggtaattccttcaattcaccccgcaacctggccatggaggcaacctacatcaaccacaatttctcccagcagtgcttgagaatggggaaggaaagatacaacttccccaacccaaacccgtttgtggaggacgacatggataagaatgaaatcgcctctgttgcgtaccgttaccgcaggtggaagcttggagatgatattgaccttattgtccgttgtgagcacgatggcgtcatgactggagccaacggggaagtgtccttcatcaacatcaagacactcaatgagtgggattccaggcactgtaatggcgttgactggcgtcagaagctggactctcagcgaggggctgtcattgccacggagctgaagaacaacagctacaagttggcccggtggacctgctgtgctttgctggctggatctgagtacctcaagcttggttatgtgtctcggtaccacgtgaaagactcctcacgccacgtcatcctaggcacccagcagttcaagcctaatgagtttgccagccagatcaacctgagcgtggagaatgcctggggcattttacgctgcgtcattgacatctgcatgaagctggaggagggcaaatacctcatcctcaaggaccccaacaagcaggtcatccgtgtctacagcctccctgatggcaccttcagctctgatgaagatgaggaggaagaggaggaggaagaagaggaagaagaagaggaagaaacttaaaccagtgatgtggagctggagtttgtccttccaccgagactacgagggcctttgatgcttagtggaatgtgtgtctaacttgctctctgacatttagcagatgaaataaaatatatatctgtttagtctttccctca

SEQ ID NO: 21 - DNA, 1869 bp, Homo sapiens
tacctggttgatcctgccagtagcatatgcttgtctcaaagattaagccatgcatgtctaagtacgcacggccggtacagtgaaactgcgaatggctcattaaatcagttatggttcctttggtcgctcgctcctctcccacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 22 - DNA, 753 bp, Homo sapiens
ctctttctcagtgaccgggtggtttgcttaggcgcagacggggaagcggagccaacatgccagtggcccggagctgggtttgtcgcaaaacttatgtgaccccgcggagacccttcgagaaatctcgtctcgaccaagagctgaagctgatcggcgagtatgggctccggaacaaacgtgaggtctggagggtcaaatttaccctggccaagatccgcaaggccgcccgggaactgctgacgcttgatgagaaggacccacggcgtctgttcgaaggcaacgccctgctgcggcggctggtccgcattggggtgctggatgagggcaagatgaagctggattacatcctgggcctgaagatagaggatttcttagagagacgcctgcagacccaggtcttcaagctgggcttggccaagtccatccaccacgctcgcgtgctgatccgccagcgccatatcagggtccgcaagcaggtggtgaacatcccgtccttcattgtccgcctggattcccagaagcacatcgacttctctctgcgctctccctacgggggtggccgcccgggccgcgtgaagaggaagaatgccaagaagggccagggtggggctggggctggagacgacgaggaggaggattaagtccacctgtccctcctgggctgctggattgtctcgttttcctgccaaataaacaggatcagcgctttacaaaaaaaaaaaaaaaaaaaaaaaaaaaaaaaaaaaaaaaaaa

SEQ ID NO: 23 - DNA, 1804 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctta

SEQ ID NO: 24 - DNA, 2567 bp, Homo sapiens
ctccggcgcagtgttgggactgtctgggtatcggaaagcaagcctacgttgctcactattacgtataatccttttcttttcaagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcacaaagcacaacgatgatgaacagtatgcttgggagtcttctgctggaggttccttcactgtgcgtgctgaccatggtgagcccattggcaggggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaaacccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgttttaaaaaaaaaaaaaaaaaaaaaaaaaaaaa

SEQ ID NO: 25 - DNA, 25 bp, Homo sapiens
gaatgcccagagggctgcagctatg

SEQ ID NO: 26 - DNA, 25 bp, Homo sapiens
gacactggcggtggactgcgtaggg

SEQ ID NO: 27 - DNA, 25 bp, Homo sapiens
gcagcacaacagaggcgggtatttc

SEQ ID NO: 28 - DNA, 25 bp, Homo sapiens
tttttacctcagggacctgttgcaa

SEQ ID NO: 29 - DNA, 25 bp, Homo sapiens
agaggaggccagggctcgacccaca

SEQ ID NO: 30 - DNA, 25 bp, Homo sapiens
tgcctggagagagccaaagagttca

SEQ ID NO: 31 - DNA, 25 bp, Homo sapiens
aaaagagagcctccttttagaggac

SEQ ID NO: 32 - DNA, 25 bp, Homo sapiens
aatgaacctggaagctcgaaaaaca

SEQ ID NO: 33 - DNA, 25 bp, Homo sapiens
ggatgaacgacagcttggtgatcca

SEQ ID NO: 34 - DNA, 25 bp, Homo sapiens
aagacactgcctgcttttacttggc

SEQ ID NO: 35 - DNA, 25 bp, Homo sapiens
actcctgtgctggctaaagctgttg

SEQ ID NO: 36 - DNA, 25 bp, Homo sapiens
ggaagccatgcgtagggagagaaat

SEQ ID NO: 37 - DNA, 25 bp, Homo sapiens
cagtgaagacgtcaatgcactgggt

SEQ ID NO: 38 - DNA, 25 bp, Homo sapiens
cataaccgagcggtataggatatat

SEQ ID NO: 39 - DNA, 25 bp, Homo sapiens
gcgacgatgtagatttcgggcagac

SEQ ID NO: 40 - DNA, 25 bp, Homo sapiens
ggagcgcccaacacgaccggcatcc

SEQ ID NO: 41 - DNA, 25 bp, Homo sapiens
caggatccaagccagattgattggg

SEQ ID NO: 42 - DNA, 25 bp, Homo sapiens
tggactattccaggttccaggcgaa

SEQ ID NO: 43 - DNA, 25 bp, Homo sapiens
tgcaaccgacgattcttctactcaa

SEQ ID NO: 44 - DNA, 25 bp, Homo sapiens
gagtgggattccaggcactgtaatg

SEQ ID NO: 45 - DNA, 25 bp, Homo sapiens
tggagggcaagtctggtgccagcag

SEQ ID NO: 46 - DNA, 25 bp, Homo sapiens
gcgccatatcagggtccgcaagcag

SEQ ID NO: 47 - DNA, 25 bp, Homo sapiens
cctttgccgatccgccgcccgtcca

SEQ ID NO: 48 - DNA, 25 bp, Homo sapiens
gcatgatcaagctaggtctaggtat